SEQUENCE LISTING (39 sequences)

SEQ ID NO: 1 - DNA, 1023 bp, Artificial Sequence
  Synthetic polynucleotide
cgtaagcggtgctatagtacaagtgcggtcgtactggcggctgttaccttgttcgcgctcagtgtggatcgtggtgtaatcgcggatagcttttcggcgaaccaggagattcggtattccgaagtcacaccatatcatgtaacgtctgtatggacgaaaggtgtaacgcctccggccaaattcacacagggcgaggacgtattccacgccccatatgtcgccaaccaaggttggtatgatattacgaagacgtttaacggcaaggacgacctgttgtgcggggcggccaccgcagggaacatgttgcattggtggtttgaccaaaacaaagaaaaaatcgaagcctatctgaaaaagcacccagataagcagaaaattatgttcggcgatcaagagctcctcgatgtccggaaagtgattaacacgaagggggatcagacgaatagtgagctgtttaactattttcgcgacaaagctttcccggggttatccgcccgtcgcattggcgtcatgcctgacctcgtacttgacatgttcattaatgggtattatctgaatgtatacaaaacccagacaactgatgtaaatcgcacatatcaggagaaagatcggcggggtggcatttttgatgccgtatttacacgtggtgaccagtccaaattattaactagccgtcatgatttcaaggagaagaacttaaaggaaatctctgatttaatcaaaaaggaattgactgaaggtaaggctttaggccttagccatacttatgcaaatgtgcgtattaaccatgttattaacttgtggggggcagactttgactcgaacggcaatttaaaagccatctatgtcacggacagcgacagcaatgcttcgattggtatgaaaaagtattttgtaggcgtaaattcagcgggtaaagttgctatcagtgccaaagagatcaaggaggacaatatcggcgctcaggttcttggtttattcacgctgagcacaggccaagattcttggaatcaaacaaactaa

SEQ ID NO: 2 - DNA, 1023 bp, Artificial Sequence
  Synthetic polynucleotide
cgtaagcgctgttactcgaccagtgcggttgtactggctgcggtgaccctttttgccctctcggtggaccgtggggtcattgcagattctttctccgcgaatcaagagattcggtattctgaggtgactccttaccacgtaacatcggtatggactaaaggcgttacgccgcctgctaaattcactcagggtgaggatgtattccatgccccttatgtggctaaccagggttggtacgacatcacgaaaaccttcaatggcaaagacgacctcttatgtggcgcagcgactgcggggaacatgttgcactggtggttcgatcaaaataaagaaaaaattgaggcatatctgaaaaagcaccctgacaagcaaaagattatgttcggcgaccaggaactgcttgacgtgcgtaaggtgatcaacactaaaggggaccaaaccaactccgaactgtttaattatttccgcgacaaagctttccctggtttatcggcacggcggattggcgttatgccggatctggtgttggacatgtttattaatggctattaccttaacgtatacaaaacacaaactacagatgtaaatcgcacatatcaagagaaggaccgtcgtgggggcattttcgatgccgtattcacacgtggggatcaaagtaagcttctgacctcgcggcatgatttcaaagaaaaaaatctcaaagagatcagcgacttaattaagaaggagcttacggagggtaaagcactggggctcagtcatacatacgccaacgtccgcattaatcatgtaattaatctgtggggcgctgacttcgattcgaatggcaatttgaaggcgatctatgtgaccgatagtgacagtaacgcttcgattggcatgaagaaatattttgtcggcgtcaactccgctggcaaagtcgcgatttctgccaaagagatcaaggaggataatattggggctcaagtgttgggtttgttcacattatcaacaggccaagactcttggaaccagacaaattaa

SEQ ID NO: 3 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatcccgtaagcggtgctatagtacaagtgcggtcgtactggcggctgttaccttgttcgcgctcagtgtggatcgtggtgtaatcgcggatagcttttcggcgaaccaggagattcggtattccgaagtcacaccatatcatgtaacgtctgtatggacgaaaggtgtaacgcctccggccaaattcacacagggcgaggacgtattccacgccccatatgtcgccaaccaaggttggtatgatattacgaagacgtttaacggcaaggacgacctgttgtgcggggcggccaccgcagggaacatgttgcattggtggtttgaccaaaacaaagaaaaaatcgaagcctatctgaaaaagcacccagataagcagaaaattatgttcggcgatcaagagctcctcgatgtccggaaagtgattaacacgaagggggatcagacgaatagtgagctgtttaactattttcgcgacaaagctttcccggggttatccgcccgtcgcattggcgtcatgcctgacctcgtacttgacatgttcattaatgggtattatctgaatgtatacaaaacccagacaactgatgtaaatcgcacatatcaggagaaagatcggcggggtggcatttttgatgccgtatttacacgtggtgaccagtccaaattattaactagccgtcatgatttcaaggagaagaacttaaaggaaatctctgatttaatcaaaaaggaattgactgaaggtaaggctttaggccttagccatacttatgcaaatgtgcgtattaaccatgttattaacttgtggggggcagactttgactcgaacggcaatttaaaagccatctatgtcacggacagcgacagcaatgcttcgattggtatgaaaaagtattttgtaggcgtaaattcagcgggtaaagttgctatcagtgccaaagagatcaaggaggacaatatcggcgctcaggttcttggtttattcacgctgagcacaggccaagattcttggaatcaaacaaactaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 4 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatcccgtaagcgctgttactcgaccagtgcggttgtactggctgcggtgaccctttttgccctctcggtggaccgtggggtcattgcagattctttctccgcgaatcaagagattcggtattctgaggtgactccttaccacgtaacatcggtatggactaaaggcgttacgccgcctgctaaattcactcagggtgaggatgtattccatgccccttatgtggctaaccagggttggtacgacatcacgaaaaccttcaatggcaaagacgacctcttatgtggcgcagcgactgcggggaacatgttgcactggtggttcgatcaaaataaagaaaaaattgaggcatatctgaaaaagcaccctgacaagcaaaagattatgttcggcgaccaggaactgcttgacgtgcgtaaggtgatcaacactaaaggggaccaaaccaactccgaactgtttaattatttccgcgacaaagctttccctggtttatcggcacggcggattggcgttatgccggatctggtgttggacatgtttattaatggctattaccttaacgtatacaaaacacaaactacagatgtaaatcgcacatatcaagagaaggaccgtcgtgggggcattttcgatgccgtattcacacgtggggatcaaagtaagcttctgacctcgcggcatgatttcaaagaaaaaaatctcaaagagatcagcgacttaattaagaaggagcttacggagggtaaagcactggggctcagtcatacatacgccaacgtccgcattaatcatgtaattaatctgtggggcgctgacttcgattcgaatggcaatttgaaggcgatctatgtgaccgatagtgacagtaacgcttcgattggcatgaagaaatattttgtcggcgtcaactccgctggcaaagtcgcgatttctgccaaagagatcaaggaggataatattggggctcaagtgttgggtttgttcacattatcaacaggccaagactcttggaaccagacaaattaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 5 - DNA, 1023 bp, Streptococcus pyogenes
agaaaaagatgctattcaacttcagctgtagtattggcagcagtgactttatttgctctatcggtagatcgtggtgttatagcagatagtttttctgctaatcaagagattagatattcggaagtaacaccttatcatgttacttccgtttggaccaaaggagttactcctccagcaaaattcactcaaggcgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaaaaaattgaagcatatctaaaaaaacacccagataaacaaaaaatcatgtttggtgatcaagaattattggatgtaagaaaagttattaataccaaaggtgaccaaacaaatagcgagctttttaattatttccgagataaagctttccccggtttgtcagcacgccgaattggagttatgcctgatcttgttttagatatgtttatcaatggttattacttaaatgtttataagacacagactactgatgtcaatagaacctatcaagagaaagatcgccgaggtggtatttttgacgccgtatttacaagaggtgatcaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatattggtgctcaagtactagggttatttacactttcaacagggcaagatagttggaatcagaccaattaa

SEQ ID NO: 6 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccagaaaaagatgctattcaacttcagctgtagtattggcagcagtgactttatttgctctatcggtagatcgtggtgttatagcagatagtttttctgctaatcaagagattagatattcggaagtaacaccttatcatgttacttccgtttggaccaaaggagttactcctccagcaaaattcactcaaggcgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaaaaaattgaagcatatctaaaaaaacacccagataaacaaaaaatcatgtttggtgatcaagaattattggatgtaagaaaagttattaataccaaaggtgaccaaacaaatagcgagctttttaattatttccgagataaagctttccccggtttgtcagcacgccgaattggagttatgcctgatcttgttttagatatgtttatcaatggttattacttaaatgtttataagacacagactactgatgtcaatagaacctatcaagagaaagatcgccgaggtggtatttttgacgccgtatttacaagaggtgatcaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatattggtgctcaagtactagggttatttacactttcaacagggcaagatagttggaatcagaccaattaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 7 - protein, 340 aa, Artificial Sequence
  Synthetic polypeptide
RKRCYSTSAVVLAAVTLFALSVDRGVIADSFSANQEIRYSEVTPYHVTSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 8 - protein, 340 aa, Artificial Sequence; identical to SEQ ID NO: 7
  Synthetic polypeptide

SEQ ID NO: 9 - protein, 340 aa, Streptococcus pyogenes; identical to SEQ ID NO: 7

SEQ ID NO: 10 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagtcctggcggcggtcactctgttcgcactgagcgttgatcgcggtgtgatcgccgattcttttagcgcgaaccaagaaattcgctacagcgaggtgactccgtaccatgtttgggaaatctggttggacggcaccattccgttttactggtggaccgttaccaaagacatgatctacgtgccgtacatcccaaatatgggcatttacttcctgttcaaaacctttgatggcaaagacgacttactgtgcggtgctgcgaccgcagttatgatgattttttggtggctgatggttaatcgtgactgggtttggtggtatatgaaagtttttccgtttatgatctacatctggaaggacaaccaattgttgctggtcttggtgttggtgatgctgacggcttgggatctgctttacccgcacctgtggcttttcttccgcgacatcgcctttccaggtttgtcggcgcgtcgtatcggcgtgatgccggatttggtactcgctatgtttttcatgggttatctgctgaatgtttataagacccagaccaccgatattgaatataaaccgatgccgagagattggcgtggcggtctcttcctgatgattttctggcttggcgatccgcgtttttatatggtgtatcgtcatgattttaaagagctgaatctggagcagatttctcgtatcatgctgtactggttgttaaaaggtgtggtgctgggtctgagccacacctatgccaacgtgcgcatcaaccacgttattaacgtttgggcaattgtcctggacaaggacaacctgctgcgttatatgttcgttgttgattccgattccgacccgcgctggggtctgttcatctggttcatcgacatcaacaacgcgggtaaggtggcgattagctggaagcagatcgataaggacaacattggcgcacaggtgctcggcctgttcgtgttgctgcaaggtaaggacttatggaatctgacggactaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 11 - DNA, 1023 bp, Artificial Sequence
  Synthetic polynucleotide
aggaaaagatgttattcaacaagtgctgtagtcctggcggcggtcactctgttcgcactgagcgttgatcgcggtgtgatcgccgattcttttagcgcgaaccaagaaattcgctacagcgaggtgactccgtaccatgtttgggaaatctggttggacggcaccattccgttttactggtggaccgttaccaaagacatgatctacgtgccgtacatcccaaatatgggcatttacttcctgttcaaaacctttgatggcaaagacgacttactgtgcggtgctgcgaccgcagttatgatgattttttggtggctgatggttaatcgtgactgggtttggtggtatatgaaagtttttccgtttatgatctacatctggaaggacaaccaattgttgctggtcttggtgttggtgatgctgacggcttgggatctgctttacccgcacctgtggcttttcttccgcgacatcgcctttccaggtttgtcggcgcgtcgtatcggcgtgatgccggatttggtactcgctatgtttttcatgggttatctgctgaatgtttataagacccagaccaccgatattgaatataaaccgatgccgagagattggcgtggcggtctcttcctgatgattttctggcttggcgatccgcgtttttatatggtgtatcgtcatgattttaaagagctgaatctggagcagatttctcgtatcatgctgtactggttgttaaaaggtgtggtgctgggtctgagccacacctatgccaacgtgcgcatcaaccacgttattaacgtttgggcaattgtcctggacaaggacaacctgctgcgttatatgttcgttgttgattccgattccgacccgcgctggggtctgttcatctggttcatcgacatcaacaacgcgggtaaggtggcgattagctggaagcagatcgataaggacaacattggcgcacaggtgctcggcctgttcgtgttgctgcaaggtaaggacttatggaatctgacggactaa

SEQ ID NO: 12 - protein, 340 aa, Artificial Sequence
  Synthetic polypeptide
RKRCYSTSAVVLAAVTLFALSVDRGVIADSFSANQEIRYSEVTPYHVWEIWLDGTIPFYWWTVTKDMIYVPYIPNMGIYFLFKTFDGKDDLLCGAATAVMMIFWWLMVNRDWVWWYMKVFPFMIYIWKDNQLLLVLVLVMLTAWDLLYPHLWLFFRDIAFPGLSARRIGVMPDLVLAMFFMGYLLNVYKTQTTDIEYKPMPRDWRGGLFLMIFWLGDPRFYMVYRHDFKELNLEQISRIMLYWLLKGVVLGLSHTYANVRINHVINVWAIVLDKDNLLRYMFVVDSDSDPRWGLFIWFIDINNAGKVAISWKQIDKDNIGAQVLGLFVLLQGKDLWNLTD

SEQ ID NO: 13 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagttctggctgcggttaccctgttcgccctgagcgttgaccgcggtgtgattgccgactccttcagcgccaatcaagaaatccgttacagcgaagtgactccgtatcacgttatgaccgtttgggtcaaagacgtgatcccgccacaatggtggacccaaactaaggactacattcacgcgccgttttacccgaaccagggctggtatgcgctcttcaaaacgtttaatggtaaggacgacctgctttgcggtgcggcgaccgcaatcgtgatgatttggtggtggctggatgtcaacaaagagaaaatcgaagagtacttgaagaaacatccgcaccgtcagcaaatttggaaagatgatcagctgttgctggacatctgggctgtgattaataccaagtgggatatgacgaacccgcacctgtttatctacttccgcgacattgcgtttccgggtctgtcggctcgtcgcattggtgttatgccggatttggttctgatgatgttcataatgggctatccgctgaatgtttataaaacccagacgaccgatgtggactacaagccgatgccacaagattggcgtggtggtatttttatcgacgtgttctggctgggcgacccgcgttactacttagttagccgtcatgattttaaagagaaaaacttgaaggagatctccgatgtcatcctgaagttattgttggaaggaaagatgctgggtctgtcccatacctatgcgaatgttagaatcaaccacgtgatcaacctgtggggtgcggtgttcgattctaacggcttgctgaaggcaatctatgtgaccgattctgatagcgaccctcgtgatggtatgctcttatatttcgtcaacgtaaacagcgctggcaaggtggcaattagcaccaagccgattgacaaagataatatcggcgcgcaggttctgggcctgttcaccctcgacgtgggcaaagacctgtggaaccagaccaactaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 14 - DNA, 1023 bp, Artificial Sequence
  Synthetic polynucleotide
aggaaaagatgttattcaacaagtgctgtagttctggctgcggttaccctgttcgccctgagcgttgaccgcggtgtgattgccgactccttcagcgccaatcaagaaatccgttacagcgaagtgactccgtatcacgttatgaccgtttgggtcaaagacgtgatcccgccacaatggtggacccaaactaaggactacattcacgcgccgttttacccgaaccagggctggtatgcgctcttcaaaacgtttaatggtaaggacgacctgctttgcggtgcggcgaccgcaatcgtgatgatttggtggtggctggatgtcaacaaagagaaaatcgaagagtacttgaagaaacatccgcaccgtcagcaaatttggaaagatgatcagctgttgctggacatctgggctgtgattaataccaagtgggatatgacgaacccgcacctgtttatctacttccgcgacattgcgtttccgggtctgtcggctcgtcgcattggtgttatgccggatttggttctgatgatgttcataatgggctatccgctgaatgtttataaaacccagacgaccgatgtggactacaagccgatgccacaagattggcgtggtggtatttttatcgacgtgttctggctgggcgacccgcgttactacttagttagccgtcatgattttaaagagaaaaacttgaaggagatctccgatgtcatcctgaagttattgttggaaggaaagatgctgggtctgtcccatacctatgcgaatgttagaatcaaccacgtgatcaacctgtggggtgcggtgttcgattctaacggcttgctgaaggcaatctatgtgaccgattctgatagcgaccctcgtgatggtatgctcttatatttcgtcaacgtaaacagcgctggcaaggtggcaattagcaccaagccgattgacaaagataatatcggcgcgcaggttctgggcctgttcaccctcgacgtgggcaaagacctgtggaaccagaccaactaa

SEQ ID NO: 15 - protein, 340 aa, Artificial Sequence
  Synthetic polypeptide
RKRCYSTSAVVLAAVTLFALSVDRGVIADSFSANQEIRYSEVTPYHVMTVWVKDVIPPQWWTQTKDYIHAPFYPNQGWYALFKTFNGKDDLLCGAATAIVMIWWWLDVNKEKIEEYLKKHPHRQQIWKDDQLLLDIWAVINTKWDMTNPHLFIYFRDIAFPGLSARRIGVMPDLVLMMFIMGYPLNVYKTQTTDVDYKPMPQDWRGGIFIDVFWLGDPRYYLVSRHDFKEKNLKEISDVILKLLLEGKMLGLSHTYANVRINHVINLWGAVFDSNGLLKAIYVTDSDSDPRDGMLLYFVNVNSAGKVAISTKPIDKDNIGAQVLGLFTLDVGKDLWNQTN

SEQ ID NO: 16 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagttttggcggcggttacgctcttcgccctgtcggtagatagaggcgttattgcagactccttcagcgcgaaccaggagatccgctacagcgaagttacgccgtaccacgtgacttccgtgtgggttaaaggcgttacgccgcctcagtggtggacccaaaccgaagacttcttttacgccccatacgtcccgaatcaaggttggtatgatttgaccaagacctttaacggcaaggatgatctgctttgcggtgctgcgaccgcgatcaacatgctgtggtggtggtttgacgtgaacaaagagaagattgaggagtatctcaagaagcatccggacaaacaaaaaatcatgaaagacgaccaagagctgctagacgtgcgcaaggttatcaataccaaatgggatcaaacgaatagccacctgttcctgtattttcgtgatattgcgtttccgggtctgtctgcgcgtcgtatcggagtaatgccggatttggttctgatcatgttcatcatgggttattacctgaatgtttacaaaacgcagaccacggacgtcaaccgtaccccgcaagaaaaggactggcgtggtggcatttttatcgctgtcttcaccttgggtgatccgagcaaatatctgaccagccgtcatgattttaaagaaaagaacctgaaggagattagcgacaccatcaaaaaactgttggaggaaggcaaaatgctgggcctgagccacacctatgctaacgtgcgcattaaccacgtgattaatctgtggggtgcagatttcgactctaatggcctgctcaaggccatctacgtgactgacagcgactctaacccgtcgatcggcatgttgaagtacttcgtgggcgtgaacagcgcgggtaaggtggcgatttccctgaaacagattgatgaagataacatcggtgcacaggttctgggcttgttcaccctggacaccggtcaggatctgtggaatcagaccaactaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 17 - DNA, 1023 bp, Artificial Sequence
  Synthetic polynucleotide
aggaaaagatgttattcaacaagtgctgtagttttggcggcggttacgctcttcgccctgtcggtagatagaggcgttattgcagactccttcagcgcgaaccaggagatccgctacagcgaagttacgccgtaccacgtgacttccgtgtgggttaaaggcgttacgccgcctcagtggtggacccaaaccgaagacttcttttacgccccatacgtcccgaatcaaggttggtatgatttgaccaagacctttaacggcaaggatgatctgctttgcggtgctgcgaccgcgatcaacatgctgtggtggtggtttgacgtgaacaaagagaagattgaggagtatctcaagaagcatccggacaaacaaaaaatcatgaaagacgaccaagagctgctagacgtgcgcaaggttatcaataccaaatgggatcaaacgaatagccacctgttcctgtattttcgtgatattgcgtttccgggtctgtctgcgcgtcgtatcggagtaatgccggatttggttctgatcatgttcatcatgggttattacctgaatgtttacaaaacgcagaccacggacgtcaaccgtaccccgcaagaaaaggactggcgtggtggcatttttatcgctgtcttcaccttgggtgatccgagcaaatatctgaccagccgtcatgattttaaagaaaagaacctgaaggagattagcgacaccatcaaaaaactgttggaggaaggcaaaatgctgggcctgagccacacctatgctaacgtgcgcattaaccacgtgattaatctgtggggtgcagatttcgactctaatggcctgctcaaggccatctacgtgactgacagcgactctaacccgtcgatcggcatgttgaagtacttcgtgggcgtgaacagcgcgggtaaggtggcgatttccctgaaacagattgatgaagataacatcggtgcacaggttctgggcttgttcaccctggacaccggtcaggatctgtggaatcagaccaactaa

SEQ ID NO: 18 - protein, 340 aa, Artificial Sequence
  Synthetic polypeptide
RKRCYSTSAVVLAAVTLFALSVDRGVIADSFSANQEIRYSEVTPYHVTSVWVKGVTPPQWWTQTEDFFYAPYVPNQGWYDLTKTFNGKDDLLCGAATAINMLWWWFDVNKEKIEEYLKKHPDKQKIMKDDQELLDVRKVINTKWDQTNSHLFLYFRDIAFPGLSARRIGVMPDLVLIMFIMGYYLNVYKTQTTDVNRTPQEKDWRGGIFIAVFTLGDPSKYLTSRHDFKEKNLKEISDTIKKLLEEGKMLGLSHTYANVRINHVINLWGADFDSNGLLKAIYVTDSDSNPSIGMLKYFVGVNSAGKVAISLKQIDEDNIGAQVLGLFTLDTGQDLWNQTN

SEQ ID NO: 19 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagttctggcagcggttactctgttcgccctgtcagttgatcgtggcgtgattgctgactcgttctccgcgaaccaagaaatccgttatagcgaagttacgccgtaccacgtgacctccgtgtggaccaagggcgtcaccccgccagcgaaatttacccagggtgaagacgtgttccatgcaccgtatgttgcgaatcaaggttggtatgacattaccaagacctttaacggcaaagatgatctgctttgcggtgccgctaccgcaattaacatgctgcattggtggtttgaccaaaacaaagagaagattgaagcatacctaaagaaacacccggacaagcagaaaatcatgttcggcgatcaagagctgctggacgtccgcaaagtgatcaacaccaagtgggatcagacgaacagcgaattgtttaattatttccgcgataaagcgtttccgggtctgagcgcgcgtcgcattggtgtcatgccggacttggttctggacatgtttatcaacggctattacctgaatgtttataaaactcaaacgaccgacgttaatcgtacctaccaggagaaggaccgccgtggtggtatttttgatgcagtattcaccagaggcgatcaaagcaaactgttgacctctcgtcacgatttcaaggaaaaaaacttgaaggagatcagcgatctgatcaaaaaggagttaaccgaaggtaaggcgctcggtttgagccacacctacgcgaacgttcgtattaaccatgtcattaacctgtggggtgcggacttcgactccaacggtaatttgaaggcgatctacgtgacggattccgatagcaacgccagcattggcatgaaaaaatacttcgtgggtgtgaatagtgctggcaaggtggccatcagcgcgaaggagatcaaagaggacaacatcggcgctcaggttctgggcctgtttactctgagcaccggtcaggattcttggaatcagacgaattaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 20 - DNA, 1023 bp, Artificial Sequence
  Synthetic polynucleotide
aggaaaagatgttattcaacaagtgctgtagttctggcagcggttactctgttcgccctgtcagttgatcgtggcgtgattgctgactcgttctccgcgaaccaagaaatccgttatagcgaagttacgccgtaccacgtgacctccgtgtggaccaagggcgtcaccccgccagcgaaatttacccagggtgaagacgtgttccatgcaccgtatgttgcgaatcaaggttggtatgacattaccaagacctttaacggcaaagatgatctgctttgcggtgccgctaccgcaattaacatgctgcattggtggtttgaccaaaacaaagagaagattgaagcatacctaaagaaacacccggacaagcagaaaatcatgttcggcgatcaagagctgctggacgtccgcaaagtgatcaacaccaagtgggatcagacgaacagcgaattgtttaattatttccgcgataaagcgtttccgggtctgagcgcgcgtcgcattggtgtcatgccggacttggttctggacatgtttatcaacggctattacctgaatgtttataaaactcaaacgaccgacgttaatcgtacctaccaggagaaggaccgccgtggtggtatttttgatgcagtattcaccagaggcgatcaaagcaaactgttgacctctcgtcacgatttcaaggaaaaaaacttgaaggagatcagcgatctgatcaaaaaggagttaaccgaaggtaaggcgctcggtttgagccacacctacgcgaacgttcgtattaaccatgtcattaacctgtggggtgcggacttcgactccaacggtaatttgaaggcgatctacgtgacggattccgatagcaacgccagcattggcatgaaaaaatacttcgtgggtgtgaatagtgctggcaaggtggccatcagcgcgaaggagatcaaagaggacaacatcggcgctcaggttctgggcctgtttactctgagcaccggtcaggattcttggaatcagacgaattaa

SEQ ID NO: 21 - protein, 340 aa, Artificial Sequence
  Synthetic polypeptide
RKRCYSTSAVVLAAVTLFALSVDRGVIADSFSANQEIRYSEVTPYHVTSVWTKGVTPPAKFTQGEDVFHAPYVANQGWYDITKTFNGKDDLLCGAATAINMLHWWFDQNKEKIEAYLKKHPDKQKIMFGDQELLDVRKVINTKWDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 22 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagtactggctgctgttactctgttcgccttaagcgttgaccgcggcgtcatcgcggattcctttagcgcgaaccaagagattcgttatagcgaggtgacgccgtaccacgttgaagaagtttggaccaaaggtgttacgccaccggctaaattcacccagggtgaagacgtcttccatgcaccgtacgttgcaaaccagggctggtatgatatcaccaagacctttaatggtaaagatgatctgctttgcggtgctgcgaccgcaggtaatatgctgcattggtggttcgaccagaataaagagaagattgaagcatatttgaaaaagcacccggacaagcaaaaaatcatgttcggcgatcaagagctgcttgatgtgcgcaaggtgattaacaccaagggcgaccaaaccaattccgaactgtttaactacttccgcgataaggcgtttccgggtttgtcggcgcgtcgtatcggggtgatgccggatttggttctggacatgttcatcaacggctattacctgaatgtgtataaaacccaaaccaccgacgtgaatcgtacctaccaggagaaggacagacgtggcggtatttttgacgccgtgttcacccgtggtgaccaaagcaaactgttgaccagccgtcacgattttaaagagctgaacctgaagcaaatcagcgacatcatccgcaaagagttgctggaaggtaaggccctcggcctgagccatacgtacgcaaacgtgcgtattaaccacgtgatcaacctgtggggtgcggtgttcgactctaatggcttgctcaaggcgatttatgtcactgattctgactccaacgccagcattggcatgaaaaaatactttgttgacatcaacagcgcgggcaagctggcgatctctgcgaaacagattgatgaagataatattggtgcgcaggttctgggtttgtttacactgtcgaccggtcaggattcttggaaccagacgaactaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 23 - DNA, 1023 bp, Artificial Sequence
  Synthetic polynucleotide
aggaaaagatgttattcaacaagtgctgtagtactggctgctgttactctgttcgccttaagcgttgaccgcggcgtcatcgcggattcctttagcgcgaaccaagagattcgttatagcgaggtgacgccgtaccacgttgaagaagtttggaccaaaggtgttacgccaccggctaaattcacccagggtgaagacgtcttccatgcaccgtacgttgcaaaccagggctggtatgatatcaccaagacctttaatggtaaagatgatctgctttgcggtgctgcgaccgcaggtaatatgctgcattggtggttcgaccagaataaagagaagattgaagcatatttgaaaaagcacccggacaagcaaaaaatcatgttcggcgatcaagagctgcttgatgtgcgcaaggtgattaacaccaagggcgaccaaaccaattccgaactgtttaactacttccgcgataaggcgtttccgggtttgtcggcgcgtcgtatcggggtgatgccggatttggttctggacatgttcatcaacggctattacctgaatgtgtataaaacccaaaccaccgacgtgaatcgtacctaccaggagaaggacagacgtggcggtatttttgacgccgtgttcacccgtggtgaccaaagcaaactgttgaccagccgtcacgattttaaagagctgaacctgaagcaaatcagcgacatcatccgcaaagagttgctggaaggtaaggccctcggcctgagccatacgtacgcaaacgtgcgtattaaccacgtgatcaacctgtggggtgcggtgttcgactctaatggcttgctcaaggcgatttatgtcactgattctgactccaacgccagcattggcatgaaaaaatactttgttgacatcaacagcgcgggcaagctggcgatctctgcgaaacagattgatgaagataatattggtgcgcaggttctgggtttgtttacactgtcgaccggtcaggattcttggaaccagacgaactaa

SEQ ID NO: 24 - protein, 340 aa, Artificial Sequence
  Synthetic polypeptide
RKRCYSTSAVVLAAVTLFALSVDRGVIADSFSANQEIRYSEVTPYHVEEVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKELNLKQISDIIRKELLEGKALGLSHTYANVRINHVINLWGAVFDSNGLLKAIYVTDSDSNASIGMKKYFVDINSAGKLAISAKQIDEDNIGAQVLGLFTLSTGQDSWNQTN

SEQ ID NO: 25 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagtactggcagcggtgaccttgtttgcactgtcggttgatcgtggggtcatcgccgattcttttagcgcgaatcaagaaattcgttatagcgaagtgaccccgtaccatgtttggagcgtgtggaccaaaggcgttacgccgccagcgaaattcacgcagggtgaagacgttttccacgcaccgtacgtggcgaaccaaggttggtatgatatcaccaaaacctttaacggcaaggacgatttactttgcggtgcggctaccgcaggtaatatgctgcattggtggtttgatcaaaacaaagagaagattgaagcgtatctgaagaaacacccggataagcagaaaatcatgtttggtgaccaagagctgttagacgtgcgcaaagtcattaacaccaagggcgaccaaacgaacagcgaactgttcaactatttccgcgataaggcatttccgggtttgtctgcccgtcgtattggcgtgatgccggacctggttctggatatgttcatcaacggttattacttgaatgtttataaaacccagaccaccgatgtgaatagaacataccaggagaaggaccgccgtggtggtatttttgacgctgtgttcacgcgtggcgaccaaagcaagctgttgaccagccgtcatgatttcaaagagaagaacctgaaagagatcagcgacataatcctgaaggagctcttggaaggtaaggctctgggcctgtctcacacctacgcgaacgttcgcattaaccacgtgatcaacctgtggggcgcggacttcgactccaatggcttgcttaaggccatctacgttactgactccgactccaacgccagcattggcatgaaaaagtactttgttggtgtcaatagcgcgggtaaagttgcgattagcgcgaaagaaatcaaagaggataacatcggcgctcaggtgctgggcctgttcactctgtcgaccggtcaggattcatggaaccagaccaattaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 26 - DNA, 1023 bp, Artificial Sequence
  Synthetic polynucleotide
aggaaaagatgttattcaacaagtgctgtagtactggcagcggtgaccttgtttgcactgtcggttgatcgtggggtcatcgccgattcttttagcgcgaatcaagaaattcgttatagcgaagtgaccccgtaccatgtttggagcgtgtggaccaaaggcgttacgccgccagcgaaattcacgcagggtgaagacgttttccacgcaccgtacgtggcgaaccaaggttggtatgatatcaccaaaacctttaacggcaaggacgatttactttgcggtgcggctaccgcaggtaatatgctgcattggtggtttgatcaaaacaaagagaagattgaagcgtatctgaagaaacacccggataagcagaaaatcatgtttggtgaccaagagctgttagacgtgcgcaaagtcattaacaccaagggcgaccaaacgaacagcgaactgttcaactatttccgcgataaggcatttccgggtttgtctgcccgtcgtattggcgtgatgccggacctggttctggatatgttcatcaacggttattacttgaatgtttataaaacccagaccaccgatgtgaatagaacataccaggagaaggaccgccgtggtggtatttttgacgctgtgttcacgcgtggcgaccaaagcaagctgttgaccagccgtcatgatttcaaagagaagaacctgaaagagatcagcgacataatcctgaaggagctcttggaaggtaaggctctgggcctgtctcacacctacgcgaacgttcgcattaaccacgtgatcaacctgtggggcgcggacttcgactccaatggcttgcttaaggccatctacgttactgactccgactccaacgccagcattggcatgaaaaagtactttgttggtgtcaatagcgcgggtaaagttgcgattagcgcgaaagaaatcaaagaggataacatcggcgctcaggtgctgggcctgttcactctgtcgaccggtcaggattcatggaaccagaccaattaa

SEQ ID NO: 27 - protein, 340 aa, Artificial Sequence
  Synthetic polypeptide
RKRCYSTSAVVLAAVTLFALSVDRGVIADSFSANQEIRYSEVTPYHVWSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDIILKELLEGKALGLSHTYANVRINHVINLWGADFDSNGLLKAIYVTDSDSNASIGMKKYFVGVNSAGKVAISAKEIKEDNIGAQVLGLFTLSTGQDSWNQTN

SEQ ID NO: 28 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagttctggcggctgtgactctcttcgccttgtctgttgatagaggcgttatcgcggattcatttagcgccaaccaagaaattcgttatagcgaagttactccgtaccacgtgacttccgtgtggaccaagggcgtgaccccaccggcaaaattcacgcaaggtgaagacgtgtttcatgcgccgtatgttgcgaaccagggctggtatgatatcaccaagaccttcaatggcaaagatgatttgctttgcggtgctgctaccgcaggcaacatgctgcattggtggtttgaccagaataaagagaagattgaagaatacttgaagaagcacccggactacattaaaatcatgtttggtgatcaagagctgctggacgtgcgtaaagtgattaacaccaagggagaccaaaccaatagcgaactgttcaactacttccgcgataaagcgtttccgggtctgagcgcgcgtcgcattggcgtgatgccggacttggttctggatatgttcatcatgggttattacctaaatgtctataaaacccagaccaccgatgtgaaccgcacctaccaggagaaggaccgtcgtggtggtatctttatcgcggtgttcacgttaggcgaccaaagcaaactgctcacgagccgtcacgacttcaaggagaagaacctgaaagagatcagcgacctgattaaaaaagagttgaccgaaggtaaggcgctgggcttgtcccatacctacgcaaatgttcgtattaaccacgttatcaacctttggggtgcggacttcgattctaacggtaatctgaaggccatctacgtgacggattctgactccaacgccagcattggcatgaaaaagtattttgttggtgtcaactcggcgggtaaagtagcaattagcgctaaagagatcaaggaggacaacatcggcgcgcaggttctgggtctgttcacgctgtccaccggtcaggatctgtggaatatgaccctgtaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 29 - DNA, 1023 bp, Artificial Sequence
  Synthetic polynucleotide
aggaaaagatgttattcaacaagtgctgtagttctggcggctgtgactctcttcgccttgtctgttgatagaggcgttatcgcggattcatttagcgccaaccaagaaattcgttatagcgaagttactccgtaccacgtgacttccgtgtggaccaagggcgtgaccccaccggcaaaattcacgcaaggtgaagacgtgtttcatgcgccgtatgttgcgaaccagggctggtatgatatcaccaagaccttcaatggcaaagatgatttgctttgcggtgctgctaccgcaggcaacatgctgcattggtggtttgaccagaataaagagaagattgaagaatacttgaagaagcacccggactacattaaaatcatgtttggtgatcaagagctgctggacgtgcgtaaagtgattaacaccaagggagaccaaaccaatagcgaactgttcaactacttccgcgataaagcgtttccgggtctgagcgcgcgtcgcattggcgtgatgccggacttggttctggatatgttcatcatgggttattacctaaatgtctataaaacccagaccaccgatgtgaaccgcacctaccaggagaaggaccgtcgtggtggtatctttatcgcggtgttcacgttaggcgaccaaagcaaactgctcacgagccgtcacgacttcaaggagaagaacctgaaagagatcagcgacctgattaaaaaagagttgaccgaaggtaaggcgctgggcttgtcccatacctacgcaaatgttcgtattaaccacgttatcaacctttggggtgcggacttcgattctaacggtaatctgaaggccatctacgtgacggattctgactccaacgccagcattggcatgaaaaagtattttgttggtgtcaactcggcgggtaaagtagcaattagcgctaaagagatcaaggaggacaacatcggcgcgcaggttctgggtctgttcacgctgtccaccggtcaggatctgtggaatatgaccctgtaa

SEQ ID NO: 30 - protein, 340 aa, Artificial Sequence
  Synthetic polypeptide
RKRCYSTSAVVLAAVTLFALSVDRGVIADSFSANQEIRYSEVTPYHVTSVWTKGVTPPAKFTQGEDVFHAPYVANQGWYDITKTFNGKDDLLCGAATAGNMLHWWFDQNKEKIEEYLKKHPDYIKIMFGDQELLDVRKVINTKGDQTNSELFNYFRDKAFPGLSARRIGVMPDLVLDMFIMGYYLNVYKTQTTDVNRTYQEKDRRGGIFIAVFTLGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNMTL

SEQ ID NO: 31 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagtgctggcagcggttactctgttcgccctgtctgttgatcgtggcgtgatcgccgattcttttagcgcgaatcaagaaattcgttacagcgaagttaccccgtaccatgtgacgtcggtgtggaccaagggtgtcacaccgccagcaaagttcacccaaggcgaagatgtgtttcatgcgccgtacgtggcgaaccagggttggtatgatattaccaagacctttaacggcaaagatgatttgctgtgtggtgcggctaccgcaggaaacatgctgcattggtggttcgattgcaataaagagaagatcgaagcatatctgaagaagcacccggacaaacagaaaatcatgtttggcgaccaagagttgctcgacgtacgtaaagttatcaacaccaaaggcgaccagacgaacagcgaactgttcaactacttccgcgacaaagcgtttccgggtttgtcggcgcgtcgtatcggcgtgatgccggatttggttttggacatgttcatcaacggttattacctgaatgtgtataaaacccagacgaccgatgttaatcgtacttaccaggagaaggaccgcagaggtggtatttttgacgcagtctttacgcgcggtgaccaaagcaaactgttgacctctcgtcacgacttcaaagagaagaacctgaaagagatcagcgacctgattaaaaaggaacttaccgaaggtaaggcgctgggtttaagccacacctatgctaatgtacgcattaaccacgttatcaacctgtggggtgcggatttcgactccaatggcaacctgaaggccatctatgttacggattccgactccaacgccagcattggcatgaaaaaatactttgtcggtgtgaacagcgctggtaaggtcgcgatcagcgcgaaagagattaaggaggacaacattggcgctcaggttctgggcctcttcaccctgagttgcggtcaggattcctggaatcaaaccaattaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 32 - DNA, 1023 bp, Artificial Sequence
  Synthetic polynucleotide
aggaaaagatgttattcaacaagtgctgtagtgctggcagcggttactctgttcgccctgtctgttgatcgtggcgtgatcgccgattcttttagcgcgaatcaagaaattcgttacagcgaagttaccccgtaccatgtgacgtcggtgtggaccaagggtgtcacaccgccagcaaagttcacccaaggcgaagatgtgtttcatgcgccgtacgtggcgaaccagggttggtatgatattaccaagacctttaacggcaaagatgatttgctgtgtggtgcggctaccgcaggaaacatgctgcattggtggttcgattgcaataaagagaagatcgaagcatatctgaagaagcacccggacaaacagaaaatcatgtttggcgaccaagagttgctcgacgtacgtaaagttatcaacaccaaaggcgaccagacgaacagcgaactgttcaactacttccgcgacaaagcgtttccgggtttgtcggcgcgtcgtatcggcgtgatgccggatttggttttggacatgttcatcaacggttattacctgaatgtgtataaaacccagacgaccgatgttaatcgtacttaccaggagaaggaccgcagaggtggtatttttgacgcagtctttacgcgcggtgaccaaagcaaactgttgacctctcgtcacgacttcaaagagaagaacctgaaagagatcagcgacctgattaaaaaggaacttaccgaaggtaaggcgctgggtttaagccacacctatgctaatgtacgcattaaccacgttatcaacctgtggggtgcggatttcgactccaatggcaacctgaaggccatctatgttacggattccgactccaacgccagcattggcatgaaaaaatactttgtcggtgtgaacagcgctggtaaggtcgcgatcagcgcgaaagagattaaggaggacaacattggcgctcaggttctgggcctcttcaccctgagttgcggtcaggattcctggaatcaaaccaattaa

SEQ ID NO: 33 - protein, 340 aa, Artificial Sequence
  Synthetic polypeptide
RKRCYSTSAVVLAAVTLFALSVDRGVIADSFSANQEIRYSEVTPYHVTSVWTKGVTPPAKFTQGEDVFHAPYVANQGWYDITKTFNGKDDLLCGAATAGNMLHWWFDCNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSCGQDSWNQTN

SEQ ID NO: 34 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagtcctggcggcagttactctgttcgccctgtccgtggaccgcggtgtgatcgcggactcctttagcgcgaatcaagagatccgttatagcgaagttactccgtaccatgttacctctgtttggaccaagggtgttactccgccggcaaaattcacgcagggcgaagattgttttcacgctccgtatgtggcgaatcaaggctggtatgatattaccaaaacctttaacggaaaggatgatttgctttgcggtgcggctaccgcaggcaacatgctgcattggtggtttgaccagaataaagagaagattgaagcgtatctgaagaagcacccagacaagcagaaaatcatgttcggtgaccaggagctgttagatgtgcgcaaagtcatcaacaccaaaggcgatcaaaccaactctgaattgttcaactatttccgcgataaagcctttccgggtctctcggcgcgtcgtatcggcgtgatgccggacctggttctggacatgtttatcaacggctactacctgaatgtttataaaacccaaaccaccgacgttaatcgtacctaccaggagaaggaccgtagaggcggtattttcgacgccgtttttacgcgtggtgaccaaagcaaactgttgaccagccgtcacgacttcaaagaaaagaaccttaaggaaatcagcgatctgattaaaaaagagctgaccgaaggtaaggctctgggcttgtcccatacgtacgcgaacgtacgcatcaaccacgtgattaacctgtggggtgctgattttgacagcaacggtaatctgaaagcgatttacgttaccgatagcgattccaacgcgagcattgggatgaaaaagtacttcgtgggtgtcaacagcgcaggcaaggtggccatttcttgcaaagagatcaaggaggacaatatcggcgcgcaggtgctcggtttgttcacgctgtcgaccggtcaggatagctggaatcaaacgaactaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 35 - DNA, 1023 bp, Artificial Sequence
  Synthetic polynucleotide
aggaaaagatgttattcaacaagtgctgtagtcctggcggcagttactctgttcgccctgtccgtggaccgcggtgtgatcgcggactcctttagcgcgaatcaagagatccgttatagcgaagttactccgtaccatgttacctctgtttggaccaagggtgttactccgccggcaaaattcacgcagggcgaagattgttttcacgctccgtatgtggcgaatcaaggctggtatgatattaccaaaacctttaacggaaaggatgatttgctttgcggtgcggctaccgcaggcaacatgctgcattggtggtttgaccagaataaagagaagattgaagcgtatctgaagaagcacccagacaagcagaaaatcatgttcggtgaccaggagctgttagatgtgcgcaaagtcatcaacaccaaaggcgatcaaaccaactctgaattgttcaactatttccgcgataaagcctttccgggtctctcggcgcgtcgtatcggcgtgatgccggacctggttctggacatgtttatcaacggctactacctgaatgtttataaaacccaaaccaccgacgttaatcgtacctaccaggagaaggaccgtagaggcggtattttcgacgccgtttttacgcgtggtgaccaaagcaaactgttgaccagccgtcacgacttcaaagaaaagaaccttaaggaaatcagcgatctgattaaaaaagagctgaccgaaggtaaggctctgggcttgtcccatacgtacgcgaacgtacgcatcaaccacgtgattaacctgtggggtgctgattttgacagcaacggtaatctgaaagcgatttacgttaccgatagcgattccaacgcgagcattgggatgaaaaagtacttcgtgggtgtcaacagcgcaggcaaggtggccatttcttgcaaagagatcaaggaggacaatatcggcgcgcaggtgctcggtttgttcacgctgtcgaccggtcaggatagctggaatcaaacgaactaa

SEQ ID NO: 36 - protein, 340 aa, Artificial Sequence
  Synthetic polypeptide
RKRCYSTSAVVLAAVTLFALSVDRGVIADSFSANQEIRYSEVTPYHVTSVWTKGVTPPAKFTQGEDCFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISCKEIKEDNIGAQVLGLFTLSTGQDSWNQTN

SEQ ID NO: 37 - DNA, 6004 bp, Artificial Sequence
  Synthetic polynucleotide
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggaaaagatgttattcaacaagtgctgtagtacttgcggctgtgaccctgttcgccttgagtgttgatcgcggcgtcatcgccgatagctttagcgcgaaccaagaaatccgttatagcgaagttaccccgtaccacgttacgtcttgttggaccaagggcgtgacgccaccggcaaaattcacgcagggtgaggacgtttttcacgcaccgtacgtggcgaaccaaggttggtatgacatcaccaagacctttaacggcaaagacgatctgctttgcggtgcagcgaccgcaggcaacatgctgcattggtggtttgaccagaataaagaaaagatcgaagcatacctgaagaagcacccggataaacagaaaatcatgttcggcgaccaagagttattggacgttcgtaaggtgatcaacaccaaaggcgaccagactaatagcgaactgttcaattattttcgcgacaaagcgtttccgggtttgtccgctcgtcgcattggcgtcatgccggatctggttctggacatgttcatcaacggttattacttgaatgtttataaaactcaaacgaccgacgtcaatagaacgtaccaggagaaggaccgtcgtggcggcatttttgacgctgtgtttacccgtggcgatcaaagcaaactgttgaccagccgtcatgatttcaaagagaagaacctcaaggagatctccgatctgattaaaaaggagtgcaccgaaggtaaagcgctgggtctgagccatacctacgctaacgtgcgcatcaaccacgtgattaatctgtggggtgccgatttcgattccaacggtaacctgaaggcgatttatgttaccgactcggactctaatgcgagcattggcatgaaaaagtacttcgtgggtgtgaacagcgcgggtaaggtggctattagcgcgaaagaaatcaaggaggacaacattggtgcgcaggttctgggtttgttcactctgtccaccggtcaggattcttggaatcaaaccaactaagaattcccgggtcgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 38 - DNA, 1023 bp, Artificial Sequence
  Synthetic polynucleotide
aggaaaagatgttattcaacaagtgctgtagtacttgcggctgtgaccctgttcgccttgagtgttgatcgcggcgtcatcgccgatagctttagcgcgaaccaagaaatccgttatagcgaagttaccccgtaccacgttacgtcttgttggaccaagggcgtgacgccaccggcaaaattcacgcagggtgaggacgtttttcacgcaccgtacgtggcgaaccaaggttggtatgacatcaccaagacctttaacggcaaagacgatctgctttgcggtgcagcgaccgcaggcaacatgctgcattggtggtttgaccagaataaagaaaagatcgaagcatacctgaagaagcacccggataaacagaaaatcatgttcggcgaccaagagttattggacgttcgtaaggtgatcaacaccaaaggcgaccagactaatagcgaactgttcaattattttcgcgacaaagcgtttccgggtttgtccgctcgtcgcattggcgtcatgccggatctggttctggacatgttcatcaacggttattacttgaatgtttataaaactcaaacgaccgacgtcaatagaacgtaccaggagaaggaccgtcgtggcggcatttttgacgctgtgtttacccgtggcgatcaaagcaaactgttgaccagccgtcatgatttcaaagagaagaacctcaaggagatctccgatctgattaaaaaggagtgcaccgaaggtaaagcgctgggtctgagccatacctacgctaacgtgcgcatcaaccacgtgattaatctgtggggtgccgatttcgattccaacggtaacctgaaggcgatttatgttaccgactcggactctaatgcgagcattggcatgaaaaagtacttcgtgggtgtgaacagcgcgggtaaggtggctattagcgcgaaagaaatcaaggaggacaacattggtgcgcaggttctgggtttgttcactctgtccaccggtcaggattcttggaatcaaaccaactaa

SEQ ID NO: 39 - protein, 340 aa, Artificial Sequence
  Synthetic polypeptide
RKRCYSTSAVVLAAVTLFALSVDRGVIADSFSANQEIRYSEVTPYHVTSCWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKECTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

(19) United States
(12) Patent Application Publication
(10) Pub. No.: US 2024/0271115 A1
(43) Pub. Date: Aug. 15, 2024

(54) METHODS AND COMPOSITIONS FOR DEPLETING ANTIBODIES

(71) Applicant: The University of North Carolina at Chapel Hill, Chapel Hill, NC (US)

(72) Inventors: Haiyan Fu, Durham, NC (US); Tierra Bobo, Mebane, NC (US); Preston Samowitz, Chapel Hill, NC (US); Brian Kuhlman, Chapel Hill, NC (US); David Forrest Thieker, Durham, NC (US)

(21) Appl. No.: 18/570,529

(22) PCT Filed: Jun. 14, 2022
(86) PCT No.: PCT/US2022/033355

§ 371 Date: Dec. 14, 2023

Related U.S. Application Data
(60) Provisional application No. 63/210,694, filed on Jun. 15, 2021.

(51) Int. Cl. C12N 9/52; A61K 38/00; A61K 38/48; A61K 48/00

(52) U.S. Cl. CPC C12N 9/52; A61K 38/4873; A61K 48/0083; C12Y 304/2201; A61K 38/00

(57) ABSTRACT

This invention relates to methods and compositions for inhibiting or depleting antibodies, e.g., total IgG including neutralizing antibodies. In particular, the invention relates to methods of inhibiting or depleting antibodies against a heterologous agent when the heterologous agent is administered to a subject, comprising administering to the subject an effective amount of recombinant or modified Streptococcus pyogenes IgG degrading enzyme (IdeS) prepared from codon-optimized nucleic acids and/or modified nucleic acids, thereby inhibiting or depleting antibodies and inhibiting neutralization of the heterologous agent, e.g., to improve viral vector-mediated gene therapy.

### STATEMENT OF PRIORITY

This application claims the benefit of U.S. Provisional Application Ser. No. 63/210,694, filed Jun. 15, 2021, the entire contents of which are incorporated by reference herein.

### STATEMENT OF GOVERNMENT SUPPORT

This invention was made with government support under Grant No. AI146653 awarded by the National Institutes of Health. The government has certain rights in the invention.

### FIELD OF THE INVENTION

This invention relates to methods and compositions for inhibiting or depleting antibodies, e.g., neutralizing antibodies. In particular, the invention relates to methods of inhibiting or depleting antibodies against a heterologous agent when the heterologous agent is administered to a subject, comprising administering to the subject an effective amount of recombinant Streptococcus pyogenes IgG degrading enzyme (IdeS) prepared from codon-optimized nucleic acids, thereby inhibiting or depleting antibodies and inhibiting neutralization of the heterologous agent, e.g., to improve viral vector-mediated gene therapy.

### BACKGROUND OF THE INVENTION

Adeno-associated virus (AAV) vectors are promising gene delivery tools because of long-term transduction in a broad range of tissues, with demonstrated efficacy and safety after systemic delivery in numerous disease models and in clinical trials, especially for monogenic diseases.¹⁻⁷ The demonstration of trans-blood-brain-barrier neurotropic properties of AAV9^{8,9} has led to significant advancements in AAV gene delivery for diseases with global or broad CNS neuropathies, demonstrating promising clinical potential.⁴⁻⁷

As effective AAV gene therapies become available for clinical application, pre-existing host humoral immunity against AAV poses critical challenges. While having no known pathogenesis, AAV is widespread in humans, and >90% of the population is naturally infected, with a high prevalence of antibodies (Abs) to various AAV serotypes.¹⁰⁻¹² Though AAV2 is the most prevalent, cross-reactivity among different serotypes¹⁰⁻¹³ reduces the potential utility of AAV vectors packaged in alternative serotypes. αAAV-Abs also arise following rAAV gene delivery, making re-administration unfeasible. While neutralizing Abs (nAbs) against specific AAV serotypes is used as a critical exclusion criteria in clinical trials, non-neutralizing aAAV-Abs can also trigger vector clearance.¹⁴ Pre-existing αAAV Abs diminish the efficacy of systemically delivered AAV vectors and broadly limit their application in terms of patient eligibility and vector re-administration. No effective approaches are currently available to overcome pre-existing AAV-Abs, although various clinically relevant strategies have been studied to address this issue, including AAV capsid modification and decoys,¹⁵⁻¹⁸ transient pharmacological immunomodulation,¹⁹⁻²⁴ and plasmapheresis.^{22,25}

IgG degrading enzyme of Streptococcus pyogenes (IdeS) is a cysteine protease identified in group A streptococci,²⁶ where the enzyme inactivates IgG Abs bound to the bacterial surface.²⁷ IdeS specifically cleaves IgG molecules at the lower hinge region of the heavy chain in a multistep process, producing one F(ab′)₂ and one homodimeric Fc fragment.^{26,28-31} Numerous studies have demonstrated rapid and effective IgG degradation by IdeS in animals^{30,32} and in clinical trials in humans,³³⁻³⁵ strongly supporting their therapeutic potential. In a Phase 1 clinical trial (NCT01802697), an IV injection of IdeS cleaved the entire plasma IgG-pool within minutes after dosing, with IgG reaching a nadir 6-24 hr after dosing and then recovering slowly.³³ Importantly, IdeS has a short half-life of 4.9±2.8 h and is mostly eliminated within 24 h after dosing, with rapid but transient IgG removal, without dose limiting toxicity.³³ A recently published study showed that IV administration of IdeS decreased αAAV-Abs and enabled liver gene transfer of IV-delivered rAAV vector in mice and non-human primates.³⁶ See also WO 2020/016318.

The present invention overcomes shortcomings in the art by providing optimized IdeS enzymes with improved stability and effectiveness in vivo.

### SUMMARY OF THE INVENTION

The present invention is based, in part, on the concept that IdeS administration (e.g., intravenous administration) offers an effective approach for transient removal of pre-existing anti-AAV IgG for rAAV-mediated gene therapy, with potential to benefit all patients who need the treatments and patients who may need re-administration after the initial vector treatment. The present invention may be the answer to the challenge posed by pre-existing anti-AAV antibodies to the translation of AAV gene therapy. Further, the invention may be applicable to any heterologous agent for administration to a subject and which may be recognized by antibodies present in the subject. The present invention further relates to the development of codon-optimized IdeS-encoding nucleic acids and the finding that recombinant IdeS expressed from the codon-optimized sequences is more effective at eliminating antibodies than IdeS expressed from wild-type nucleic acids. The present invention further relates to the development of IdeS variants with increased stability and effectiveness.

Thus, one aspect of the invention relates to a recombinant nucleic acid comprising a sequence encoding Streptococcus pyogenes IgG degrading enzyme (IdeS) that is codon-optimized for expression in E. coli cells, wherein the recombinant nucleic acid comprises a nucleotide sequence at least 90% identical to SEQ ID NO:1 (opt2) or SEQ ID NO:2 (opt1), as well as vectors and cells comprising the same.

Another aspect of the invention relates to a recombinant nucleic acid comprising a sequence encoding a modified Streptococcus pyogenes IgG degrading enzyme (IdeS) for expression in E. coli cells, wherein the recombinant nucleic acid comprises a nucleotide sequence identical to SEQ ID NO:11 (mt1), SEQ ID NO:14 (mt2), SEQ ID NO:17 (mt3), SEQ ID NO:20 (mt4), SEQ ID NO:23 (mt5), SEQ ID NO:26 (mt6), SEQ ID NO: 29 (mt7), SEQ ID NO:32 (mt8), SEQ ID NO:35 (mt9), or SEQ ID NO:38 (mt10) or a sequence at least 90% identical thereto, e.g., at least 90%, 91%, 92%, 93%, 94%, 95%, 96%, 97%, 98%, 99%, or 99.5% identical thereto. In some embodiments, the recombinant nucleic acid comprising a sequence encoding the modified IdeS is codon-optimized.

In other embodiments, the modified IdeS comprises an amino acid sequence identical to SEQ ID NO:12 (mt1), SEQ ID NO:15 (mt2), SEQ ID NO:18 (mt3), SEQ ID NO:21 (mt4), SEQ ID NO:24 (mt5), SEQ ID NO:27 (mt6), SEQ ID NO: 30 (mt7), SEQ ID NO:33 (mt8), SEQ ID NO:36 (mt9), or SEQ ID NO:39 (mt10) or a sequence at least 90% identical thereto, e.g., at least 90%, 91%, 92%, 93%, 94%, 95%, 96%, 97%, 98%, 99%, or 99.5% identical thereto. In some embodiments, the modified IdeS comprises a cysteine substitution at two residues to enable disulfide bond formation.

A further aspect of the invention relates to a recombinant or modified IdeS produced from the recombinant nucleic acid or the vector of the invention and a pharmaceutical formulation comprising the same.

Another aspect of the invention relates to a method of producing recombinant or modified IdeS, the method comprising expressing the IdeS from the recombinant nucleic acid or the vector of the invention.

An additional aspect of the invention relates to a method of inhibiting binding of a heterologous agent by antibodies upon administration of the heterologous agent to a subject, comprising administering to the subject an effective amount of the recombinant or modified IdeS or the pharmaceutical formulation of the invention, thereby inhibiting binding of the heterologous agent by antibodies.

A further aspect of the invention relates to a method of expressing a polypeptide or functional nucleic acid in a subject, comprising administering to the subject (a) a nucleic acid delivery vector encoding the polypeptide or functional nucleic acid, and (b) an effective amount of the recombinant or modified IdeS of the invention, thereby expressing the polypeptide or functional nucleic acid in the subject.

Another aspect of the invention relates to a method of editing a gene in a subject, comprising administering to the subject (a) a gene editing complex, and (b) an effective amount of the recombinant or modified IdeS of the invention, thereby expressing the polypeptide or functional nucleic acid in the subject.

An additional aspect of the invention relates to a method of treating an autoimmune disease in a subject in need thereof, comprising administering to the subject an effective amount of the recombinant or modified IdeS of the invention, thereby treating the autoimmune disease.

These and other aspects of the invention are set forth in more detail in the description of the invention below.

### BRIEF DESCRIPTION OF THE DRAWINGS

FIGS. 1A-1B show enhancement of IgG cleavage by recombinant IdeS expressed using codon-optimization. Fresh growth of BC21 cells containing pGEX-6-IdeS, pGEX-6-IdeS^{op1} or pGEX-6-IdeS^{op2} plasmid were incubated at RT for 16 h in 15 ml LB broth containing ampicillin and IPTG (100 μM), each starting at OD₆₀₀ 0.5. Supernatant (media) was concentrated 10-fold using a spin column and cell lysates were processed to purify the rIdeS proteins. Human serum (10 μl) was incubated with media samples (2 μl) or cell lysates (2 μl) at 37° C. for 1 h or 24 h (FIG. 1A), and 20 μl of human serum was incubated for 1 h with the purified IdeS proteins (FIG. 1B), before being analyzed by PAGE/Coomassie blue staining. MW: molecular weight marker; wt: IdeS; op1: IdeS^{op1}; op2: IdeS^{op2}; bc: BC21 cell control. The rIdeS were normalized by protein concentration.

FIG. 2 shows that rapid and effective cleavage of rabbit IgG by Ab-C is dose-dependent. Purified Rb IgG (10 μl) was incubated with 0.03-1.0 μg purified Ab-C at 37° C. for 1 h or 24 h, before PAGE/Coomassie analysis. MW: molecular weight marker; Ab-C: purified IdeS^{op2}.

FIGS. 3A-3B show depletion of αAAV9-IgG by Ab-C in vivo in rabbitized mice. wt C57BL/6 mice were given an IV injection of 100 μl of purified αAAV9-Ab-positive Rb IgG via tail vein. At 10 minutes post IgG infusion, subsets of mice were given an IV injection of IdeS at 0.45 mg/kg (FIGS. 3A, 3B) or 0.225 mg/kg (FIG. 3B). Blood samples were collected at 0 h, 1 h, 4 h, and/or 24 h post Ab-C injection. Serum samples were assayed for total αAAV9-IgG by binding ELISA, and data is expressed as ELISA titer. Control received Rb-IgG only (n=3)(FIG. 3A).

FIGS. 4A-4B show αAAV9-IgG depletion retains the transduction efficiency of IV-delivered scAAV9-hSGSH vector in rabbitized αAAV9-IgG⁺ MPS IIIA mice. 9 MPS IIIA mice were treated with an IV injection of Rb-IgG, of which 5 animals were then given an IV injection of Ab-C (0.25 mg/kg). 5 mice without Rb-IgG were used as controls. All 14 mice were then given an IV injection of scAAV9-hSGSH vector, at the time point of 24 h post Ab-C treatment. Blood draws were performed at 0 h, 1 h, 4 h, and 24 h post Ab-C injection. Necropsy was performed at 1 wk post vector injection. FIG. 4A shows serum samples that were assayed by binding ELISA for αAAV9-IgG. *: p<0.05 vs. 0 h; #: p>0.05 vs. Ab+; ∧: p<0.05 vs. 1 h; +: p<0.05 vs. 4 h. FIG. 4B is total DNA from tissues that was assayed by qPCR for vector genome, expressed as vg/diploid genome (dg).

FIGS. 5A-5B show that Ab-C αAAV9-IgG depletion allow efficient rSGSH expression and clearance of GAG contents in the CNS and periphery tissues in rabbitized αAAV9-IgG⁺ MPS IIIA mice following an IV AAV9-hSGSH vector delivery. MPS IIIA mice (n=4/group) were given an IV injection of Rb-IgG, of which four were then given an IV injection of Ab-C (0.45 mg/kg). MPS IIIA mice without Rb-IgG infusion (n=4) were used as αAAV9-Ab⁻ controls. At 24 h post Ab-C treatment, all 12 mice were then treated with an IV injection of 5×10¹³ vg/kg scAAV9-hSGSH vector. Necropsy was performed at 1-week post vector infusion and tissues were assayed for SGSH activity (FIG. 5A) and GAG contents (FIG. 5B). Tissues from non-treated wild-type (WT) and MPS IIIA mice were used as controls (n=4/group). WT: non-treated WT mice; MPS-NT: non-treated MPS IIIA mice; IgG+: rabbitized αAAV9-IgG⁺ MPS IIIA mice treated with AAV9; IgG+Ab-C: rabbitized αAAV9-IgG⁺ MPS IIIA mice treated with Ab-C and AAV9; IgG−: αAAV9-IgG⁻ MPS IIIA mice treated with AAV9. *: p<0.05 vs. WT; #: p>0.05 vs. WT; ∧: p<0.05 vs. IgG+; +: p<0.05 vs. MPS-NT; @: p>0.05 vs. MPS-NT.

FIG. 6 shows cleavage of rabbit IgG by Ab-C mutant products. Purified rabbit IgG was incubated with modified IdeS proteins at 37° C. for 1 hr and then analyzed by PAGE/Coomassie blue staining. WT: wildtype IdeS; BC: negative bacterial control; mt-1-10: mutated IdeS #1-10.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, N Y, 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4th ed., Lippincott-Raven Publishers).

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4th ed., Lippincott-Raven Publishers); and Table 1.

The term “adeno-associated virus” (AAV) in the context of the present invention includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J Virol. 78:6381-6388 and Table 1), which are also encompassed by the term “AAV.”

The parvovirus particles and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC 001863, NC 001829, NC 001862, NC_000883, NC 001701, NC 001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC 001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999)J. Virol. 73: 939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-:375-383; Mori et al., (2004) Virol. 330:375; Muramatsu et al., (1996) Virol. 221:208; Ruffing et al., (1994) J. Gen. Virol. 75:3385; Rutledge et al., (1998)J. Virol. 72:309; Schmidt et al., (2008)J. Virol. 82:8911; Shade et al., (1986) J. Virol. 58:921; Srivastava et al., (1983) J. Virol. 45:555; Xiao et al., (1999) J Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

A “chimeric” AAV nucleic acid capsid coding sequence or AAV capsid protein is one that combines portions of two or more capsid sequences. A “chimeric” AAV virion or particle comprises a chimeric AAV capsid protein.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

TABLE 1

| | GenBank |
|---|---|
| AAV | Accession |
| Serotypes/Isolates | Number |
| Clonal Isolates | |
| Avian AAV ATCC | AY186198, |
| VR-865 | AY629583, |
| | NC_004828 |
| Avian AAV strain | NC_006263, |
| DA-1 | AY629583 |
| Bovine AAV | NC_005889, |
| | AY388617 |
| AAV4 | NC_001829 |
| AAV5 | AY18065, |
| | AF085716 |
| Rh34 | AY243001 |
| Rh33 | AY243002 |
| Rh32 | AY243003 |
| AAV10 | AY631965 |
| AAV11 | AY631966 |
| AAV12 | DQ813647 |
| AAV13 | EU285562 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

The term “tropism” as used herein refers to preferential but not necessarily exclusive entry of the vector (e.g., virus vector) into certain cell or tissue type(s) and/or preferential but not necessarily exclusive interaction with the cell surface that facilitates entry into certain cell or tissue types, optionally and preferably followed by expression (e.g., transcription and, optionally, translation) of sequences carried by the vector contents (e.g., viral genome) in the cell, e.g., for a recombinant virus, expression of the heterologous nucleotide sequence(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus and/or from a non-integrated episome, as well as any other form which the virus nucleic acid may take within the cell.

The term “tropism profile” refers to the pattern of transduction of one or more target cells, tissues and/or organs. Representative examples of chimeric AAV capsids have a tropism profile characterized by efficient transduction of cells of the central nervous system (CNS) with only low transduction of peripheral organs (see e.g. U.S. Pat. No. 9,636,370 McCown et al., and US patent publication 2017/0360960 Gray et al.). Vectors (e.g., virus vectors, e.g., AAV capsids) expressing specific tropism profiles may be referred to as “tropic” for their tropism profile, e.g., neuro-tropic, liver-tropic, etc.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

As used herein, the terms “contacting,” “introducing” and “administering” are used interchangeably, and refer to a process by which recombinant or modified IdeS of the present invention is delivered to a cell or a subject. The IdeS enzyme may be administered, contacted, or introduced to a subject in a number of ways, including, but not limited to, direct introduction into a cell (i.e., intracellularly) and/or extracellular introduction into a cavity, interstitial space, or into the circulation of the organism.

As used herein, “transduction” of a cell by a virus vector (e.g., an AAV vector) means entry of the vector into the cell and transfer of genetic material into the cell by the incorporation of nucleic acid into the virus vector and subsequent transfer into the cell via the virus vector.

Viral vectors have been used in a wide variety of gene delivery applications in cells, as well as living animal subjects. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J Biol. Chem. 267:963 (1992); Wu et al., J Biol. Chem. 263:14621 (1988); and Hartmut et al., Canadian Patent Application No. 2,012,311, filed Mar. 15, 1990).

Unless indicated otherwise, “efficient transduction” or “efficient tropism,” or similar terms, can be determined by reference to a suitable positive or negative control (e.g., at least about 50%, 60%, 70%, 80%, 85%, 90%, 95% or more of the transduction or tropism, respectively, of a positive control or at least about 110%, 120%, 150%, 200%, 300%, 500%, 1000% or more of the transduction or tropism, respectively, of a negative control).

Similarly, it can be determined if a virus “does not efficiently transduce” or “does not have efficient tropism” for a target tissue, or similar terms, by reference to a suitable control. In particular embodiments, the virus vector does not efficiently transduce (i.e., does not have efficient tropism for) tissues outside the CNS, e.g., liver, kidney, gonads and/or germ cells. In particular embodiments, undesirable transduction of tissue(s) (e.g., liver) is 20% or less, 10% or less, 5% or less, 1% or less, 0.1% or less of the level of transduction of the desired target tissue(s) (e.g., CNS cells).

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and mitochondrial genome, and therefore includes integration of the nucleic acid into, for example, the mitochondrial genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism. Stable transformation of a cell can be detected by, for example, a Northern blot hybridization assay of RNA of the cell with nucleic acid sequences which specifically hybridize with a nucleotide sequence of a transgene introduced into an organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide,” “nucleic acid,” or “nucleotide sequence” may be of RNA, DNA or DNA-RNA hybrid sequences (including both naturally occurring and non-naturally occurring nucleotides), but is preferably either a single or double stranded DNA sequence. The term polynucleotide, nucleotide sequence, or nucleic acid refers to a chain of nucleotides without regard to length of the chain.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. The region in a nucleic acid sequence or polynucleotide in which one or more regulatory elements are found may be referred to as a “regulatory region.”

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence (for example, in a wildtype sequence, including, e.g., a coding sequence for IdeS) with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J Mol. Evol. 35:351 (1987); the method is similar to that described by Higgins & Sharp, CABIOS 5:151 (1989).

Another example of a useful algorithm is the BLAST algorithm, described in Altschul et al., J Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the term “modified,” as applied to a polynucleotide or polypeptide sequence, refers to a sequence that differs from a wildtype sequence due to one or more deletions, additions, substitutions, or any combination thereof.

As used herein, by “isolate” (or grammatical equivalents) a virus vector, it is meant that the virus vector is at least partially separated from at least some of the other components in the starting material.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease, and encompass any type of prophylactic treatment to reduce the incidence of the condition or delays the onset of the condition.

A “treatment effective” or “therapeutically effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

A “heterologous nucleotide sequence” or “heterologous nucleic acid,” with respect to a virus, is a sequence or nucleic acid, respectively, that is not naturally occurring in the virus. Generally, the heterologous nucleic acid or nucleotide sequence comprises an open reading frame that encodes a polypeptide and/or a nontranslated RNA.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the term “vector,” “virus vector,” “delivery vector” (and similar terms) in a specific embodiment generally refers to a virus particle that functions as a nucleic acid delivery vehicle, and which comprises the viral nucleic acid (i.e., the vector genome) packaged within the virion. Virus vectors according to the present invention comprise a chimeric AAV capsid according to the invention and can package an AAV or rAAV genome or any other nucleic acid including viral nucleic acids. Alternatively, in some contexts, the term “vector,” “virus vector,” “delivery vector” (and similar terms) may be used to refer to the vector genome (e.g., vDNA) in the absence of the virion and/or to a viral capsid that acts as a transporter to deliver molecules tethered to the capsid or packaged within the capsid.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged.

A “recombinant AAV vector genome” or “rAAV genome” is an AAV genome (i.e., vDNA) that comprises at least one inverted terminal repeat (e.g., one, two or three inverted terminal repeats) and one or more heterologous nucleotide sequences. rAAV vectors generally retain the 145 base terminal repeat(s) (TR(s)) in cis to generate virus; however, modified AAV TRs and non-AAV TRs including partially or completely synthetic sequences can also serve this purpose. All other viral sequences are dispensable and may be supplied in trans (Muzyczka, (1992) Curr. Topics Microbiol. Immunol. 158:97). The rAAV vector optionally comprises two TRs (e.g., AAV TRs), which generally will be at the 5′ and 3′ ends of the heterologous nucleotide sequence(s), but need not be contiguous thereto. The TRs can be the same or different from each other. The vector genome can also contain a single ITR at its 3′ or 5′ end.

The term “terminal repeat” or “TR” includes any viral terminal repeat or synthetic sequence that forms a hairpin structure and functions as an inverted terminal repeat (ITR) (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or the SV40 hairpin that serves as the origin of SV40 replication can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV terminal repeat” or “AAV TR” may be from any AAV, including but not limited to serotypes 1, 2, 3, 4, 5, 6, 7, 8, 9, 10 or 11 or any other AAV now known or later discovered (see, e.g., Table 1). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

The terms “rAAV particle” and “rAAV virion” are used interchangeably here. A “rAAV particle” or “rAAV virion” comprises a rAAV vector genome packaged within an AAV capsid.

The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

Further, the viral capsid or genomic elements can contain other modifications, including insertions, deletions and/or substitutions.

As used herein, the term “amino acid” encompasses any naturally occurring amino acids, modified forms thereof, and synthetic amino acids, including non-naturally occurring amino acids.

Naturally occurring, levorotatory (L−) amino acids are shown in Table 2.

TABLE 2

| | Abbreviation | |
|---|---|---|
| | Three-Letter | One-Letter |
| Amino Acid Residue | Code | Code |
| Alanine | Ala | A |
| Arginine | Arg | R |
| Asparagine | Asn | N |
| Aspartic acid | Asp | D |
| (Aspartate) | | |
| Cysteine | Cys | C |
| Glutamine | Gln | Q |
| Glutamic acid | Glu | E |
| (Glutamate) | | |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 3) or can be an amino acid that is modified by post-translation modification (e.g., acetylation, amidation, formylation, hydroxylation, methylation, phosphorylation or sulfatation).

TABLE 3
Amino Acid Residue Derivatives

| Modified Amino Acid Residue | Abbreviation |
|---|---|
| 2-Aminoadipic acid | Aad |
| 3-Aminoadipic acid | bAad |
| beta-Alanine, beta- | bAla |
| Aminoproprionic acid | |
| 2-Aminobutyric acid | Abu |
| 4-Aminobutyric acid, | 4Abu |
| Piperidinic acid | |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | alle |
| Methionine sulfoxide | MSO |
| N-Methylglycine, sarcosine | MeGly |
| N-Methylisoleucine | Melle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-C1) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid as described by Wang et al., (2006) Annu. Rev. Biophys. Biomol. Struct. 35:225-49. These unnatural amino acids can advantageously be used to chemically link molecules of interest to the AAV capsid protein.

The term “template” or “substrate” is used herein to refer to a polynucleotide sequence that may be replicated to produce the parvovirus viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in, e.g., FIELDS et al., VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wildtype or synthetic. A wildtype large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another.

Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al., VIROLOGY, volume 2, chapters 69 & 70 (4th ed., Lippincott-Raven Publishers).

By “substantially retain” a property, it is meant that at least about 75%, 85%, 90%, 95%, 97%, 98%, 99% or 100% of the property (e.g., activity or other measurable characteristic) is retained.

### Codon-Optimized Sequences Encoding IdeS

The present invention provides nucleic acids encoding IdeS that are codon-optimized for expression in competent E. coli cells and recombinant IdeS protein produced from the nucleic acids.

One aspect of the invention relates to a recombinant nucleic acid comprising, consisting essentially of, or consisting of a nucleotide sequence encoding Streptococcus pyogenes IgG degrading enzyme (IdeS) that is codon-optimized for expression in bacteria cells. In certain embodiments, the nucleic acid is a non-naturally occurring sequence. In some embodiments, the nucleic acid comprises, consists essentially of, or consists of a nucleotide sequence that is at least 90% identical to SEQ ID NO:1 or SEQ ID NO:2, e.g., at least 91%, 92%, 93%, 94%, 95%, 96%, 97%, 98%, 99%, or 99.5% identical to SEQ ID NO:1 or SEQ ID NO:2. In some embodiments, the nucleic acid comprises, consists essentially of, or consists of the nucleotide sequence of SEQ ID NO:1 or SEQ ID NO:2. In some embodiments, the nucleic acid comprises at least 10 contiguous nucleotides of SEQ ID NO:1 or SEQ ID NO:2, e.g., at least 10, 25, 50, 100, 200, 300, 400, 500, 600, 700, 800, 900, 1000 or more.

Methods of codon optimizing a nucleotide sequence to maximize expression in an organism are well known in the art and can be carried out using software available to the public. The wild-type sequence of the IdeS gene is known in the art and shown in SEQ ID NO:5.

The nucleotide sequence encoding IdeS may be operably linked to a promoter, e.g., a constitutive promoter or an inducible promoter.

The invention also provides a vector comprising the recombinant nucleic acid of the invention, e.g., an expression vector for producing IdeS. In some embodiments, the vector is a plasmid. Examples include, without limitation, the plasmid pGEX-IdeS^{op2} comprising, consisting essentially of, or consisting of SEQ ID NO:3 and the plasmid pGEX-IdeS^{op1} comprising, consisting essentially of, or consisting of SEQ ID NO:4 or a sequence at least 90% identical to SEQ ID NO:3 or SEQ ID NO:4, e.g., at least 91%, 92%, 93%, 94%, 95%, 96%, 97%, 98%, 99%, or 99.5% identical to SEQ ID NO:3 or SEQ ID NO:4.

The invention further provides a cell in vitro comprising the recombinant nucleic acid or vector of the invention, e.g., stably incorporated into the genome of the cell. The cell may be a bacterial cell, e.g., a competent E. coli cell, an insect cell, a plant cell, or a mammalian cell.

Another aspect of the invention relates to a recombinant IdeS protein produced from the recombinant nucleic acid or the vector of the invention. The inventors have surprisingly discovered that recombinant IdeS produced from the codon-optimized sequences of the invention is more effective at degrading antibodies in vitro and in vivo relative to recombinant IdeS produced from wild-type nucleic acid sequences. Without being bound by theory, it is thought that the recombinant IdeS of the invention may be expressed in a form that results in greater expression and/or function of the enzyme.

The recombinant IdeS of the invention may be produced using recombinant expression systems well known in the art and as described herein. The IdeS may be produced, e.g., in bacterial cells. The IdeS may contain a tag that simplifies purification of the expressed protein, e.g., a His tag or a GST tag.

In one aspect of the invention, the recombinant IdeS of the invention includes mutations that result in one or more modifications to the amino acid sequence. In some embodiments, the nucleic acid comprises, consists essentially of, or consists of a nucleotide sequence that is at least 90% identical to SEQ ID NO:23, SEQ ID NO:26, SEQ ID NO:32, SEQ ID NO:35, or SEQ ID NO:38, e.g., at least 91%, 92%, 93%, 94%, 95%, 96%, 97%, 98%, 99%, or 99.5% identical to SEQ ID NO:23, SEQ ID NO:26, SEQ ID NO:32, SEQ ID NO:35, or SEQ ID NO:38. In some embodiments, the nucleic acid comprises, consists essentially of, or consists of the nucleotide sequence of SEQ ID NO:23, SEQ ID NO:26, SEQ ID NO:32, SEQ ID NO:35, or SEQ ID NO:38. In some embodiments, the nucleic acid comprises at least 10 contiguous nucleotides of SEQ ID NO:23, SEQ ID NO:26, SEQ ID NO:32, SEQ ID NO:35, or SEQ ID NO:38, e.g., at least 10, 25, 50, 100, 200, 300, 400, 500, 600, 700, 800, 900, 1000 or more.

In some embodiments, the mutations result in a modification of two amino acids to cysteine residues that are capable of forming a disulfide bond. Without wishing to being bound by theory, it is believed that the modifications, including formation of a disulfide bond, increase the stability of the enzyme and/or the activity of the enzyme, allowing the enzyme to more efficiently inhibit or deplete antibodies against a heterologous agent.

The invention also provides a vector comprising the recombinant nucleic acid of the invention, e.g., an expression vector for producing mutant IdeS. In some embodiments, the vector is a plasmid. Examples include, without limitation, the plasmid pGEX-IdeS^{mt5} comprising, consisting essentially of, or consisting of SEQ ID NO:22, the plasmid pGEX-IdeS^{mt6} comprising, consisting essentially of, or consisting of SEQ ID NO:25, the plasmid pGEX-IdeS^{mt8} comprising, consisting essentially of, or consisting of SEQ ID NO:31, the plasmid pGEX-IdeS^{mt9} comprising, consisting essentially of, or consisting of SEQ ID NO:34, and the plasmid pGEX-IdeS^{mt10} comprising, consisting essentially of, or consisting of SEQ ID NO:37 or a sequence at least 90% identical to SEQ ID NO:22, SEQ ID NO:25, SEQ ID NO:31, SEQ ID NO:34, or SEQ ID NO:37, e.g., at least 91%, 92%, 93%, 94%, 95%, 96%, 97%, 98%, 99%, or 99.5% identical to SEQ ID NO:22, SEQ ID NO:25, SEQ ID NO:31, SEQ ID NO:34, or SEQ ID NO:37.

Another aspect of the invention relates to a recombinant IdeS mutant protein produced from the recombinant nucleic acid or the vector of the invention. The inventors have surprisingly discovered that recombinant mutant IdeS produced from the sequences of the invention is more effective at degrading antibodies in vitro and in vivo relative to recombinant IdeS produced from wild-type nucleic acid sequences. Without being bound by theory, it is thought that the recombinant mutant IdeS of the invention may be expressed in a form that results in greater stability and/or function of the enzyme.

In some embodiments, the recombinant IdeS mutant protein (modified IdeS) comprises, consists essentially of, or consists of an amino acid sequence identical to SEQ ID NO:12 (mt1), SEQ ID NO:15 (mt2), SEQ ID NO:18 (mt3), SEQ ID NO:21 (mt4), SEQ ID NO:24 (mt5), SEQ ID NO:27 (mt6), SEQ ID NO: 30 (mt7), SEQ ID NO:33 (mt8), SEQ ID NO:36 (mt9), or SEQ ID NO:39 (mt10). In other embodiments, the modified IdeS is codon-optimized for expression in E. coli cells.

### Methods of Inhibiting Antibodies

One aspect of the present invention relates to a method of inhibiting or depleting antibodies (e.g., IgG) in a subject, comprising administering to the subject an effective amount of the recombinant IdeS of the present invention or produced by the methods of the present invention, thereby inhibiting or depleting antibodies.

One aspect of the present invention relates to a method of inhibiting inactivation or neutralization of a heterologous agent by antibodies (e.g., neutralizing antibodies) upon administration of the heterologous agent to a subject, comprising administering to the subject an effective amount of the recombinant IdeS of the present invention or produced by the methods of the present invention, thereby inhibiting inactivation or neutralization of the heterologous agent.

Another aspect of the invention relates to a method of expressing a polypeptide or functional nucleic acid in a subject, comprising administering to the subject (a) a nucleic acid delivery vector encoding the polypeptide or functional nucleic acid, and (b) an effective amount of the recombinant IdeS of the present invention or produced by the methods of the present invention, thereby expressing the polypeptide or functional nucleic acid in the subject.

A further aspect of the invention relates to a method of editing a gene in a subject, comprising administering to the subject (a) a gene editing complex, and (b) an effective amount of the recombinant IdeS of the present invention or produced by the methods of the present invention, thereby expressing the polypeptide or functional nucleic acid in the subject.

An additional aspect of the invention relates to a method of treating an autoimmune disease in a subject in need thereof, comprising administering to the subject an effective amount of the recombinant IdeS of the present invention or produced by the methods of the present invention, thereby treating the autoimmune disease.

As used herein, the term “heterologous agent” refers to an agent that is not naturally found in the subject to which the agent is to be administered. The heterologous agent may be one for which antibodies (e.g., neutralizing antibodies) are present in the subject prior to administration of the heterologous agent or one that is likely to raise antibodies (e.g., neutralizing antibodies) upon administration to the subject. The heterologous agent may be one that has never been administered to the subject. The heterologous agent may be one that previously has been administered to the subject.

As used herein, the term “neutralizing antibodies” refers to antibodies that specifically bind to a heterologous agent and inhibit one or more biological activities of the heterologous agent after it has been administered to a subject.

In some embodiments, the heterologous agent may be a nucleic acid delivery vector, e.g., a viral vector or a non-viral vector. In some embodiments, the viral vector is an adeno-associated virus, retrovirus, lentivirus, poxvirus, alphavirus, baculovirus, vaccinia virus, herpes virus, Epstein-Barr virus, or adenovirus vector. In some embodiments, the non-viral vector is a plasmid, liposome, electrically charged lipid, nucleic acid-protein complex, or biopolymer.

In some embodiments, the heterologous agent is a gene editing complex, e.g., a CRISPR complex.

In some embodiments, the heterologous agent is a protein or nucleic acid. In some embodiments, the protein is an enzyme, a regulatory protein, or a structural protein, e.g., one that can substitute for a missing or defective protein in a subject. In some embodiments, the nucleic acid is a functional nucleic acid, e.g., an antisense nucleic acid or an inhibitory RNA.

The effective amount of recombinant IdeS is an amount that at least partially blocks the inhibition of the heterologous agent by antibodies. In some embodiments, the effective amount of recombinant IdeS is an amount sufficient to inhibit inactivation or neutralization by at least about 50%, 60%, 70%, 80%, 90%, 91%, 92%, 93%, 94%, 95%, 96%, 97%, 98, 99%, 99.5%, or 99.9%.

The recombinant IdeS may be administered to the subject by any schedule found to be effective to block inhibition of the heterologous agent by antibodies. In some embodiments, the recombinant IdeS is administered to the subject prior to administration of the heterologous agent, e.g., at least about 1, 5, 10, 15, 20, 30, 40, or 50 minutes or at least about 1, 2, 3, 4, 5, 6, 12, 18, or 24 hours prior to administration of the heterologous agent. In some embodiments, the recombinant IdeS is administered to the subject concurrently with administration of the heterologous agent. As used herein, the term “concurrently” means sufficiently close in time to produce a combined effect (that is, concurrently can be simultaneously, or it can be two or more events occurring within a short time period before or after each other).

In some embodiments, the heterologous agent is combined with the recombinant IdeS prior to administration to the subject, e.g., the two components are mixed together prior to administration in a single composition. In other embodiments, the recombinant IdeS and the heterologous agent are administered in separate compositions.

The recombinant IdeS may be administered to the subject by any route of administration found to be effective to block inhibition of the heterologous agent by antibodies. The most suitable route will depend on the subject being treated and the disorder or condition being treated. In some embodiments, the recombinant IdeS is administered to the subject by a route selected from oral, rectal, transmucosal, intranasal, inhalation (e.g., via an aerosol), buccal (e.g., sublingual), vaginal, intrathecal, intraocular, intravitreal, intracochlear, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain). In some embodiments, the recombinant IdeS is administered to the subject by more than one route, e.g., intravenously and intrathecally.

In some embodiments, the heterologous agent and the recombinant IdeS are administered by the same route. In other embodiments, the heterologous agent and the recombinant IdeS are administered by different routes, e.g., the recombinant IdeS is administered intravenously and the heterologous agent is administered locally to a target tissue or organ.

The recombinant IdeS may be delivered or targeted to any tissue or organ in the subject. In some embodiments, the recombinant IdeS is administered to, e.g., a skeletal muscle, a smooth muscle, the heart, the diaphragm, the airway epithelium, the liver, the kidney, the spleen, the pancreas, the skin, the lung, the ear, and the eye. In some embodiments, the recombinant IdeS is administered to a diseased tissue or organ, e.g., a tumor.

In some amendments, the recombinant IdeS is a derivative of the wild-type amino acid sequence. As used herein, the term “derivative” is used to refer to a polypeptide which differs from a naturally occurring IdeS by minor modifications to the naturally occurring polypeptide, but which significantly retains a biological activity of IdeS. Minor modifications include, without limitation, changes in one or a few amino acid side chains, changes to one or a few amino acids (including deletions, insertions, and/or substitutions), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol. The term “substantially retains,” as used herein, refers to a fragment, derivative, or other variant of a polypeptide that retains at least about 20% of the activity of the naturally occurring polypeptide (e.g., binding to an antibody), e.g., about 30%, 40%, 50% or more. In some embodiments, the derivative of IdeS contains mutations (deletions, insertions, and/or substitutions in any combination) of 10 or fewer amino acid residues, e.g., 10, 9, 8, 7, 6, 5, 4, 3, or 2 or fewer mutations. In some embodiments, the IdeS derivative comprises an amino acid sequence that is at least about 80%, 85%, 90%, 91%, 92%, 93%, 94%, 95%, 96%, 97%, 98%, or 99% identical to the wild-type amino acid sequence of IdeS^{ori} (SEQ ID NO:9).

In some embodiments, the recombinant IdeS can be modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases. Such blocking agents can include, without limitation, additional related or unrelated peptide sequences that can be attached to the amino and/or carboxyl terminal residues of the protein to be administered. This can be done either chemically during the synthesis of the protein or by recombinant DNA technology by methods familiar to artisans of average skill. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the proteins can be covalently or noncovalently coupled to pharmaceutically acceptable “carrier” proteins prior to administration.

### Heterologous Agents

As described above, the heterologous agent may be one for which antibodies (e.g., neutralizing antibodies) are present in the subject prior to administration of the heterologous agent or one that is likely to raise antibodies upon administration to the subject. In some embodiments, the heterologous agent may be a nucleic acid delivery vector (e.g., a viral vector or a non-viral vector), a gene editing complex (e.g., a CRISPR complex), a protein, or a nucleic acid.

Any nucleic acid sequence(s) of interest may be delivered in the nucleic acid delivery vectors of the present invention. Nucleic acids of interest include nucleic acids encoding polypeptides, including therapeutic (e.g., for medical or veterinary uses), immunogenic (e.g., for vaccines), or diagnostic polypeptides.

Therapeutic polypeptides include, but are not limited to, cystic fibrosis transmembrane regulator protein (CFTR), dystrophin (including mini- and micro-dystrophins (see, e.g, Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor −3 and −4, brain-derived neurotrophic factor, bone morphogenic proteins [including RANKL and VEGF], glial derived growth factor, transforming growth factor −α and −β, and the like), lysosomal acid α-glucosidase, α-galactosidase A, receptors (e.g., the tumor necrosis growth factorα soluble receptor), S100A1, parvalbumin, adenylyl cyclase type 6, a molecule that effects G-protein coupled receptor kinase type 2 knockdown such as a truncated constitutively active bARKct, anti-inflammatory factors such as IRAP, anti-myostatin proteins, aspartoacylase, and monoclonal antibodies (including single chain monoclonal antibodies; an exemplary Mab is the Herceptin® Mab). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. Parvovirus vectors can also be used to deliver monoclonal antibodies and antibody fragments, for example, an antibody or antibody fragment directed against myostatin (see, e.g., Fang et al., Nature Biotechnol. 23:584-590 (2005)).

Nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Alternatively, in particular embodiments of this invention, the nucleic acid may encode a functional nucleic acid, i.e., nucleic acid that functions without getting translated into a protein, e.g., an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), RNAi to a sarcoglycan [e.g., α, β, γ], RNAi against myostatin, myostatin propeptide, follistatin, or activin type II soluble receptor, RNAi against anti-inflammatory polypeptides such as the Ikappa B dominant mutant, and RNAi directed against pathogenic organisms and viruses (e.g., hepatitis B virus, human immunodeficiency virus, CMV, herpes simplex virus, human papilloma virus, etc.).

Alternatively, in particular embodiments of this invention, the nucleic acid may encode protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, enos, inos, or bone morphogenic proteins (including BNP 2, 7, etc., RANKL and/or VEGF).

The nucleic acid delivery vectors may also comprise a nucleic acid that shares homology with and recombines with a locus on a host chromosome. This approach can be utilized, for example, to correct a genetic defect in the host cell.

The present invention also provides nucleic acid delivery vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. Nos. 5,882,652, 5,863,541 to Samulski et al.). The antigen may be presented in the parvovirus capsid. Alternatively, the antigen may be expressed from a nucleic acid introduced into a recombinant vector genome. Any immunogen of interest as described herein and/or as is known in the art can be provided by the nucleic acid delivery vectors.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases.

For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Nat. Acad. Sci. USA 91:3515; Kawakami et al., (1994)J Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

It will be understood by those skilled in the art that the nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The nucleic acid delivery vectors provide a means for delivering nucleic acids into a broad range of cells, including dividing and non-dividing cells. The nucleic acid delivery vectors can be employed to deliver a nucleic acid of interest to a cell in vitro, e.g., for ex vivo gene therapy. The nucleic acid delivery vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The nucleic acid delivery vectors can also be used to produce a polypeptide of interest or functional RNA in a subject (e.g., using the subject as a bioreactor to produce the polypeptide or to observe the effects of the functional nucleic acid on the subject, for example, in connection with screening methods).

In general, the nucleic acid delivery vectors of the present invention can be employed to deliver a nucleic acid encoding a polypeptide or functional nucleic acid to treat and/or prevent any disease state for which it is beneficial to deliver a therapeutic polypeptide or functional nucleic acid. Illustrative disease states include, but are not limited to: cystic fibrosis (cystic fibrosis transmembrane regulator protein) and other diseases of the lung, hemophilia A (Factor VIII), hemophilia B (Factor IX), thalassemia (ß-globin), anemia (erythropoietin) and other blood disorders, Alzheimer's disease (GDF; neprilysin), multiple sclerosis (ß-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic defects, congenital emphysema (α1-antitrypsin), Lesch-Nyhan Syndrome (hypoxanthine guanine phosphoribosyl transferase), Niemann-Pick disease (sphingomyelinase), Tays Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, nucleic acid delivery vectors permit the treatment and/or prevention of genetic diseases.

The nucleic acid delivery vectors may also be employed to provide a functional nucleic acid to a cell in vitro or in vivo. Expression of the functional nucleic acid in the cell, for example, can diminish expression of a particular target protein by the cell. Accordingly, functional nucleic acid can be administered to decrease expression of a particular protein in a subject in need thereof.

Nucleic acid delivery vectors find use in diagnostic and screening methods, whereby a nucleic acid of interest is transiently or stably expressed in a transgenic animal model.

The nucleic acid delivery vectors can also be used for various non-therapeutic purposes, including but not limited to use in protocols to assess gene targeting, clearance, transcription, translation, etc., as would be apparent to one skilled in the art. The nucleic acid delivery vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

As a further aspect, the nucleic acid delivery vectors of the present invention may be used to produce an immune response in a subject. According to this embodiment, a nucleic acid delivery vectors comprising a nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the nucleic acid delivery vectors may be administered to a cell ex vivo and the altered cell is administered to the subject. The nucleic acid delivery vectors comprising the nucleic acid is introduced into the cell, and the cell is administered to the subject, where the nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

In particular embodiments, the nucleic acid delivery vector or cell comprising the nucleic acid can be administered in an immunogenically effective amount, as described below.

The nucleic acid delivery vectors can also be administered for cancer immunotherapy by administration of a nucleic acid delivery vector expressing one or more cancer cell antigens (or an immunologically similar molecule) or any other immunogen that produces an immune response against a cancer cell. To illustrate, an immune response can be produced against a cancer cell antigen in a subject by administering a nucleic acid delivery vectors comprising a nucleic acid encoding the cancer cell antigen, for example to treat a patient with cancer and/or to prevent cancer from developing in the subject. The nucleic acid delivery vectors may be administered to a subject in vivo or by using ex vivo methods, as described herein. Alternatively, the cancer antigen can be expressed as part of the nucleic acid delivery vectors.

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with a nucleic acid delivery vectors. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector.

Cytokines may be administered by any method known in the art. Exogenous cytokines may be administered to the subject, or alternatively, a nucleic acid encoding a cytokine may be delivered to the subject using a suitable vector, and the cytokine produced in vivo.

### Subjects, Pharmaceutical Formulations, and Modes of Administration

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a polynucleotide including those described herein. As a further option, the subject can be a laboratory animal and/or an animal model of disease. Preferably, the subject is a human.

In certain embodiments, the heterologous agent and recombinant IdeS are administered to a subject in need thereof as early as possible in the life of the subject, e.g., as soon as the subject is diagnosed with a disease or disorder. In some embodiments, the method are carried out on a newborn subject, e.g., after newborn screening has identified a disease or disorder. In some embodiments, methods are carried out on a subject prior to the age of 10 years, e.g., prior to 1, 2, 3, 4, 5, 6, 7, 8, 9, or 10 years of age. In some embodiments, the methods are carried out on juvenile or adult subjects after the age of 10 years. In some embodiments, the methods are carried out on a fetus in utero, e.g., after prenatal screening has identified a disease or disorder. In some embodiments, the methods are carried out on a subject as soon as the subject develops symptoms associated with a disease or disorder. In some embodiments, the methods are carried out on a subject before the subject develops symptoms associated with a disease or disorder, e.g., a subject that is suspected or diagnosed as having a disease or disorder but has not started to exhibit symptoms.

In particular embodiments, the present invention provides one or more pharmaceutical compositions comprising a heterologous agent and recombinant IdeS in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro, e.g., as part of an ex vivo method. The heterologous agent (e.g., nucleic acid delivery vector, e.g., viral vector) may be introduced into the cells at the appropriate amount, e.g., multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, more preferably at least about 10³ infectious units are introduced to the cell.

The cell(s) into which the nucleic acid delivery vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

The nucleic acid delivery vectors can be introduced into cells in vitro for the purpose of administering the modified cell to a subject. In particular embodiments, the cells have been removed from a subject, the nucleic acid delivery vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the nucleic acid delivery vectors can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the nucleic acid delivery vector are administered to the subject in a treatment effective or prevention effective amount in combination with a pharmaceutical carrier.

In some embodiments, the nucleic acid delivery vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

A further aspect of the invention is a method of administering the heterologous agent (e.g., nucleic acid delivery vector) to subjects. Administration of the nucleic acid delivery vectors to a human subject or an animal in need thereof can be by any means known in the art. Optionally, the nucleic acid delivery vector is delivered in a treatment effective or prevention effective dose in a pharmaceutically acceptable carrier.

The nucleic acid delivery vectors can further be administered to elicit an immunogenic response (e.g., as a vaccine). Typically, immunogenic compositions of the present invention comprise an immunogenically effective amount of nucleic acid delivery vector in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the nucleic acid delivery vector (e.g., viral vector) to be administered to a subject depend upon the mode of administration, the disease or condition to be treated and/or prevented, the individual subject's condition, the particular nucleic acid delivery vector, and the nucleic acid to be delivered, and the like, and can be determined in a routine manner.

Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units, optionally about 10⁸-10¹⁵ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

Administration can also be to a tumor (e.g., in or near a tumor or a lymph node). The most suitable route in any given case will depend on the nature and severity of the condition being treated and/or prevented and on the nature of the particular vector that is being used.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator intermus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronatorteres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, stemohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The heterologous agent can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the heterologous agent is administered to a limb (arm and/or leg) of a subject (e.g., a subject with muscular dystrophy such as DMD) by limb perfusion, optionally isolated limb perfusion (e.g., by intravenous or intra-articular administration. In embodiments of the invention, the heterologous agent can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the agent to cross the endothelial cell barrier. In particular embodiments, the heterologous agent can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Administration to cardiac muscle includes administration to the left atrium, right atrium, left ventricle, right ventricle and/or septum. The heterologous agent can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Administration to smooth muscle can be by any suitable method including intravenous administration, intra-arterial administration, and/or intra-peritoneal administration. In one embodiment, administration can be to endothelial cells present in, near, and/or on smooth muscle.

Delivery to a target tissue can also be achieved by delivering a depot comprising the heterologous agent. In representative embodiments, a depot comprising the heterologous agent is implanted into skeletal, smooth, cardiac and/or diaphragm muscle tissue or the tissue can be contacted with a film or other matrix comprising the heterologous agent. Such implantable matrices or substrates are described in U.S. Pat. No. 7,201,898.

In particular embodiments, a heterologous agent is administered to skeletal muscle, diaphragm muscle and/or cardiac muscle (e.g., to treat and/or prevent muscular dystrophy or heart disease [for example, PAD or congestive heart failure]).

In representative embodiments, the invention is used to treat and/or prevent disorders of skeletal, cardiac and/or diaphragm muscle.

In a representative embodiment, the invention provides a method of treating and/or preventing muscular dystrophy in a subject in need thereof, the method comprising: administering a treatment or prevention effective amount of a heterologous agent to a mammalian subject, wherein the heterologous agent comprises a nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the heterologous agent can be administered to skeletal, diaphragm and/or cardiac muscle as described elsewhere herein.

Alternatively, the invention can be practiced to deliver a nucleic acid to skeletal, cardiac or diaphragm muscle, which is used as a platform for production of a polypeptide (e.g., an enzyme) or functional nuclei acid (e.g., functional RNA, e.g., RNAi, microRNA, antisense RNA) that normally circulates in the blood or for systemic delivery to other tissues to treat and/or prevent a disorder (e.g., a metabolic disorder, such as diabetes (e.g., insulin), hemophilia (e.g., Factor IX or Factor VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid α-glucosidase] or Fabry disease [α-galactosidase A]) or a glycogen storage disorder (such as Pompe disease [lysosomal acid α glucosidase]). Other suitable proteins for treating and/or preventing metabolic disorders are described above. The use of muscle as a platform to express a nucleic acid of interest is described in U.S. Patent Publication No. 2002/0192189.

Thus, as one aspect, the invention further encompasses a method of treating and/or preventing a metabolic disorder in a subject in need thereof, the method comprising: administering a treatment or prevention effective amount of a heterologous agent to a subject (e.g., to skeletal muscle of a subject), wherein the heterologous agent comprises a nucleic acid encoding a polypeptide, wherein the metabolic disorder is a result of a deficiency and/or defect in the polypeptide. Illustrative metabolic disorders and nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory of the invention, according to this embodiment, administration to the skeletal muscle can result in secretion of the polypeptide into the systemic circulation and delivery to target tissue(s). Methods of delivering heterologous agent to skeletal muscle are described in more detail herein.

The invention can also be practiced to produce antisense RNA, RNAi or other functional RNA (e.g., a ribozyme) for systemic delivery.

The invention also provides a method of treating and/or preventing congenital heart failure or PAD in a subject in need thereof, the method comprising administering a treatment or prevention effective amount of a heterologous agent of the invention to a mammalian subject, wherein the heterologous agent comprises a nucleic acid encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active bARKct, Pim-1, PGC-1α, SOD-1, SOD-2, EC-SOD, kallikrein, HIF, thymosin-β4, mir-1, mir-133, mir-206 and/or mir-208.

Injectables can be prepared in conventional forms, either as liquid solutions or suspensions, solid forms suitable for solution or suspension in liquid prior to injection, or as emulsions. Alternatively, one may administer the heterologous agent in a local rather than systemic manner, for example, in a depot or sustained-release formulation. Further, the heterologous agent can be delivered adhered to a surgically implantable matrix (e.g., as described in U.S. Patent Publication No. 2004-0013645).

The heterologous agent disclosed herein can be administered to the lungs of a subject by any suitable means, optionally by administering an aerosol suspension of respirable particles comprised of the heterologous agent, which the subject inhales. The respirable particles can be liquid or solid. Aerosols of liquid particles comprising the heterologous agent may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the heterologous agent may likewise be produced with any solid particulate medicament aerosol generator, by techniques known in the pharmaceutical art.

The heterologous agent can be administered to tissues of the CNS (e.g., brain, eye) and may advantageously result in broader distribution of the heterologous agent than would be observed in the absence of the present invention.

In particular embodiments, the heterologous agent may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, mucopolysaccharidosis (MPS) disorders, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The heterologous agent of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of a heterologous agent encoding one or more neurotrophic factors.

Age-related macular degeneration involves both angiogenesis and retinal degeneration. This disorder can be treated by administering a heterologous agent encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the heterologous agent. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using a heterologous agent of the invention to treat a pituitary tumor.

According to this embodiment, the heterologous agent encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art.

In particular embodiments, the heterologous agent can comprise a secretory signal as described in U.S. Pat. No. 7,071,172.

In representative embodiments of the invention, the heterologous agent is administered to the CNS (e.g., to the brain or to the eye). The heterologous agent may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The heterologous agent may also be administered to different regions of the eye such as the retina, cornea and/or optic nerve.

The heterologous agent may be delivered into the cerebrospinal fluid (e.g., by lumbar puncture) for more disperse administration of the heterologous agent. The heterologous agent may further be administered intravascularly to the CNS in situations in which the blood-brain barrier has been perturbed (e.g., brain tumor or cerebral infarct).

The heterologous agent can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the heterologous agent is administered in a liquid formulation by direct injection (e.g., stereotactic injection) to the desired region or compartment in the CNS. In other embodiments, the heterologous agent may be provided by topical application to the desired region or by intra-nasal administration of an aerosol formulation. Administration to the eye, may be by topical application of liquid droplets. As a further alternative, the heterologous agent may be administered as a solid, slow-release formulation (see, e.g., U.S. Pat. No. 7,201,898).

In yet additional embodiments, the heterologous agent can used for retrograde transport to treat and/or prevent diseases and disorders involving motor neurons (e.g., amyotrophic lateral sclerosis (ALS); spinal muscular atrophy (SMA), etc.). For example, the heterologous agent can be delivered to muscle tissue from which it can migrate into neurons.

Having described the present invention, the same will be explained in greater detail in the following examples, which are included herein for illustration purposes only, and which are not intended to be limiting to the invention.

### EXAMPLES

### Example 1: Development of a New Recombinant IdeS Product, Ab-C, to Overcome the Pre-Existing AAV-Abs for rAAV Gene Delivery

To assess the therapeutic potential of IdeS for AAV gene therapy for MPS IIIA, anew expression construct was developed in order to produce IdeS products for preclinical studies, because there is no IdeS available for in vivo use.

Construction of effective IdeS expression plasmids Three IdeS expression plasmids were made containing 1) the wt IdeS gene cDNA, 2) and 3) a codon-optimized IdeS cDNA each (IdeS^{op1}, IdeS^{op2}). The IdeS cDNAs were cloned to the bacterial expression plasmid pGEX-6P-1, immediate downstream of a GST-tag-HRV3C cleavage site, to generate pGEX-IdeS, pGEX-IdeS^{op1} and pGEX-IdeS^{op2}. The confirmed plasmids were transformed into BL21 competent E. coli cells for high-level expression to produce recombinant IdeS products.

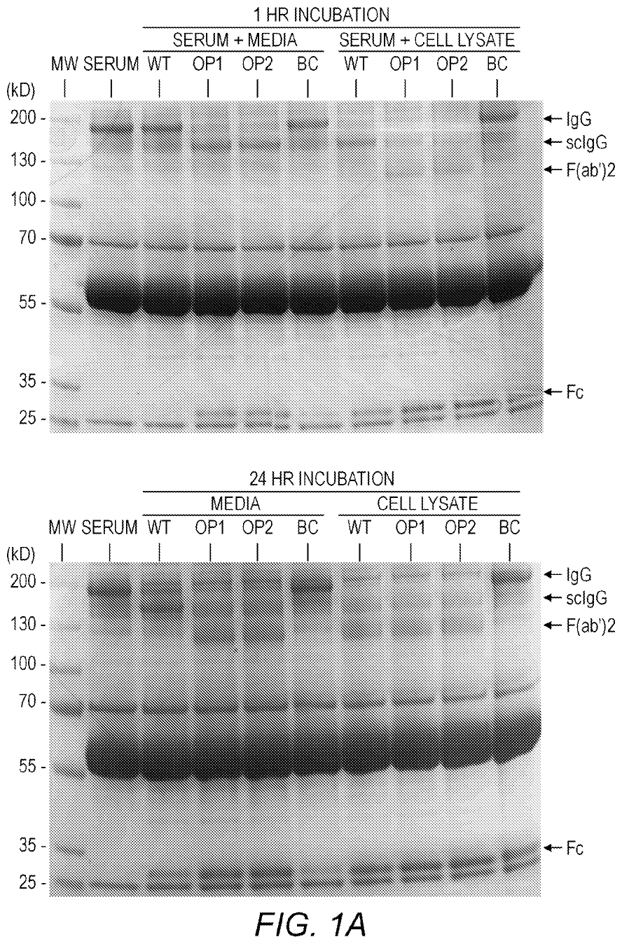

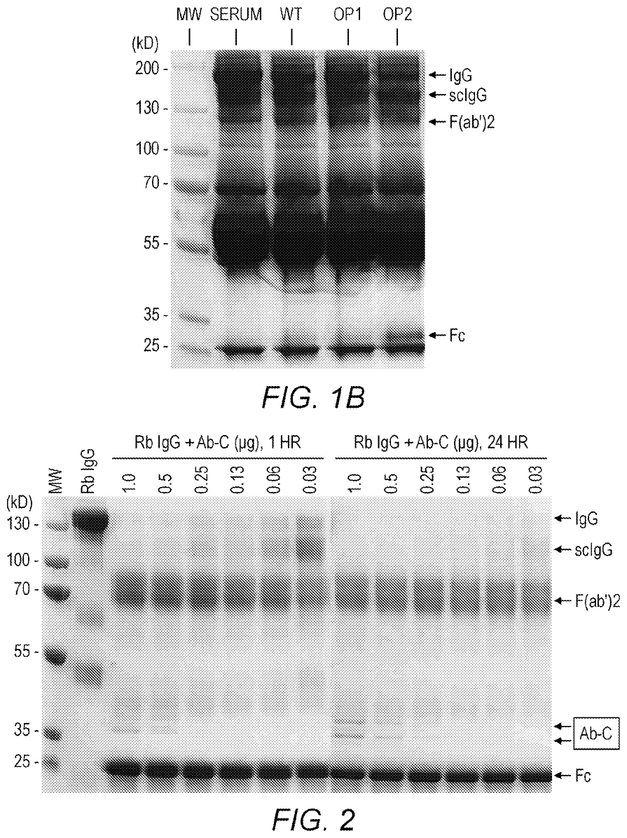

To assess the expression and functionality of the recombinant IdeS by the 3 confirmed IdeS plasmids, the plasmid-containing BL21 cells were incubated overnight at 37° C. in LB containing ampicillin. The cultures were then diluted 1:100 into 15 ml LB broth with ampicillin and incubated at 37° C. for 2-3 h until the OD₆₀₀ reached 0.4-0.8, and 100 μM IPTG was then added to the cultures for 16 h at room temperature (RT). The normalized cell lysates and media samples were incubated at 37° C. with human serum for 1 h and 24 h, and the reaction mix were then analyzed using PAGE-Coomassie stain to assess IgG cleavage (FIG. 1A). The results showed more IgG cleavage by media and cell lysates of IdeS^{op1} and IdeS^{op2} cells at 1 h incubation, compared to those of IdeS cells. Similar results with lower IgG cleavage were also seen by the media from IdeS cells than of IdeS^{op1} and IdeS^{op2} cells at 24 h incubation (FIG. 1A). These data indicate the codon-optimization of IdeS gene may enhance the expression and/or the function of the recombinant protein. Further, the cell lysates were processed to purify the IdeS. Purified IdeS samples (normalized) were incubated with human serum for 1 h and were then analyzed by PAGE-Coomassie stain (FIG. 1B). The results showed more effective IgG cleavage by IdeS^{op2}. Therefore, the IdeS^{op2} construct (also called Ab-C) was designated as the product for further development.

Rabbitized MPS IIIA mouse model with pre-existing αAAV9-Abs IdeS effectively cleaves IgGs from a broad range of species, including humans, primates, sheep, and rabbits.

However, it does not cleave mouse IgG. For the generation of a MPS IIIA mouse model with preexisting αAAV9-Abs, rabbits were immunized with a rAAV9 viral vector (5×10¹² vg/kg) via an IV injection to obtain high levels of αAAV9-Abs. Blood samples were collected at 8 wk pi and αAAV9-IgD was detected in the serum at 1:1,024,000. Serum samples were then processed to purify rabbit IgG (Rb-IgG) using Msgne™Protein A Beads (Promega). The purified Rb IgG was then formulated in PBS in the volume correlated with IgG concentration in serum and assayed for with αAAV9-IgG by binding ELISA. The results showed that the αAAV9-IgG titer of the purified Rb-IgG was 1:512,000. To generate the mouse model with αAAV9-Abs, the purified Rb-IgG was injected into MPS IIIA mice by an IV injection via tail vein.

Rapid and effective IgG cleavage by purified Ab-C To assess the effects of Ab-C in cleavage of rabbit IgG, purified Ab-C at different concentrations was incubated with 10 μl of purified rabbit IgG and then analyzed by PAGE/Coomassie stain. The results showed rapid, effective and dose-responsive IgG cleavage by the Ab-C (FIG. 2), supporting the potential of Ab-C in depletion of preexisting αAAV-Abs for viral vector-mediated gene delivery.

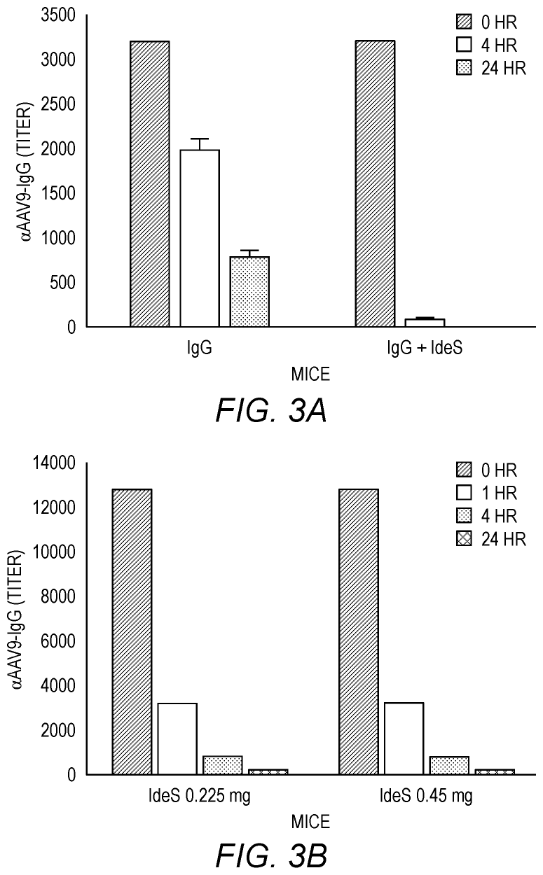

Efficient depletion of αAAV9-positive IgG for systemic rAAV9-hSGSH gene delivery in mice First, to assess the function of Ab-C in vivo in rabbitized mouse model, 6-8 wk-old wt mice were treated with different amounts of purified Rb-IgG in 100 μl (diluted in PBS) by an IV injection via tail vein. At 10 minutes post IgG infusion, subsets of the animals were given an IV injection of Ab-C at different doses (n=2-3/group). Blood samples were collected for analyses at 0 h, 1 h, 4 h, and/or 24h post Ab-C injection. Serum samples were assayed by ELISA for αAAV9-IgG to determine the changes in αAAV9-Abs and assess the impact of Ab-C on Rb-IgG depletion. Similar to in vitro data, the IgG depletion by Ab-C is rapid and efficient in mice (FIGS. 3A-3B) and there appeared to be a threshold of Ab-C dose for optimal IgG depletion (FIG. 3B), though the actual threshold is unclear. A significant decrease was also observed in αAAV9-IgG in mice without Ab-C treatment (FIG. 3A), possibly due to innate immune clearance and natural decay, though the mechanism is unclear.

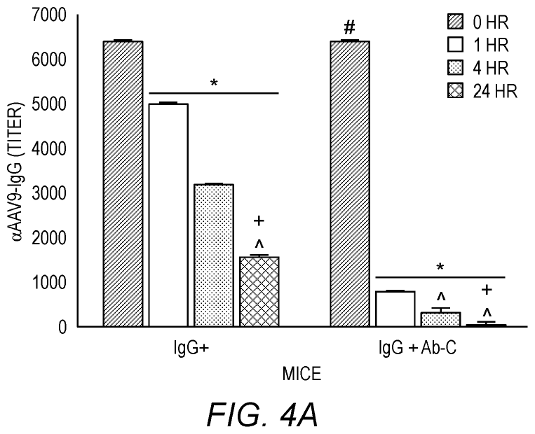

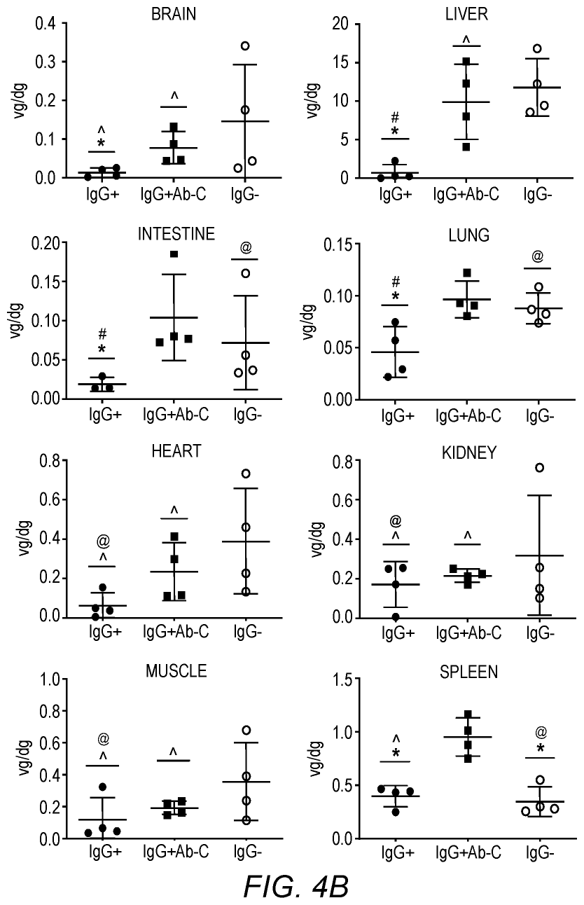

Further, experiments were performed in MPS IIIA mice (6-8 wk-old) to assess the potential of Ab-C-facilitated IgG depletion for systemic AAV-hSGSH gene delivery. Three groups of 14 MPS IIIA mice (n=4-5/group) were used: 1) mice given IV Rb-IgG only, 2) mice given IgG followed by IV Ab-C (0.25 mg/kg), and 3) non-treated mice. All mice were treated with an IV injection of 1×10¹³ vg/kg scAAV9-hSGSH vector, at 24 h after Ab-C injection. Serum samples were assayed by ELISA for αAAV9-IgG at 0 h, 1 h, 4 h, and 24 h post Ab-C treatment (FIG. 4A). At 1 wk post vector injection, necropsy was performed and tissues were assayed by qPCR for vector genome (vg) copy numbers (FIG. 4B). A rapid and effective depletion of αAAV9-IgG from 1:6,400 to <1:100 was observed 24 h post Ab-C infusion, while a much slower decrease of αAAV9-IgG to 1:1,600-1:2,000 was seen in mice without Ab-C treatment (FIG. 4A). Notably, no detectable αAAV9-IgG was observed in mice given the vector only. Importantly, qPCR detected significantly low vg copies in all tested tissues in mice received Rb-IgG without Ab-C treatment, while no significant differences in tissue vg copies were observed in mice given both the Rb-IgG and Ab-C, compared to mice negative for Rb-IgG (FIG. 4B). These data demonstrate that the newly developed Ab-C is functional and effective in depleting the target IgG, and retain the transduction efficiency of systemically delivered AAV9 vectors. This transient Ab-C approach will make all MPS IIIA patients eligible to receive scAAV9-hSGSH gene replacement therapy, including patients with preexisting αAAV9-Abs, as well as the potential for re-administration of the vector.

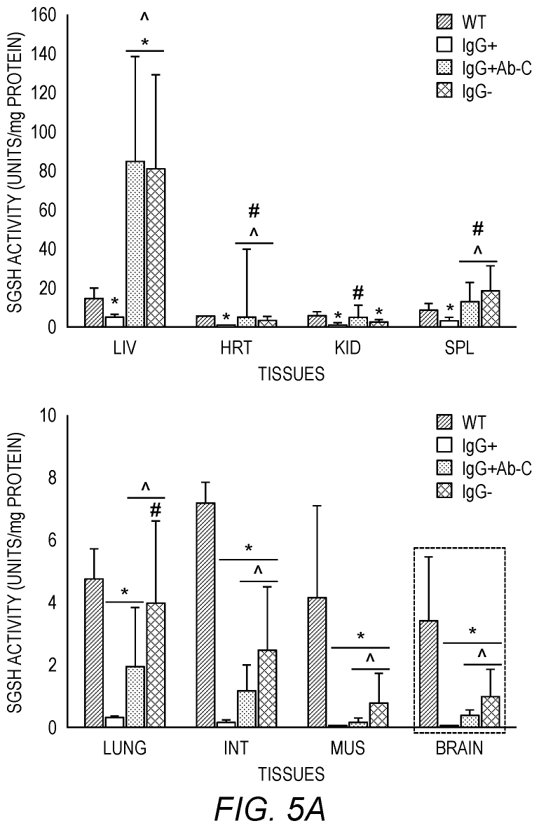

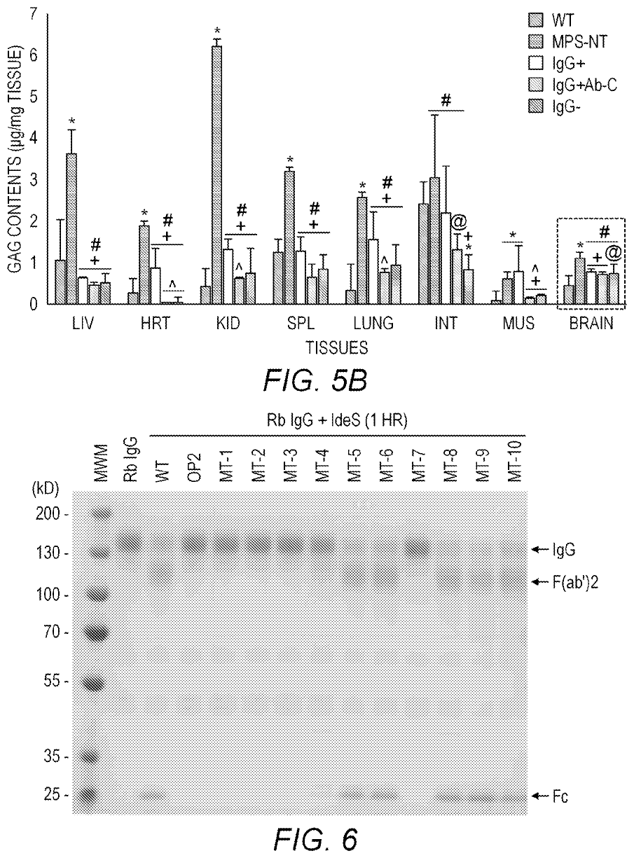

Rapid expression of functional rSGSH in the CNS and peripheral tissues. Further, the impacts of Ab-C pre-treatment on IV AAV9 gene delivery were assessed. Tissues from the experimental mice and controls (n=4) were assayed for SGSH activity at 1 wk post vector infusion. SGSH activity was detected at above WT levels in the liver, at WT or close to WT levels in the spleen and heart, at below WT levels in the brain, lung, intestine, kidney, and skeletal muscles, in αAAV9-Ab⁺ MPS IIIA mice treated with Ab-C and in αAAV9-IgG⁻ MPS IIIA control mice (FIG. 5A), with no significant differences in tissue SGSH activity levels between these 2 cohorts (FIG. 5A). However, significantly lower SGSH activity was detected in all tested tissues in αAAV9-IgG⁺ MPS IIIA mice without Ab-C treatment (FIG. 5A). These data further indicate that the IV Ab-C infusion effectively depleted the pre-existing αAAV9-Abs and enabled the rapid expression of enzymatically functional rSGSH in MPS IIIA mice.

Rapid clearance of tissue GAG storage in the CNS and peripherv. Further, the functionality of the AAV9-mediated rSGSH at 1 wk post vector treatment was assessed. Tissues (n=4/group) were assayed for GAG content. As shown in FIG. 5B, the vector treatments resulted in significant reduction of GAG content in the majority of tested tissues in all 3 cohorts. The GAG contents were detected at normal or below normal levels in the liver and spleen in all 3 cohorts, and close to normal levels in the heart, kidney, lung and muscle in αAAV9-Ab⁺ MPS IIIA mice treated with Ab-C and αAAV9-Ab⁻ MPS IIIA mice (FIG. 5B). The results also showed reduced but not normalized brain GAG levels in all 3 vector treated cohorts (FIG. 5B). While statistically insignificant, higher GAG contents were detected in the heart, kidney, lung and muscle in αAAV9-Ab⁺ MPS IIIA mice without Ab-C treatment, than in Ab-C-treated αAAV9-Ab⁺ MPS IIIA mice or αAAV9-Ab⁻ MPS IIIA mice (FIG. 5B). These data further demonstrate that the AAV9-mediated rSGSH is functional, leading to the rapid clearance of GAG storage in MPS IIIA mice (FIG. 5B), even in tissues with relatively low vg copies (FIG. 4B) and SGSH activity (FIG. 5A).

Further modification of Ab-C protein. Further, the stability and proteolytic function of our Ab-C product was improved. The IdeS protein amino acid (aa) sequences were modified by prediction of mutations in silico, using SWISS-MODEL bioinformatics. A homology model of the IdeS enzyme from Streptococcus pyogenes was generated through the SWISS-MODEL server using PDB ID: 1Y08 as a template. The Rosetta software package was employed to identify mutations to the enzyme according to two distinct strategies. The first focused on identifying disulfide bonds throughout the protein. The disulfidizer mover within RosettaScripts considered all pairs of residues for suitability of forming a disulfide bond strictly from geometric constraints. Models were then scored according to a Relax protocol within RosettaScripts that repacks side chains and minimizes the energy of the model. Those cysteine substitutions with a dslf_13 score term <−0.3 were considered for experimental validation. The second strategy considered combinations of mutations in three mutation zones on the enzyme. Zone 1 and 2 allowed all amino acids to be mutated within 20 Å of residue 274 and 119, respectively. Zone 3 allowed all mutations across the entire protein. To limit the number of mutations emerging from the simulations, simulations providing various bonuses to the native amino acid score were also completed (0, 1, 2, and 5 native residue bonus). The final models were ranked by score and visually inspected to identify preferred sequences. Shown in Table 2, 10 IdeS mutants were generated.

TABLE 2
Table 1 IdeS mutants generated
with SWISS-MODEL bioinformatics

| Mutant | Number of | | SEQ |
|---|---|---|---|
| # | aa mutations | Zone | ID NO. |
| 1 | 144 | 3 | 12 |
| 2 | 83 | 3 | 15 |
| 3 | 39 | 3 | 18 |
| 4 | 2 | 3 | 21 |
| 5 | 14 | 1 | 24 |
| 6 | 5 | 1 | 27 |
| 7 | 9 | 2 | 30 |
| 8 | 2 | — | 33 |
| 9 | 2 | — | 36 |
| 10 | 2 | — | 39 |

The expression and functionality of the new IdeS mutant proteins were assessed. The plasmid-containing BL21 cells were grown and processed to obtain cell lysates. The normalized cell lysates were incubated at 37° C. with 10 μl purified rabbit IgG for 1 h and 24 h, and the reaction mixes were then analyzed using PAGE-CBC (coomassie blue) staining to assess IgG cleavage (FIG. 6). The results showed rapid IgG degradation, with more IgG cleavage by cell lysates from cells transduced with constructs of mutant 6, 8, or 9 at 1 h incubation, compared to that from cells transduced with WT IdeS plasmid, with the most effective IgG cleavage shown by mutant 9 followed by mutants 6 and 8, (FIG. 6). Mutant 5 and 10 appeared to cleave IgG at similar rates to WT IdeS, while mutant 4 yielded weak cleavage and mutants 1-3 and 7 did not yield functional IgG-degrading protein products (FIG. 6). These data demonstrate that IdeS protein products from mutants 6, 8 and 9 are functional and codon-optimization of the IdeS gene may either enhance the expression of IdeS or enhance the specific proteolytic activity of the recombinant protein in bacteria. Therefore, mutants 5, 6, 8, 9, and 10 have the potential to be further developed as the IdeS product.

In summary, to address the challenge of preexisting αAAV-Abs in the translation of AAV-mediated gene therapy, a new effective Ab-depleting product was developed, IdeS by codon-optimization. The IdeS product, as well as some mutant IdeS proteins, were shown to rapidly degrade human and rabbit IgG and demonstrated great potential in clearance of pre-existing αAAV9-Abs for systemic delivery of scAAV9-hSGSH vector in a MPS IIIA mouse model. These studies support further development of IdeS and mutant IdeS for its application in the translation of rAAV gene therapy in future clinical application in all patients in need, including original administration and the potential re-administration in MPS and other lysosomal storage diseases and ultimate commercialization.

### REFERENCES

1. Nathwani A C, Tuddenham E G, Rangarajan S, et al. Adenovirus-associated virus vector-mediated gene transfer in hemophilia B. The New England journal of medicine. 2011; 365(25):2357-2365.
2. Manno C S, Pierce G F, Arruda V R, et al. Successful transduction of liver in hemophilia by AAV-Factor IX and limitations imposed by the host immune response. Nature medicine. 2006; 12(3):342-347.
3. Nathwani A C, Rosales C, McIntosh J, et al. Long-term safety and efficacy following systemic administration of a self-complementary AAV vector encoding human FIX pseudotyped with serotype 5 and 8 capsid proteins. Mol Ther. 2011; 19(5):876-885.
4. Foust K D, Wang X, McGovern V L, et al. Rescue of the spinal muscular atrophy phenotype in a mouse model by early postnatal delivery of SMN. Nature biotechnology. 2010; 28(3):271-274.
5. Fu H, Dirosario J, Killedar S, Zaraspe K, McCarty D M. Correction of neurological disease of mucopolysaccharidosis IIIB in adult mice by rAAV9 trans-blood-brain barrier gene delivery. Mol Ther. 2011; 19(6):1025-1033.
6. Ruzo A, Marco S, Garcia M, et al. Correction of Pathological Accumulation of Glycosaminoglycans in Central Nervous System and Peripheral Tissues of MPSIIIA Mice Through Systemic AAV9 Gene Transfer. Hum Gene Ther. 2012.
7. Mendell J R, Al-Zaidy S, Shell R, et al. Single-Dose Gene-Replacement Therapy for Spinal Muscular Atrophy. N Engl J Med. 2017; 377(18):1713-1722.
8. Foust K D, Nurre E, Montgomery C L, Hernandez A, Chan C M, Kaspar B K. Intravascular AAV9 preferentially targets neonatal neurons and adult astrocytes. Nature biotechnology. 2009; 27(1):59-65.
9. Duque S, Joussemet B, Riviere C, et al. Intravenous administration of self-complementary AAV9 enables transgene delivery to adult motor neurons. Mol Ther. 2009; 17(7):1187-1196.
10. Boutin S, Monteilhet V, Veron P, et al. Prevalence of serum IgG and neutralizing factors against adeno-associated virus (AAV) types 1, 2, 5, 6, 8, and 9 in the healthy population: implications for gene therapy using AAV vectors. Hum Gene Ther. 2010; 21(6):704-712.
11. Calcedo R, Vandenberghe L H, Gao G, Lin J, Wilson J M. Worldwide epidemiology of neutralizing antibodies to adeno-associated viruses. J Infect Dis. 2009; 199(3):381-390.
12. Fu H, Meadows A S, Pineda R J, et al. Differential Prevalence of Antibodies Against Adeno-Associated Virus in Healthy Children and Patients with Mucopolysaccharidosis III: Perspective for AAV-Mediated Gene Therapy. Human gene therapy Clinical development. 2017; 28(4):187-196.
13. Scallan C D, Jiang H, Liu T, et al. Human immunoglobulin inhibits liver transduction by AAV vectors at low AAV2 neutralizing titers in SCID mice. Blood. 2006; 107(5):1810-1817.
14. Wang L, Calcedo R, Bell P, et al. Impact of pre-existing immunity on gene transfer to nonhuman primate liver with adeno-associated virus 8 vectors. Hum Gene Ther. 2011; 22(11):1389-1401.
15. Mingozzi F, Anguela X M, Pavani G, et al. Overcoming preexisting humoral immunity to AAV using capsid decoys. Science translational medicine. 2013; 5(194):194ra192.
16. Maersch S, Huber A, Buning H, Hallek M, Perabo L. Optimization of stealth adeno-associated virus vectors by randomization of immunogenic epitopes. Virology. 2010; 397(1):167-175.
17. Asokan A, Samulski R J. AAV does the shuffle. Nature biotechnology. 2006; 24(2):158-160.
18. Lee G K, Maheshri N, Kaspar B, Schaffer D V. PEG conjugation moderately protects adeno-associated viral vectors against antibody neutralization. Biotechnology and bioengineering. 2005; 92(1):24-34.
19. McIntosh J H, Cochrane M, Cobbold S, et al. Successful attenuation of humoral immunity to viral capsid and transgenic protein following AAV-mediated gene transfer with a non-depleting CD4 antibody and cyclosporine. Gene therapy. 2012; 19(1):78-85.
20. Mingozzi F, Chen Y, Murphy S L, et al. Pharmacological modulation of humoral immunity in a nonhuman primate model of AAV gene transfer for hemophilia B. Mol Ther. 2012; 20(7):1410-1416.
21. Corti M, Elder M, Falk D, et al. B-Cell Depletion is Protective Against Anti-AAV Capsid Immune Response: A Human Subject Case Study. Molecular therapy Methods & clinical development. 2014; 1.
22. Chicoine L G, Montgomery C L, Bremer W G, et al. Plasmapheresis eliminates the negative impact of AAV antibodies on microdystrophin gene expression following vascular delivery. Mol Ther. 2014; 22(2):338-347.
23. Chicoine L G, Rodino-Klapac L R, Shao G, et al. Vascular delivery of rAAVrh74.MCK.GALGT2 to the gastroenemius muscle of the rhesus macaque stimulates the expression of dystrophin and laminin alpha2 surrogates. Mol Ther. 2014; 22(4):713-724.
24. Velazquez V M, Meadows A S, Pineda R J, Camboni M, McCarty D M, Fu H. Effective Depletion of Pre-existing Anti-AAV Antibodies Requires Broad Immune Targeting. Molecular therapy Methods & clinical development. 2017; 4:159-168.
25. Monteilhet V, Saheb S, Boutin S, et al. A 10 patient case report on the impact of plasmapheresis upon neutralizing factors against adeno-associated virus (AAV) types 1, 2, 6, and 8. Mol Ther. 2011; 19(11):2084-2091.
26. von Pawel-Rammingen U, Johansson B P, Bjorck L. IdeS, a novel streptococcal cysteine proteinase with unique specificity for immunoglobulin G. The EMBO journal. 2002; 21(7):1607-1615.
27. von Pawel-Rammingen U, Johansson B P, Tapper H, Bjorck L. Streptococcus pyogenes and phagocytic killing. Nature medicine. 2002; 8(10):1044-1045; author reply 1045-1046.
28. Vincents B, von Pawel-Rammingen U, Bjorck L, Abrahamson M. Enzymatic characterization of the streptococcal endopeptidase, IdeS, reveals that it is a cysteine protease with strict specificity for IgG cleavage due to exosite binding. Biochemistry. 2004; 43(49):15540-15549.
29. Wenig K, Chatwell L, von Pawel-Rammingen U, Bjorck L, Huber R, Sondermann P. Structure of the streptococcal endopeptidase IdeS, a cysteine proteinase with strict specificity for IgG. Proceedings of the National Academy of Sciences of the United States of America. 2004; 101(50):17371-17376.
30. Johansson B P, Shannon O, Bjorck L. IdeS: a bacterial proteolytic enzyme with therapeutic potential. PLoS One. 2008; 3(2):e1692.
31. Ryan M H, Petrone D, Nemeth J F, Bamathan E, Bjorck L, Jordan R E. Proteolysis of purified IgGs by human and bacterial enzymes in vitro and the detection of specific proteolytic fragments of endogenous IgG in rheumatoid synovial fluid. Molecular immunology. 2008; 45(7):1837-1846.
32. Yang R, Otten M A, Hellmark T, et al. Successful treatment of experimental glomerulonephritis with IdeS and EndoS, IgG-degrading streptococcal enzymes. Nephrology, dialysis, transplantation: official publication of the European Dialysis and Transplant Association—European Renal Association. 2010; 25(8):2479-2486.
33. Winstedt L, Jamum S, Nordahl E A, et al. Complete Removal of Extracellular IgG Antibodies in a Randomized Dose-Escalation Phase I Study with the Bacterial Enzyme IdeS—A Novel Therapeutic Opportunity. PloS one. 2015; 10(7):e0132011.
34. Lorant T, Bengtsson M, Eich T, et al. Safety, immunogenicity, pharmacokinetics, and efficacy of degradation of anti-HLA antibodies by IdeS (Imlifidase) in chronic kidney disease patients. Am J Transplant. 2018.
35. Lonze B E, Tatapudi V S, Weldon E P, et al. IdeS (Imlifidase): A Novel Agent That Cleaves Human IgG and Permits Successful Kidney Transplantation Across High-strength Donor-specific Antibody. Ann Surg. 2018; 268(3):488-496.
36. Leborgne C, Barbon E, Alexander J M, et al. IgG-cleaving endopeptidase enables in vivo gene therapy in the presence of anti-AAV neutralizing antibodies. Nature medicine. 2020; 26(7):1096-1101.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

IdeS^{op2} sequence (1,023 bp)
(SEQ ID NO: 1)
cgtaagcggtgctatagtacaagtgcggtcgtactggcggctgttaccttgttcgcgctcagtgtggatcgtggtgtaatcgcggata
gcttttcggcgaaccaggagattcggtattccgaagtcacaccatatcatgtaacgtctgtatggacgaaaggtgtaacgcctccg
gccaaattcacacagggcgaggacgtattccacgccccatatgtcgccaaccaaggttggtatgatattacgaagacgtttaacg
gcaaggacgacctgttgtgcggggcggccaccgcagggaacatgttgcattggtggtttgaccaaaacaaagaaaaaatcga
agcctatctgaaaaagcacccagataagcagaaaattatgttcggcgatcaagagctcctcgatgtccggaaagtgattaacac
gaagggggatcagacgaatagtgagctgtttaactattttcgcgacaaagctttcccggggttatccgcccgtcgcattggcgtcat
gcctgacctcgtacttgacatgttcattaatgggtattatctgaatgtatacaaaacccagacaactgatgtaaatcgcacatatcag
gagaaagatcggcggggtggcatttttgatgccgtatttacacgtggtgaccagtccaaattattaactagccgtcatgatttcaag
gagaagaacttaaaggaaatctctgatttaatcaaaaaggaattgactgaaggtaaggctttaggccttagccatacttatgcaa
atgtgcgtattaaccatgttattaacttgtggggggcagactttgactcgaacggcaatttaaaagccatctatgtcacggacagcg
acagcaatgcttcgattggtatgaaaaagtattttgtaggcgtaaattcagcgggtaaagttgctatcagtgccaaagagatcaag
gaggacaatatcggcgctcaggttcttggtttattcacgctgagcacaggccaagattcttggaatcaaacaaactaa
IdeS^{op1} sequence (1,023 bp)
(SEQ ID NO: 2)
cgtaagcgctgttactcgaccagtgcggttgtactggctgcggtgaccctttttgccctctcggtggaccgtggggtcattgcagattc
tttctccgcgaatcaagagattcggtattctgaggtgactccttaccacgtaacatcggtatggactaaaggcgttacgccgcctgct
aaattcactcagggtgaggatgtattccatgccccttatgtggctaaccagggttggtacgacatcacgaaaaccttcaatggcaa
agacgacctcttatgtggcgcagcgactgcggggaacatgttgcactggtggttcgatcaaaataaagaaaaaattgaggcata
tctgaaaaagcaccctgacaagcaaaagattatgttcggcgaccaggaactgcttgacgtgcgtaaggtgatcaacactaaag
gggaccaaaccaactccgaactgtttaattatttccgcgacaaagctttccctggtttatcggcacggcggattggcgttatgccgg
atctggtgttggacatgtttattaatggctattaccttaacgtatacaaaacacaaactacagatgtaaatcgcacatatcaagaga
aggaccgtcgtgggggcattttcgatgccgtattcacacgtggggatcaaagtaagcttctgacctcgcggcatgatttcaaagaa
aaaaatctcaaagagatcagcgacttaattaagaaggagcttacggagggtaaagcactggggctcagtcatacatacgcca
acgtccgcattaatcatgtaattaatctgtggggcgctgacttcgattcgaatggcaatttgaaggcgatctatgtgaccgatagtga
cagtaacgcttcgattggcatgaagaaatattttgtcggcgtcaactccgctggcaaagtcgcgatttctgccaaagagatcaagg
aggataatattggggctcaagtgttgggtttgttcacattatcaacaggccaagactcttggaaccagacaaattaa
Plasmid pGEX-IdeS^{op2} (6,004 bp)
(SEQ ID NO: 3)
acgttatcgactgcacggtgcaccaatgcttctggcgtcaggcagccatcggaagctgtggtatggctgtgcaggtcgtaaatcac
tgcataattcgtgtcgctcaaggcgcactcccgttctggataatgttttttgcgccgacatcataacggttctggcaaatattctgaaat
gagctgttgacaattaatcatcggctcgtataatgtgtggaattgtgagcggataacaatttcacacaggaaacagtattcatgtcc
cctatactaggttattggaaaattaagggccttgtgcaacccactcgacttcttttggaatatcttgaagaaaaatatgaagagcattt
gtatgagcgcgatgaaggtgataaatggcgaaacaaaaagtttgaattgggtttggagtttcccaatcttccttattatattgatggtg
atgttaaattaacacagtctatggccatcatacgttatatagctgacaagcacaacatgttgggtggttgtccaaaagagcgtgca
gagatttcaatgcttgaaggagcggttttggatattagatacggtgtttcgagaattgcatatagtaaagactttgaaactctcaaagt
tgattttcttagcaagctacctgaaatgctgaaaatgttcgaagatcgtttatgtcataaaacatatttaaatggtgatcatgtaaccca
tcctgacttcatgttgtatgacgctcttgatgttgttttatacatggacccaatgtgcctggatgcgttcccaaaattagtttgttttaaaaa
acgtattgaagctatcccacaaattgataagtacttgaaatccagcaagtatatagcatggcctttgcagggctggcaagccacgt
ttggtggtggcgaccatcctccaaaatcggatctggaagttctgttccaggggcccctgggatcccgtaagcggtgctatagtaca
agtgcggtcgtactggcggctgttaccttgttcgcgctcagtgtggatcgtggtgtaatcgcggatagcttttcggcgaaccaggag
attcggtattccgaagtcacaccatatcatgtaacgtctgtatggacgaaaggtgtaacgcctccggccaaattcacacagggcg
aggacgtattccacgccccatatgtcgccaaccaaggttggtatgatattacgaagacgtttaacggcaaggacgacctgttgtg
cggggcggccaccgcagggaacatgttgcattggtggtttgaccaaaacaaagaaaaaatcgaagcctatctgaaaaagcac
ccagataagcagaaaattatgttcggcgatcaagagctcctcgatgtccggaaagtgattaacacgaagggggatcagacga
atagtgagctgtttaactattttcgcgacaaagctttcccggggttatccgcccgtcgcattggcgtcatgcctgacctcgtacttgac
atgttcattaatgggtattatctgaatgtatacaaaacccagacaactgatgtaaatcgcacatatcaggagaaagatcggggg
gtggcatttttgatgccgtatttacacgtggtgaccagtccaaattattaactagccgtcatgatttcaaggagaagaacttaaagga
aatctctgatttaatcaaaaaggaattgactgaaggtaaggctttaggccttagccatacttatgcaaatgtgcgtattaaccatgtta
ttaacttgtggggggcagactttgactcgaacggcaatttaaaagccatctatgtcacggacagcgacagcaatgcttcgattggt
atgaaaaagtattttgtaggcgtaaattcagcgggtaaagttgctatcagtgccaaagagatcaaggaggacaatatcggcgctc
aggttcttggtttattcacgctgagcacaggccaagattcttggaatcaaacaaactaagaattcccgggtcgactcgagcggcc
gcatcgtgactgactgacgatctgcctcgcgcgtttcggtgatgacggtgaaaacctctgacacatgcagctcccggagacggtc
acagcttgtctgtaagcggatgccgggagcagacaagcccgtcagggcgcgtcagcgggtgttggcgggtgtcggggcgcag
ccatgacccagtcacgtagcgatagcggagtgtataattcttgaagacgaaagggcctcgtgatacgcctatttttataggttaatgt
catgataataatggtttcttagacgtcaggtggcacttttcggggaaatgtgcgcggaacccctatttgtttatttttctaaatacattca
aatatgtatccgctcatgagacaataaccctgataaatgcttcaataatattgaaaaaggaagagtatgagtattcaacatttccgt
gtcgcccttattcccttttttgcggcattttgccttcctgtttttgctcacccagaaacgctggtgaaagtaaaagatgctgaagatcagtt
gggtgcacgagtgggttacatcgaactggatctcaacagcggtaagatccttgagagttttcgccccgaagaacgttttccaatga
tgagcacttttaaagttctgctatgtggcgcggtattatcccgtgttgacgccgggcaagagcaactcggtcgccgcatacactattc
tcagaatgacttggttgagtactcaccagtcacagaaaagcatcttacggatggcatgacagtaagagaattatgcagtgctgcc
ataaccatgagtgataacactgcggccaacttacttctgacaacgatcggaggaccgaaggagctaaccgcttttttgcacaaca
tgggggatcatgtaactcgccttgatcgttgggaaccggagctgaatgaagccataccaaacgacgagcgtgacaccacgatg
cctgcagcaatggcaacaacgttgcgcaaactattaactggcgaactacttactctagcttcccggcaacaattaatagactggat
ggaggcggataaagttgcaggaccacttctgcgctcggcccttccggctggctggtttattgctgataaatctggagccggtgagc
gtgggtctcgcggtatcattgcagcactggggccagatggtaagccctcccgtatcgtagttatctacacgacggggagtcaggc
aactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaac
gtgagttttcgttccactgagcgtcagaccccgtagaaaagatcaaaggatcttcttgagatcctttttttctgcgcgtaatctgctgctt
gcaaacaaaaaaaccaccgctaccagcggtggtttgtttgccggatcaagagctaccaactctttttccgaaggtaactggcttca
gcagagcgcagataccaaatactgtccttctagtgtagccgtagttaggccaccacttcaagaactctgtagcaccgcctacata
cctcgctctgctaatcctgttaccagtggctgctgccagtggcgataagtcgtgtcttaccgggttggactcaagacgatagttaccg
gataaggcgcagcggtcgggctgaacggggggttcgtgcacacagcccagcttggagcgaacgacctacaccgaactgaga
tacctacagcgtgagctatgagaaagcgccacgcttcccgaagggagaaaggcggacaggtatccggtaagcggcagggtc
ggaacaggagagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttcctggcctttt
gctggccttttgctcacatgttctttcctgcgttatcccctgattctgtggataaccgtattaccgcctttgagtgagctgataccgctcgc
cgcagccgaacgaccgagcgcagcgagtcagtgagcgaggaagcggaagagcgcctgatgcggtattttctccttacgcatct
gtgcggtatttcacaccgcataaattccgacaccatcgaatggtgcaaaacctttcgcggtatggcatgatagcgcccggaagag
agtcaattcagggtggtgaatgtgaaaccagtaacgttatacgatgtcgcagagtatgccggtgtctcttatcagaccgtttcccgc
gtggtgaaccaggccagccacgtttctgcgaaaacgcgggaaaaagtggaagcggcgatggcggagctgaattacattccca
accgcgtggcacaacaactggcgggcaaacagtcgttgctgattggcgttgccacctccagtctggccctgcacgcgccgtcgc
aaattgtcgcggcgattaaatctcgcgccgatcaactgggtgccagcgtggtggtgtcgatggtagaacgaagcggcgtcgaag
cctgtaaagcggcggtgcacaatcttctcgcgcaacgcgtcagtgggctgatcattaactatccgctggatgaccaggatgccatt
gctgtggaagctgcctgcactaatgttccggcgttatttcttgatgtctctgaccagacacccatcaacagtattattttctcccatgaa
gacggtacgcgactgggcgtggagcatctggtcgcattgggtcaccagcaaatcgcgctgttagcgggcccattaagttctgtctc
ggcgcgtctgcgtctggctggctggcataaatatctcactcgcaatcaaattcagccgatagcggaacgggaaggcgactgga
gtgccatgtccggttttcaacaaaccatgcaaatgctgaatgagggcatcgttcccactgcgatgctggttgccaacgatcagatg
gcgctgggcgcaatgcgcgccattaccgagtccgggctgcgcgttggtgcggatatctcggtagtgggatacgacgataccgaa
gacagctcatgttatatcccgccgtcaaccaccatcaaacaggattttcgcctgctggggcaaaccagcgtggaccgcttgctgc
aactctctcagggccaggcggtgaagggcaatcagctgttgcccgtctcactggtgaaaagaaaaaccaccctggcgcccaat
acgcaaaccgcctctccccgcgcgttggccgattcattaatgcagctggcacgacaggtttcccgactggaaagcgggcagtga
gcgcaacgcaattaatgtgagttagctcactcattaggcaccccaggctttacactttatgcttccggctcgtatgttgtgtggaattgt
gagcggataacaatttcacacaggaaacagctatgaccatgattacggattcactggccgtcgttttacaacgtcgtgactggga
aaaccctggcgttacccaacttaatcgccttgcagcacatccccctttcgccagctggcgtaatagcgaagaggcccgcaccga
tcgcccttcccaacagttgcgcagcctgaatggcgaatggcgctttgcctggtttccggcaccagaagcggtgccggaaagctg
gctggagtgcgatcttcctgaggccgatactgtcgtcgtcccctcaaactggcagatgcacggttacgatgcgcccatctacacca
acgtaacctatcccattacggtcaatccgccgtttgttcccacggagaatccgacgggttgttactcgctcacatttaatgttgatgaa
agctggctacaggaaggccagacgcgaattatttttgatggcgttggaatt
Plasmid pGEX-IdeS^{op1} (6,004 bp)
(SEQ ID NO: 4)
acgttatcgactgcacggtgcaccaatgcttctggcgtcaggcagccatcggaagctgtggtatggctgtgcaggtcgtaaatcac
tgcataattcgtgtcgctcaaggcgcactcccgttctggataatgttttttgcgccgacatcataacggttctggcaaatattctgaaat
gagctgttgacaattaatcatcggctcgtataatgtgtggaattgtgagcggataacaatttcacacaggaaacagtattcatgtcc
cctatactaggttattggaaaattaagggccttgtgcaacccactcgacttcttttggaatatcttgaagaaaaatatgaagagcattt
gtatgagcgcgatgaaggtgataaatggcgaaacaaaaagtttgaattgggtttggagtttcccaatcttccttattatattgatggtg
atgttaaattaacacagtctatggccatcatacgttatatagctgacaagcacaacatgttgggtggttgtccaaaagagcgtgca
gagatttcaatgcttgaaggagcggttttggatattagatacggtgtttcgagaattgcatatagtaaagactttgaaactctcaaagt
tgattttcttagcaagctacctgaaatgctgaaaatgttcgaagatcgtttatgtcataaaacatatttaaatggtgatcatgtaaccca
tcctgacttcatgttgtatgacgctcttgatgttgttttatacatggacccaatgtgcctggatgcgttcccaaaattagtttgttttaaaaa
acgtattgaagctatcccacaaattgataagtacttgaaatccagcaagtatatagcatggcctttgcagggctggcaagccacgt
ttggtggtggcgaccatcctccaaaatcggatctggaagttctgttccaggggcccctgggatcccgtaagcgctgttactcgacc
agtgcggttgtactggctgcggtgaccctttttgccctctcggtggaccgtggggtcattgcagattctttctccgcgaatcaagagatt
cggtattctgaggtgactccttaccacgtaacatcggtatggactaaaggcgttacgccgcctgctaaattcactcagggtgagga
tgtattccatgccccttatgtggctaaccagggttggtacgacatcacgaaaaccttcaatggcaaagacgacctcttatgtggcgc
agcgactgcggggaacatgttgcactggtggttcgatcaaaataaagaaaaaattgaggcatatctgaaaaagcaccctgaca
agcaaaagattatgttcggcgaccaggaactgcttgacgtgcgtaaggtgatcaacactaaaggggaccaaaccaactccga
actgtttaattatttccgcgacaaagctttccctggtttatcggcacggcggattggcgttatgccggatctggtgttggacatgtttatta
atggctattaccttaacgtatacaaaacacaaactacagatgtaaatcgcacatatcaagagaaggaccgtcgtgggggcatttt
cgatgccgtattcacacgtggggatcaaagtaagcttctgacctcgcggcatgatttcaaagaaaaaaatctcaaagagatcag
cgacttaattaagaaggagcttacggagggtaaagcactggggctcagtcatacatacgccaacgtccgcattaatcatgtaatt
aatctgtggggcgctgacttcgattcgaatggcaatttgaaggcgatctatgtgaccgatagtgacagtaacgcttcgattggcatg
aagaaatattttgtcggcgtcaactccgctggcaaagtcgcgatttctgccaaagagatcaaggaggataatattggggctcaag
tgttgggtttgttcacattatcaacaggccaagactcttggaaccagacaaattaagaattcccgggtcgactcgagcggccgcat
cgtgactgactgacgatctgcctcgcgcgtttcggtgatgacggtgaaaacctctgacacatgcagctcccggagacggtcaca
gcttgtctgtaagcggatgccgggagcagacaagcccgtcagggcgcgtcagcgggtgttggcgggtgtcggggcgcagcca
tgacccagtcacgtagcgatagcggagtgtataattcttgaagacgaaagggcctcgtgatacgcctatttttataggttaatgtcat
gataataatggtttcttagacgtcaggtggcacttttcggggaaatgtgcgcggaacccctatttgtttatttttctaaatacattcaaat
atgtatccgctcatgagacaataaccctgataaatgcttcaataatattgaaaaaggaagagtatgagtattcaacatttccgtgtc
gcccttattcccttttttgcggcattttgccttcctgtttttgctcacccagaaacgctggtgaaagtaaaagatgctgaagatcagttgg
gtgcacgagtgggttacatcgaactggatctcaacagcggtaagatccttgagagttttcgccccgaagaacgttttccaatgatg
agcacttttaaagttctgctatgtggcgcggtattatcccgtgttgacgccgggcaagagcaactcggtcgccgcatacactattctc
agaatgacttggttgagtactcaccagtcacagaaaagcatcttacggatggcatgacagtaagagaattatgcagtgctgccat
aaccatgagtgataacactgcggccaacttacttctgacaacgatcggaggaccgaaggagctaaccgcttttttgcacaacatg
ggggatcatgtaactcgccttgatcgttgggaaccggagctgaatgaagccataccaaacgacgagcgtgacaccacgatgcc
tgcagcaatggcaacaacgttgcgcaaactattaactggcgaactacttactctagcttcccggcaacaattaatagactggatg
gaggcggataaagttgcaggaccacttctgcgctcggcccttccggctggctggtttattgctgataaatctggagccggtgagcg
tgggtctcgcggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaagtttactcata
tatactttagattgatttaaaacttcatttttaatttaaaaggatctaggtgaagatcctttttgataatctcatgaccaaaatcccttaacg
tgagttttcgttccactgagcgtcagaccccgtagaaaagatcaaaggatcttcttgagatcctttttttctgcgcgtaatctgctgcttg
caaacaaaaaaaccaccgctaccagcggtggtttgtttgccggatcaagagctaccaactctttttccgaaggtaactggcttcag
cagagcgcagataccaaatactgtccttctagtgtagccgtagttaggccaccacttcaagaactctgtagcaccgcctacatacc
tcgctctgctaatcctgttaccagtggctgctgccagtggcgataagtcgtgtcttaccgggttggactcaagacgatagttaccgga
taaggcgcagcggtcgggctgaacggggggttcgtgcacacagcccagcttggagcgaacgacctacaccgaactgagata
cctacagcgtgagctatgagaaagcgccacgcttcccgaagggagaaaggcggacaggtatccggtaagcggcagggtcg
gaacaggagagcgcacgagggagcttccagggggaaacgcctggtatctttatagtcctgtcgggtttcgccacctctgacttga
gcgtcgatttttgtgatgctcgtcaggggggcggagcctatggaaaaacgccagcaacgcggcctttttacggttcctggccttttgc
tggccttttgctcacatgttctttcctgcgttatcccctgattctgtggataaccgtattaccgcctttgagtgagctgataccgctcgccg
cagccgaacgaccgagcgcagcgagtcagtgagcgaggaagcggaagagcgcctgatgcggtattttctccttacgcatctgt
gcggtatttcacaccgcataaattccgacaccatcgaatggtgcaaaacctttcgcggtatggcatgatagcgcccggaagaga
gtcaattcagggtggtgaatgtgaaaccagtaacgttatacgatgtcgcagagtatgccggtgtctcttatcagaccgtttcccgcgt
ggtgaaccaggccagccacgtttctgcgaaaacgcgggaaaaagtggaagcggcgatggcggagctgaattacattcccaa
ccgcgtggcacaacaactggcgggcaaacagtcgttgctgattggcgttgccacctccagtctggccctgcacgcgccgtcgca
aattgtcgcggcgattaaatctcgcgccgatcaactgggtgccagcgtggtggtgtcgatggtagaacgaagcggcgtcgaagc
ctgtaaagcggcggtgcacaatcttctcgcgcaacgcgtcagtgggctgatcattaactatccgctggatgaccaggatgccattg
ctgtggaagctgcctgcactaatgttccggcgttatttcttgatgtctctgaccagacacccatcaacagtattattttctcccatgaag
acggtacgcgactgggcgtggagcatctggtcgcattgggtcaccagcaaatcgcgctgttagcgggcccattaagttctgtctcg
gcgcgtctgcgtctggctggctggcataaatatctcactcgcaatcaaattcagccgatagcggaacgggaaggcgactggagt
gccatgtccggttttcaacaaaccatgcaaatgctgaatgagggcatcgttcccactgcgatgctggttgccaacgatcagatggc
gctgggcgcaatgcgcgccattaccgagtccgggctgcgcgttggtgcggatatctcggtagtgggatacgacgataccgaag
acagctcatgttatatcccgccgtcaaccaccatcaaacaggattttcgcctgctggggcaaaccagcgtggaccgcttgctgca
actctctcagggccaggcggtgaagggcaatcagctgttgcccgtctcactggtgaaaagaaaaaccaccctggcgcccaata
cgcaaaccgcctctccccgcgcgttggccgattcattaatgcagctggcacgacaggtttcccgactggaaagcgggcagtgag
cgcaacgcaattaatgtgagttagctcactcattaggcaccccaggctttacactttatgcttccggctcgtatgttgtgtggaattgtg
agcggataacaatttcacacaggaaacagctatgaccatgattacggattcactggccgtcgttttacaacgtcgtgactgggaa
aaccctggcgttacccaacttaatcgccttgcagcacatccccctttcgccagctggcgtaatagcgaagaggcccgcaccgatc
gcccttcccaacagttgcgcagcctgaatggcgaatggcgctttgcctggtttccggcaccagaagcggtgccggaaagctggc
tggagtgcgatcttcctgaggccgatactgtcgtcgtcccctcaaactggcagatgcacggttacgatgcgcccatctacaccaac
gtaacctatcccattacggtcaatccgccgtttgttcccacggagaatccgacgggttgttactcgctcacatttaatgttgatgaaag
ctggctacaggaaggccagacgcgaattatttttgatggcgttggaatt
IdeS^{ori} sequence (1,023 bp)
(SEQ ID NO: 5)
agaaaaagatgctattcaacttcagctgtagtattggcagcagtgactttatttgctctatcggtagatcgtggtgttatagcagatagt
ttttctgctaatcaagagattagatattcggaagtaacaccttatcatgttacttccgtttggaccaaaggagttactcctccagcaaa
attcactcaaggcgaagatgtttttcacgctccttatgttgctaaccaaggatggtatgatattaccaaaacattcaatggaaaagac
gatcttctttgcggggctgccacagcagggaatatgcttcactggtggttcgatcaaaacaaagaaaaaattgaagcatatctaa
aaaaacacccagataaacaaaaaatcatgtttggtgatcaagaattattggatgtaagaaaagttattaataccaaaggtgacc
aaacaaatagcgagctttttaattatttccgagataaagctttccccggtttgtcagcacgccgaattggagttatgcctgatcttgtttt
agatatgtttatcaatggttattacttaaatgtttataagacacagactactgatgtcaatagaacctatcaagagaaagatcgccga
ggtggtatttttgacgccgtatttacaagaggtgatcaaagtaagctattgacaagtcgtcatgattttaaagaaaaaaatctcaaa
gaaatcagtgatctcattaagaaagagttaaccgaaggcaaggctctaggcctatcacacacctacgctaacgtacgcatcaac
catgttataaacctgtggggagctgactttgattctaacgggaaccttaaagctatttatgtaacagactctgatagtaatgcatctatt
ggtatgaagaaatactttgttggtgttaattccgctggaaaagtagctatttctgctaaagaaataaaagaagataatattggtgctc
aagtactagggttatttacactttcaacagggcaagatagttggaatcagaccaattaa
Plasmid pGEX-IdeS^{ori} (6,004 bp)
(SEQ ID NO: 6)
acgttatcgactgcacggtgcaccaatgcttctggcgtcaggcagccatcggaagctgtggtatggctgtgcaggtcgtaaatcac
tgcataattcgtgtcgctcaaggcgcactcccgttctggataatgttttttgcgccgacatcataacggttctggcaaatattctgaaat
gagctgttgacaattaatcatcggctcgtataatgtgtggaattgtgagcggataacaatttcacacaggaaacagtattcatgtcc
cctatactaggttattggaaaattaagggccttgtgcaacccactcgacttcttttggaatatcttgaagaaaaatatgaagagcattt
gtatgagcgcgatgaaggtgataaatggcgaaacaaaaagtttgaattgggtttggagtttcccaatcttccttattatattgatggtg
atgttaaattaacacagtctatggccatcatacgttatatagctgacaagcacaacatgttgggtggttgtccaaaagagcgtgca
gagatttcaatgcttgaaggagcggttttggatattagatacggtgtttcgagaattgcatatagtaaagactttgaaactctcaaagt
tgattttcttagcaagctacctgaaatgctgaaaatgttcgaagatcgtttatgtcataaaacatatttaaatggtgatcatgtaaccca
tcctgacttcatgttgtatgacgctcttgatgttgttttatacatggacccaatgtgcctggatgcgttcccaaaattagtttgttttaaaa
aacgtattgaagctatcccacaaattgataagtacttgaaatccagcaagtatatagcatggcctttgcagggctggcaagccacgt
ttggtggtggcgaccatcctccaaaatcggatctggaagttctgttccaggggcccctgggatccagaaaaagatgctattcaact
tcagctgtagtattggcagcagtgactttatttgctctatcggtagatcgtggtgttatagcagatagtttttctgctaatcaagagattag
atattcggaagtaacaccttatcatgttacttccgtttggaccaaaggagttactcctccagcaaaattcactcaaggcgaagatgtt
tttcacgctccttatgttgctaaccaaggatggtatgatattaccaaaacattcaatggaaaagacgatcttctttgcggggctgcca
cagcagggaatatgcttcactggtggttcgatcaaaacaaagaaaaaattgaagcatatctaaaaaaacacccagataaaca
aaaaatcatgtttggtgatcaagaattattggatgtaagaaaagttattaataccaaaggtgaccaaacaaatagcgagctttttaa
ttatttccgagataaagctttccccggtttgtcagcacgccgaattggagttatgcctgatcttgttttagatatgtttatcaatggttatt
acttaaatgtttataagacacagactactgatgtcaatagaacctatcaagagaaagatcgccgaggtggtatttttgacgccgtattta
caagaggtgatcaaagtaagctattgacaagtcgtcatgattttaaagaaaaaaatctcaaagaaatcagtgatctcattaagaa
agagttaaccgaaggcaaggctctaggcctatcacacacctacgctaacgtacgcatcaaccatgttataaacctgtggggagc
tgactttgattctaacgggaaccttaaagctatttatgtaacagactctgatagtaatgcatctattggtatgaagaaatactttgttggt
gttaattccgctggaaaagtagctatttctgctaaagaaataaaagaagataatattggtgctcaagtactagggttatttacactttc
aacagggcaagatagttggaatcagaccaattaagaattcccgggtcgactcgagcggccgcatcgtgactgactgacgatct
gcctcgcgcgtttcggtgatgacggtgaaaacctctgacacatgcagctcccggagacggtcacagcttgtctgtaagcggatgc
cgggagcagacaagcccgtcagggcgcgtcagcgggtgttggcgggtgtcggggcgcagccatgacccagtcacgtagcga
tagcggagtgtataattcttgaagacgaaagggcctcgtgatacgcctatttttataggttaatgtcatgataataatggtttcttagac
gtcaggtggcacttttcggggaaatgtgcgcggaacccctatttgtttatttttctaaatacattcaaatatgtatccgctcatgagaca
ataaccctgataaatgcttcaataatattgaaaaaggaagagtatgagtattcaacatttccgtgtcgcccttattcccttttttgcggc
attttgccttcctgtttttgctcacccagaaacgctggtgaaagtaaaagatgctgaagatcagttgggtgcacgagtgggttacatc
gaactggatctcaacagcggtaagatccttgagagttttcgccccgaagaacgttttccaatgatgagcacttttaaagttctgctat
gtggcgcggtattatcccgtgttgacgccgggcaagagcaactcggtcgccgcatacactattctcagaatgacttggttgagtact
caccagtcacagaaaagcatcttacggatggcatgacagtaagagaattatgcagtgctgccataaccatgagtgataacactg
cggccaacttacttctgacaacgatcggaggaccgaaggagctaaccgcttttttgcacaacatgggggatcatgtaactcgcctt
gatcgttgggaaccggagctgaatgaagccataccaaacgacgagcgtgacaccacgatgcctgcagcaatggcaacaacg
ttgcgcaaactattaactggcgaactacttactctagcttcccggcaacaattaatagactggatggaggcggataaagttgcagg
accacttctgcgctcggcccttccggctggctggtttattgctgataaatctggagccggtgagcgtgggtctcgcggtatcattgca
gcactggggccagatggtaagccctcccgtatcgtagttatctacacgacggggagtcaggcaactatggatgaacgaaatag
acagatcgctgagataggtgcctcactgattaagcattggtaactgtcagaccaagtttactcatatatactttagattgatttaaaac
ttcatttttaatttaaaaggatctaggtgaagatcctttttgataatctcatgaccaaaatcccttaacgtgagttttcgttccactgagcg
tcagaccccgtagaaaagatcaaaggatcttcttgagatcctttttttctgcgcgtaatctgctgcttgcaaacaaaaaaaccaccgc
taccagcggtggtttgtttgccggatcaagagctaccaactctttttccgaaggtaactggcttcagcagagcgcagataccaaat
actgtccttctagtgtagccgtagttaggccaccacttcaagaactctgtagcaccgcctacatacctcgctctgctaatcctgttacc
agtggctgctgccagtggcgataagtcgtgtcttaccgggttggactcaagacgatagttaccggataaggcgcagcggtcggg
ctgaacggggggttcgtgcacacagcccagcttggagcgaacgacctacaccgaactgagatacctacagcgtgagctatga
gaaagcgccacgcttcccgaagggagaaaggcggacaggtatccggtaagcggcagggtcggaacaggagagcgcacg
agggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgttc
tttcctgcgttatcccctgattctgtggataaccgtattaccgcctttgagtgagctgataccgctcgccgcagccgaacgaccgagc
gcagcgagtcagtgagcgaggaagcggaagagcgcctgatgcggtattttctccttacgcatctgtgcggtatttcacaccgcat
aaattccgacaccatcgaatggtgcaaaacctttcgcggtatggcatgatagcgcccggaagagagtcaattcagggtggtgaa
tgtgaaaccagtaacgttatacgatgtcgcagagtatgccggtgtctcttatcagaccgtttcccgcgtggtgaaccaggccagcc
acgtttctgcgaaaacgcgggaaaaagtggaagcggcgatggcggagctgaattacattcccaaccgcgtggcacaacaact
ggcgggcaaacagtcgttgctgattggcgttgccacctccagtctggccctgcacgcgccgtcgcaaattgtcgcggcgattaaa
tctcgcgccgatcaactgggtgccagcgtggtggtgtcgatggtagaacgaagcggcgtcgaagcctgtaaagcggcggtgca
caatcttctcgcgcaacgcgtcagtgggctgatcattaactatccgctggatgaccaggatgccattgctgtggaagctgcctgca
ctaatgttccggcgttatttcttgatgtctctgaccagacacccatcaacagtattattttctcccatgaagacggtacgcgactgggc
gtggagcatctggtcgcattgggtcaccagcaaatcgcgctgttagcgggcccattaagttctgtctcggcgcgtctgcgtctggct
ggctggcataaatatctcactcgcaatcaaattcagccgatagcggaacgggaaggcgactggagtgccatgtccggttttcaa
caaaccatgcaaatgctgaatgagggcatcgttcccactgcgatgctggttgccaacgatcagatggcgctgggcgcaatgcgc
gccattaccgagtccgggctgcgcgttggtgcggatatctcggtagtgggatacgacgataccgaagacagctcatgttatatccc
gccgtcaaccaccatcaaacaggattttcgcctgctggggcaaaccagcgtggaccgcttgctgcaactctctcagggccaggc
ggtgaagggcaatcagctgttgcccgtctcactggtgaaaagaaaaaccaccctggcgcccaatacgcaaaccgcctctcccc
gcgcgttggccgattcattaatgcagctggcacgacaggtttcccgactggaaagcgggcagtgagcgcaacgcaattaatgtg
agttagctcactcattaggcaccccaggctttacactttatgcttccggctcgtatgttgtgtggaattgtgagcggataacaatttcac
acaggaaacagctatgaccatgattacggattcactggccgtcgttttacaacgtcgtgactgggaaaaccctggcgttacccaa
cttaatcgccttgcagcacatccccctttcgccagctggcgtaatagcgaagaggcccgcaccgatcgcccttcccaacagttgc
gcagcctgaatggcgaatggcgctttgcctggtttccggcaccagaagcggtgccggaaagctggctggagtgcgatcttcctga
ggccgatactgtcgtcgtcccctcaaactggcagatgcacggttacgatgcgcccatctacaccaacgtaacctatcccattacg
gtcaatccgccgtttgttcccacggagaatccgacgggttgttactcgctcacatttaatgttgatgaaagctggctacaggaaggc
cagacgcgaattatttttgatggcgttggaatt
IdeS^{op1} protein sequence
(SEQ ID NO: 7)
RKRCYSTSAVVLAAVTLFALSVDRGVIADSFSANQEIRYSEVTPYHVTSVWTKGVTPPAKFT
QGEDVFHAPYVANQGWYDITKTFNGKDDLLCGAATAGNMLHWWFDQNKEKIEAYLKKHP
DKQKIMFGDQELLDVRKVINTKGDQTNSELFNYFRDKAFPGLSARRIGVMPDLVLDMFINGY
YLNVYKTQTTDVNRTYQEKDRRGGIFDAVFTRGDQSKLLTSRHDFKEKNLKEISDLIKKELT
EGKALGLSHTYANVRINHVINLWGADFDSNGNLKAIYVTDSDSNASIGMKKYFVGVNSAGK
VAISAKEIKEDNIGAQVLGLFTLSTGQDSWNQTN
IdeS^{op2} protein sequence
(SEQ ID NO: 8)
RKRCYSTSAVVLAAVTLFALSVDRGVIADSFSANQEIRYSEVTPYHVTSVWTKGVTPPAKFT
QGEDVFHAPYVANQGWYDITKTFNGKDDLLCGAATAGNMLHWWFDQNKEKIEAYLKKHP
DKQKIMFGDQELLDVRKVINTKGDQTNSELFNYFRDKAFPGLSARRIGVMPDLVLDMFINGY
YLNVYKTQTTDVNRTYQEKDRRGGIFDAVFTRGDQSKLLTSRHDFKEKNLKEISDLIKKELT
EGKALGLSHTYANVRINHVINLWGADFDSNGNLKAIYVTDSDSNASIGMKKYFVGVNSAGK
VAISAKEIKEDNIGAQVLGLFTLSTGQDSWNQTN
Ides^{ori} protein sequence
(SEQ ID NO: 9)
RKRCYSTSAVVLAAVTLFALSVDRGVIADSFSANQEIRYSEVTPYHVTSVWTKGVTPPAKFT
QGEDVFHAPYVANQGWYDITKTFNGKDDLLCGAATAGNMLHWWFDQNKEKIEAYLKKHP
DKQKIMFGDQELLDVRKVINTKGDQTNSELFNYFRDKAFPGLSARRIGVMPDLVLDMFINGY
YLNVYKTQTTDVNRTYQEKDRRGGIFDAVFTRGDQSKLLTSRHDFKEKNLKEISDLIKKELT
EGKALGLSHTYANVRINHVINLWGADFDSNGNLKAIYVTDSDSNASIGMKKYFVGVNSAGK
VAISAKEIKEDNIGAQVLGLFTLSTGQDSWNQTN
Plasmid pGEX-Ides^{mt1} (6,004 bp)
(SEQ ID NO: 10)
acgttatcgactgcacggtgcaccaatgcttctggcgtcaggcagccatcggaagctgtggtatggctgtgcaggtcgtaaatca
ctgcataattcgtgtcgctcaaggcgcactcccgttctggataatgttttttgcgccgacatcataacggttctggcaaatattctgaa
atgagctgttgacaattaatcatcggctcgtataatgtgtggaattgtgagcggataacaatttcacacaggaaacagtattcatgt
cccctatactaggttattggaaaattaagggccttgtgcaacccactcgacttcttttggaatatcttgaagaaaaatatgaagagc
atttgtatgagcgcgatgaaggtgataaatggcgaaacaaaaagtttgaattgggtttggagtttcccaatcttccttattatattgatg
gtgatgttaaattaacacagtctatggccatcatacgttatatagctgacaagcacaacatgttgggtggttgtccaaaagagcgtg
cagagatttcaatgcttgaaggagcggttttggatattagatacggtgtttcgagaattgcatatagtaaagactttgaaactctcaa
agttgattttcttagcaagctacctgaaatgctgaaaatgttcgaagatcgtttatgtcataaaacatatttaaatggtgatcatgtaac
ccatcctgacttcatgttgtatgacgctcttgatgttgttttatacatggacccaatgtgcctggatgcgttcccaaaattagtttgttttaa
aaaacgtattgaagctatcccacaaattgataagtacttgaaatccagcaagtatatagcatggcctttgcagggctggcaagcc
acgtttggtggtggcgaccatcctccaaaatcggatctggaagttctgttccaggggcccctgggatccaggaaaagatgttattc
aacaagtgctgtagtcctggcggcggtcactctgttcgcactgagcgttgatcgcggtgtgatcgccgattcttttagcgcgaacca
agaaattcgctacagcgaggtgactccgtaccatgtttgggaaatctggttggacggcaccattccgttttactggtggaccgttac
caaagacatgatctacgtgccgtacatcccaaatatgggcatttacttcctgttcaaaacctttgatggcaaagacgacttactgtg
cggtgctgcgaccgcagttatgatgattttttggtggctgatggttaatcgtgactgggtttggtggtatatgaaagtttttccgtttatgat
ctacatctggaaggacaaccaattgttgctggtcttggtgttggtgatgctgacggcttgggatctgctttacccgcacctgtggctttt
cttccgcgacatcgcctttccaggtttgtcggcgcgtcgtatcggcgtgatgccggatttggtactcgctatgtttttcatgggttatctg
ctgaatgtttataagacccagaccaccgatattgaatataaaccgatgccgagagattggcgtggcggtctcttcctgatgattttct
ggcttggcgatccgcgtttttatatggtgtatcgtcatgattttaaagagctgaatctggagcagatttctcgtatcatgctgtactggttg
ttaaaaggtgtggtgctgggtctgagccacacctatgccaacgtgcgcatcaaccacgttattaacgtttgggcaattgtcctggac
aaggacaacctgctgcgttatatgttcgttgttgattccgattccgacccgcgctggggtctgttcatctggttcatcgacatcaacaa
cgcgggtaaggtggcgattagctggaagcagatcgataaggacaacattggcgcacaggtgctcggcctgttcgtgttgctgca
aggtaaggacttatggaatctgacggactaagaattcccgggtcgactcgagcggccgcatcgtgactgactgacgatctgcct
cgcgcgtttcggtgatgacggtgaaaacctctgacacatgcagctcccggagacggtcacagcttgtctgtaagcggatgccgg
gagcagacaagcccgtcagggcgcgtcagcgggtgttggcgggtgtcggggcgcagccatgacccagtcacgtagcgatag
cggagtgtataattcttgaagacgaaagggcctcgtgatacgcctatttttataggttaatgtcatgataataatggtttcttagacgtc
aggtggcacttttcggggaaatgtgcgcggaacccctatttgtttatttttctaaatacattcaaatatgtatccgctcatgagacaata
accctgataaatgcttcaataatattgaaaaaggaagagtatgagtattcaacatttccgtgtcgcccttattcccttttttgcggcattt
tgccttcctgtttttgctcacccagaaacgctggtgaaagtaaaagatgctgaagatcagttgggtgcacgagtgggttacatcga
actggatctcaacagcggtaagatccttgagagttttcgccccgaagaacgttttccaatgatgagcacttttaaagttctgctatgt
ggcgcggtattatcccgtgttgacgccgggcaagagcaactcggtcgccgcatacactattctcagaatgacttggttgagtactc
accagtcacagaaaagcatcttacggatggcatgacagtaagagaattatgcagtgctgccataaccatgagtgataacactg
cggccaacttacttctgacaacgatcggaggaccgaaggagctaaccgcttttttgcacaacatgggggatcatgtaactcgcct
tgatcgttgggaaccggagctgaatgaagccataccaaacgacgagcgtgacaccacgatgcctgcagcaatggcaacaac
gttgcgcaaactattaactggcgaactacttactctagcttcccggcaacaattaatagactggatggaggcggataaagttgca
ggaccacttctgcgctcggcccttccggctggctggtttattgctgataaatctggagccggtgagcgtgggtctcgcggtatcattg
cagcactggggccagatggtaagccctcccgtatcgtagttatctacacgacggggagtcaggcaactatggatgaacgaaat
agacagatcgctgagataggtgcctcactgattaagcattggtaactgtcagaccaagtttactcatatatactttagattgatttaaa
acttcatttttaatttaaaaggatctaggtgaagatcctttttgataatctcatgaccaaaatcccttaacgtgagttttcgttccactgag
cgtcagaccccgtagaaaagatcaaaggatcttcttgagatcctttttttctgcgcgtaatctgctgcttgcaaacaaaaaaaccac
cgctaccagcggtggtttgtttgccggatcaagagctaccaactctttttccgaaggtaactggcttcagcagagcgcagatacca
aatactgtccttctagtgtagccgtagttaggccaccacttcaagaactctgtagcaccgcctacatacctcgctctgctaatcctgtt
accagtggctgctgccagtggcgataagtcgtgtcttaccgggttggactcaagacgatagttaccggataaggcgcagcggtc
gggctgaacggggggttcgtgcacacagcccagcttggagcgaacgacctacaccgaactgagatacctacagcgtgagct
atgagaaagcgccacgcttcccgaagggagaaaggcggacaggtatccggtaagcggcagggtcggaacaggagagcg
cacgagggagcttccagggggaaacgcctggtatctttatagtcctgtcgggtttcgccacctctgacttgagcgtcgatttttgtgat
gctcgtcaggggggcggagcctatggaaaaacgccagcaacgcggcctttttacggttcctggccttttgctggccttttgctcaca
tgttctttcctgcgttatcccctgattctgtggataaccgtattaccgcctttgagtgagctgataccgctcgccgcagccgaacgacc
gagcgcagcgagtcagtgagcgaggaagcggaagagcgcctgatgcggtattttctccttacgcatctgtgcggtatttcacacc
gcataaattccgacaccatcgaatggtgcaaaacctttcgcggtatggcatgatagcgcccggaagagagtcaattcagggtg
gtgaatgtgaaaccagtaacgttatacgatgtcgcagagtatgccggtgtctcttatcagaccgtttcccgcgtggtgaaccaggc
cagccacgtttctgcgaaaacgcgggaaaaagtggaagcggcgatggcggagctgaattacattcccaaccgcgtggcaca
acaactggcgggcaaacagtcgttgctgattggcgttgccacctccagtctggccctgcacgcgccgtcgcaaattgtcgcggc
gattaaatctcgcgccgatcaactgggtgccagcgtggtggtgtcgatggtagaacgaagcggcgtcgaagcctgtaaagcgg
cggtgcacaatcttctcgcgcaacgcgtcagtgggctgatcattaactatccgctggatgaccaggatgccattgctgtggaagct
gcctgcactaatgttccggcgttatttcttgatgtctctgaccagacacccatcaacagtattattttctcccatgaagacggtacgcg
actgggcgtggagcatctggtcgcattgggtcaccagcaaatcgcgctgttagcgggcccattaagttctgtctcggcgcgtctgc
gtctggctggctggcataaatatctcactcgcaatcaaattcagccgatagcggaacgggaaggcgactggagtgccatgtccg
gttttcaacaaaccatgcaaatgctgaatgagggcatcgttcccactgcgatgctggttgccaacgatcagatggcgctgggcgc
aatgcgcgccattaccgagtccgggctgcgcgttggtgcggatatctcggtagtgggatacgacgataccgaagacagctcatg
ttatatcccgccgtcaaccaccatcaaacaggattttcgcctgctggggcaaaccagcgtggaccgcttgctgcaactctctcag
ggccaggcggtgaagggcaatcagctgttgcccgtctcactggtgaaaagaaaaaccaccctggcgcccaatacgcaaacc
gcctctccccgcgcgttggccgattcattaatgcagctggcacgacaggtttcccgactggaaagcgggcagtgagcgcaacg
caattaatgtgagttagctcactcattaggcaccccaggctttacactttatgcttccggctcgtatgttgtgtggaattgtgagcggat
aacaatttcacacaggaaacagctatgaccatgattacggattcactggccgtcgttttacaacgtcgtgactgggaaaaccctg
gcgttacccaacttaatcgccttgcagcacatccccctttcgccagctggcgtaatagcgaagaggcccgcaccgatcgcccttc
ccaacagttgcgcagcctgaatggcgaatggcgctttgcctggtttccggcaccagaagcggtgccggaaagctggctggagt
gcgatcttcctgaggccgatactgtcgtcgtcccctcaaactggcagatgcacggttacgatgcgcccatctacaccaacgtaac
ctatcccattacggtcaatccgccgtttgttcccacggagaatccgacgggttgttactcgctcacatttaatgttgatgaaagctgg
ctacaggaaggccagacgcgaattatttttgatggcgttggaatt
Ides^{mt1} DNA sequence (1,023 bp)
(SEQ ID NO: 11)
aggaaaagatgttattcaacaagtgctgtagtcctggcggcggtcactctgttcgcactgagcgttgatcgcggtgtgatcgccga
ttcttttagcgcgaaccaagaaattcgctacagcgaggtgactccgtaccatgtttgggaaatctggttggacggcaccattccgttt
tactggtggaccgttaccaaagacatgatctacgtgccgtacatcccaaatatgggcatttacttcctgttcaaaacctttgatggca
aagacgacttactgtgcggtgctgcgaccgcagttatgatgattttttggtggctgatggttaatcgtgactgggtttggtggtatatga
aagtttttccgtttatgatctacatctggaaggacaaccaattgttgctggtcttggtgttggtgatgctgacggcttgggatctgctttac
ccgcacctgtggcttttcttccgcgacatcgcctttccaggtttgtcggcgcgtcgtatcggcgtgatgccggatttggtactcgctatg
tttttcatgggttatctgctgaatgtttataagacccagaccaccgatattgaatataaaccgatgccgagagattggcgtggcggtc
tcttcctgatgattttctggcttggcgatccgcgtttttatatggtgtatcgtcatgattttaaagagctgaatctggagcagatttctcgta
tcatgctgtactggttgttaaaaggtgtggtgctgggtctgagccacacctatgccaacgtgcgcatcaaccacgttattaacgtttgg
gcaattgtcctggacaaggacaacctgctgcgttatatgttcgttgttgattccgattccgacccgcgctggggtctgttcatctggttc
atcgacatcaacaacgcgggtaaggtggcgattagctggaagcagatcgataaggacaacattggcgcacaggtgctcggc
ctgttcgtgttgctgcaaggtaaggacttatggaatctgacggactaa
Ides^{mt1} Protein sequence (340 aa + stop codon) (mutations underlined)
(SEQ ID NO: 12)
RKRCYSTSAVVLAAVTLFALSVDRGVIADSFSANQEIRYSEVTPYHVWEIWLDGTIPFYWWT
VTKDMIYVPYIPNMGIYFLFKTFDGKDDLLCGAATAVMMIFWWLMVNRDWVWWYMKVFPF
MIYIWKDNQLLLVLVLVMLTAWDLLYPHLWLFFRDIAFPGLSARRIGVMPDLVLAMFFMGYL
LNVYKTQTTDIEYKPMPRDWRGGLFLMIFWLGDPRFYMVYRHDFKELNLEQISRIMLYWLL
KGVVLGLSHTYANVRINHVINVWAIVLDKDNLLRYMFVVDSDSDPRWGLFIWFIDINNAGKV
AISWKQIDKDNIGAQVLGLFVLLQGKDLWNLTD*
Plasmid pGEX-IdeS^{mt2} (6,004 bp)
(SEQ ID NO: 13)
acgttatcgactgcacggtgcaccaatgcttctggcgtcaggcagccatcggaagctgtggtatggctgtgcaggtcgtaaatca
ctgcataattcgtgtcgctcaaggcgcactcccgttctggataatgttttttgcgccgacatcataacggttctggcaaatattctgaa
atgagctgttgacaattaatcatcggctcgtataatgtgtggaattgtgagcggataacaatttcacacaggaaacagtattcatgt
cccctatactaggttattggaaaattaagggccttgtgcaacccactcgacttcttttggaatatcttgaagaaaaatatgaagagc
atttgtatgagcgcgatgaaggtgataaatggcgaaacaaaaagtttgaattgggtttggagtttcccaatcttccttattatattgatg
gtgatgttaaattaacacagtctatggccatcatacgttatatagctgacaagcacaacatgttgggtggttgtccaaaagagcgtg
cagagatttcaatgcttgaaggagcggttttggatattagatacggtgtttcgagaattgcatatagtaaagactttgaaactctcaa
agttgattttcttagcaagctacctgaaatgctgaaaatgttcgaagatcgtttatgtcataaaacatatttaaatggtgatcatgtaac
ccatcctgacttcatgttgtatgacgctcttgatgttgttttatacatggacccaatgtgcctggatgcgttcccaaaattagtttgttttaa
aaaacgtattgaagctatcccacaaattgataagtacttgaaatccagcaagtatatagcatggcctttgcagggctggcaagcc
acgtttggtggtggcgaccatcctccaaaatcggatctggaagttctgttccaggggcccctgggatccaggaaaagatgttattc
aacaagtgctgtagttctggctgcggttaccctgttcgccctgagcgttgaccgcggtgtgattgccgactccttcagcgccaatca
agaaatccgttacagcgaagtgactccgtatcacgttatgaccgtttgggtcaaagacgtgatcccgccacaatggtggaccca
aactaaggactacattcacgcgccgttttacccgaaccagggctggtatgcgctcttcaaaacgtttaatggtaaggacgacctg
ctttgcggtgcggcgaccgcaatcgtgatgatttggtggtggctggatgtcaacaaagagaaaatcgaagagtacttgaagaaa
catccgcaccgtcagcaaatttggaaagatgatcagctgttgctggacatctgggctgtgattaataccaagtgggatatgacga
acccgcacctgtttatctacttccgcgacattgcgtttccgggtctgtcggctcgtcgcattggtgttatgccggatttggttctgatgat
gttcataatgggctatccgctgaatgtttataaaacccagacgaccgatgtggactacaagccgatgccacaagattggcgtggt
ggtatttttatcgacgtgttctggctgggcgacccgcgttactacttagttagccgtcatgattttaaagagaaaaacttgaaggagat
ctccgatgtcatcctgaagttattgttggaaggaaagatgctgggtctgtcccatacctatgcgaatgttagaatcaaccacgtgat
caacctgtggggtgcggtgttcgattctaacggcttgctgaaggcaatctatgtgaccgattctgatagcgaccctcgtgatggtat
gctcttatatttcgtcaacgtaaacagcgctggcaaggtggcaattagcaccaagccgattgacaaagataatatcggcgcgca
ggttctgggcctgttcaccctcgacgtgggcaaagacctgtggaaccagaccaactaagaattcccgggtcgactcgagcggc
cgcatcgtgactgactgacgatctgcctcgcgcgtttcggtgatgacggtgaaaacctctgacacatgcagctcccggagacggt
cacagcttgtctgtaagcggatgccgggagcagacaagcccgtcagggcgcgtcagcgggtgttggcgggtgtcggggcgca
gccatgacccagtcacgtagcgatagcggagtgtataattcttgaagacgaaagggcctcgtgatacgcctatttttataggttaat
gtcatgataataatggtttcttagacgtcaggtggcacttttcggggaaatgtgcgcggaacccctatttgtttatttttctaaatacattc
aaatatgtatccgctcatgagacaataaccctgataaatgcttcaataatattgaaaaaggaagagtatgagtattcaacatttcc
gtgtcgcccttattcccttttttgcggcattttgccttcctgtttttgctcacccagaaacgctggtgaaagtaaaagatgctgaagatca
gttgggtgcacgagtgggttacatcgaactggatctcaacagcggtaagatccttgagagttttcgccccgaagaacgttttccaa
tgatgagcacttttaaagttctgctatgtggcgcggtattatcccgtgttgacgccgggcaagagcaactcggtcgccgcatacact
attctcagaatgacttggttgagtactcaccagtcacagaaaagcatcttacggatggcatgacagtaagagaattatgcagtgct
gccataaccatgagtgataacactgcggccaacttacttctgacaacgatcggaggaccgaaggagctaaccgcttttttgcac
aacatgggggatcatgtaactcgccttgatcgttgggaaccggagctgaatgaagccataccaaacgacgagcgtgacacca
cgatgcctgcagcaatggcaacaacgttgcgcaaactattaactggcgaactacttactctagcttcccggcaacaattaataga
ctggatggaggcggataaagttgcaggaccacttctgcgctcggcccttccggctggctggtttattgctgataaatctggagccg
gtgagcgtgggtctcgcggtatcattgcagcactggggccagatggtaagccctcccgtatcgtagttatctacacgacggggag
tcaggcaactatggatgaacgaaatagacagatcgctgagataggtgcctcactgattaagcattggtaactgtcagaccaagtt
tactcatatatactttagattgatttaaaacttcatttttaatttaaaaggatctaggtgaagatcctttttgataatctcatgaccaaaatc
ccttaacgtgagttttcgttccactgagcgtcagaccccgtagaaaagatcaaaggatcttcttgagatcctttttttctgcgcgtaatc
tgctgcttgcaaacaaaaaaaccaccgctaccagcggtggtttgtttgccggatcaagagctaccaactctttttccgaaggtaac
tggcttcagcagagcgcagataccaaatactgtccttctagtgtagccgtagttaggccaccacttcaagaactctgtagcaccgc
ctacatacctcgctctgctaatcctgttaccagtggctgctgccagtggcgataagtcgtgtcttaccgggttggactcaagacgat
agttaccggataaggcgcagcggtcgggctgaacggggggttcgtgcacacagcccagcttggagcgaacgacctacaccg
aactgagatacctacagcgtgagctatgagaaagcgccacgcttcccgaagggagaaaggcggacaggtatccggtaagc
ggcagggtcggaacaggagagcgcacgagggagcttccagggggaaacgcctggtatctttatagtcctgtcgggtttcgcca
cctctgacttgagcgtcgatttttgtgatgctcgtcaggggggcggagcctatggaaaaacgccagcaacgcggcctttttacggtt
cctggccttttgctggccttttgctcacatgttctttcctgcgttatcccctgattctgtggataaccgtattaccgcctttgagtgagctga
taccgctcgccgcagccgaacgaccgagcgcagcgagtcagtgagcgaggaagcggaagagcgcctgatgcggtattttctc
cttacgcatctgtgcggtatttcacaccgcataaattccgacaccatcgaatggtgcaaaacctttcgcggtatggcatgatagcg
cccggaagagagtcaattcagggtggtgaatgtgaaaccagtaacgttatacgatgtcgcagagtatgccggtgtctcttatcag
accgtttcccgcgtggtgaaccaggccagccacgtttctgcgaaaacgcgggaaaaagtggaagcggcgatggcggagctg
aattacattcccaaccgcgtggcacaacaactggcgggcaaacagtcgttgctgattggcgttgccacctccagtctggccctgc
acgcgccgtcgcaaattgtcgcggcgattaaatctcgcgccgatcaactgggtgccagcgtggtggtgtcgatggtagaacgaa
gcggcgtcgaagcctgtaaagcggcggtgcacaatcttctcgcgcaacgcgtcagtgggctgatcattaactatccgctggatg
accaggatgccattgctgtggaagctgcctgcactaatgttccggcgttatttcttgatgtctctgaccagacacccatcaacagtat
tattttctcccatgaagacggtacgcgactgggcgtggagcatctggtcgcattgggtcaccagcaaatcgcgctgttagcgggc
ccattaagttctgtctcggcgcgtctgcgtctggctggctggcataaatatctcactcgcaatcaaattcagccgatagcggaacg
ggaaggcgactggagtgccatgtccggttttcaacaaaccatgcaaatgctgaatgagggcatcgttcccactgcgatgctggtt
gccaacgatcagatggcgctgggcgcaatgcgcgccattaccgagtccgggctgcgcgttggtgcggatatctcggtagtggg
atacgacgataccgaagacagctcatgttatatcccgccgtcaaccaccatcaaacaggattttcgcctgctggggcaaaccag
cgtggaccgcttgctgcaactctctcagggccaggcggtgaagggcaatcagctgttgcccgtctcactggtgaaaagaaaaa
ccaccctggcgcccaatacgcaaaccgcctctccccgcgcgttggccgattcattaatgcagctggcacgacaggtttcccgac
tggaaagcgggcagtgagcgcaacgcaattaatgtgagttagctcactcattaggcaccccaggctttacactttatgcttccggc
tcgtatgttgtgtggaattgtgagcggataacaatttcacacaggaaacagctatgaccatgattacggattcactggccgtcgtttt
acaacgtcgtgactgggaaaaccctggcgttacccaacttaatcgccttgcagcacatccccctttcgccagctggcgtaatagc
gaagaggcccgcaccgatcgcccttcccaacagttgcgcagcctgaatggcgaatggcgctttgcctggtttccggcaccaga
agcggtgccggaaagctggctggagtgcgatcttcctgaggccgatactgtcgtcgtcccctcaaactggcagatgcacggtta
cgatgcgcccatctacaccaacgtaacctatcccattacggtcaatccgccgtttgttcccacggagaatccgacgggttgttactc
gctcacatttaatgttgatgaaagctggctacaggaaggccagacgcgaattatttttgatggcgttggaatt
IdeS^{mt2} DNA sequence (1,023 bp)
(SEQ ID NO: 14)
aggaaaagatgttattcaacaagtgctgtagttctggctgcggttaccctgttcgccctgagcgttgaccgcggtgtgattgccgact
ccttcagcgccaatcaagaaatccgttacagcgaagtgactccgtatcacgttatgaccgtttgggtcaaagacgtgatcccgcc
acaatggtggacccaaactaaggactacattcacgcgccgttttacccgaaccagggctggtatgcgctcttcaaaacgtttaat
ggtaaggacgacctgctttgcggtgcggcgaccgcaatcgtgatgatttggtggtggctggatgtcaacaaagagaaaatcgaa
gagtacttgaagaaacatccgcaccgtcagcaaatttggaaagatgatcagctgttgctggacatctgggctgtgattaatacca
agtgggatatgacgaacccgcacctgtttatctacttccgcgacattgcgtttccgggtctgtcggctcgtcgcattggtgttatgccg
gatttggttctgatgatgttcataatgggctatccgctgaatgtttataaaacccagacgaccgatgtggactacaagccgatgcca
caagattggcgtggtggtatttttatcgacgtgttctggctgggcgacccgcgttactacttagttagccgtcatgattttaaagagaa
aaacttgaaggagatctccgatgtcatcctgaagttattgttggaaggaaagatgctgggtctgtcccatacctatgcgaatgttag
aatcaaccacgtgatcaacctgtggggtgcggtgttcgattctaacggcttgctgaaggcaatctatgtgaccgattctgatagcg
accctcgtgatggtatgctcttatatttcgtcaacgtaaacagcgctggcaaggtggcaattagcaccaagccgattgacaaagat
aatatcggcgcgcaggttctgggcctgttcaccctcgacgtgggcaaagacctgtggaaccagaccaactaa
IdeS^{mt2} Protein sequence (340 aa + stop codon) (mutations underlined)
(SEQ ID NO: 15)
RKRCYSTSAVVLAAVTLFALSVDRGVIADSFSANQEIRYSEVTPYHVMTVWVKDVIPPQWW
TQTKDYIHAPFYPNQGWYALFKTFNGKDDLLCGAATAIVMIWWWLDVNKEKIEEYLKKHPH
RQQIWKDDQLLLDIWAVINTKWDMTNPHLFIYFRDIAFPGLSARRIGVMPDLVLMMFIMGYP
LNVYKTQTTDVDYKPMPQDWRGGIFIDVFWLGDPRYYLVSRHDFKEKNLKEISDVILKLLLE
GKMLGLSHTYANVRINHVINLWGAVFDSNGLLKAIYVTDSDSDPRDGMLLYFVNVNSAGKV
AISTKPIDKDNIGAQVLGLFTLDVGKDLWNQTN*
Plasmid pGEX-IdeS-mt3 (6,004 bp)
(SEQ ID NO: 16)
acgttatcgactgcacggtgcaccaatgcttctggcgtcaggcagccatcggaagctgtggtatggctgtgcaggtcgtaaatca
ctgcataattcgtgtcgctcaaggcgcactcccgttctggataatgttttttgcgccgacatcataacggttctggcaaatattctgaa
atgagctgttgacaattaatcatcggctcgtataatgtgtggaattgtgagcggataacaatttcacacaggaaacagtattcatgt
cccctatactaggttattggaaaattaagggccttgtgcaacccactcgacttcttttggaatatcttgaagaaaaatatgaagagc
atttgtatgagcgcgatgaaggtgataaatggcgaaacaaaaagtttgaattgggtttggagtttcccaatcttccttattatattgatg
gtgatgttaaattaacacagtctatggccatcatacgttatatagctgacaagcacaacatgttgggtggttgtccaaaagagcgtg
cagagatttcaatgcttgaaggagcggttttggatattagatacggtgtttcgagaattgcatatagtaaagactttgaaactctcaa
agttgattttcttagcaagctacctgaaatgctgaaaatgttcgaagatcgtttatgtcataaaacatatttaaatggtgatcatgtaac
ccatcctgacttcatgttgtatgacgctcttgatgttgttttatacatggacccaatgtgcctggatgcgttcccaaaattagtttgttttaa
aaaacgtattgaagctatcccacaaattgataagtacttgaaatccagcaagtatatagcatggcctttgcagggctggcaagcc
acgtttggtggtggcgaccatcctccaaaatcggatctggaagttctgttccaggggcccctgggatccaggaaaagatgttattc
aacaagtgctgtagttttggcggcggttacgctcttcgccctgtcggtagatagaggcgttattgcagactccttcagcgcgaacca
ggagatccgctacagcgaagttacgccgtaccacgtgacttccgtgtgggttaaaggcgttacgccgcctcagtggtggaccca
aaccgaagacttcttttacgccccatacgtcccgaatcaaggttggtatgatttgaccaagacctttaacggcaaggatgatctgct
ttgcggtgctgcgaccgcgatcaacatgctgtggtggtggtttgacgtgaacaaagagaagattgaggagtatctcaagaagca
tccggacaaacaaaaaatcatgaaagacgaccaagagctgctagacgtgcgcaaggttatcaataccaaatgggatcaaac
gaatagccacctgttcctgtattttcgtgatattgcgtttccgggtctgtctgcgcgtcgtatcggagtaatgccggatttggttctgatca
tgttcatcatgggttattacctgaatgtttacaaaacgcagaccacggacgtcaaccgtaccccgcaagaaaaggactggcgtg
gtggcatttttatcgctgtcttcaccttgggtgatccgagcaaatatctgaccagccgtcatgattttaaagaaaagaacctgaagg
agattagcgacaccatcaaaaaactgttggaggaaggcaaaatgctgggcctgagccacacctatgctaacgtgcgcattaac
cacgtgattaatctgtggggtgcagatttcgactctaatggcctgctcaaggccatctacgtgactgacagcgactctaacccgtc
gatcggcatgttgaagtacttcgtgggcgtgaacagcgcgggtaaggtggcgatttccctgaaacagattgatgaagataacatc
ggtgcacaggttctgggcttgttcaccctggacaccggtcaggatctgtggaatcagaccaactaagaattcccgggtcgactcg
agcggccgcatcgtgactgactgacgatctgcctcgcgcgtttcggtgatgacggtgaaaacctctgacacatgcagctcccgg
agacggtcacagcttgtctgtaagcggatgccgggagcagacaagcccgtcagggcgcgtcagcgggtgttggcgggtgtcg
gggcgcagccatgacccagtcacgtagcgatagcggagtgtataattcttgaagacgaaagggcctcgtgatacgcctattttta
taggttaatgtcatgataataatggtttcttagacgtcaggtggcacttttcggggaaatgtgcgcggaacccctatttgtttatttttcta
aatacattcaaatatgtatccgctcatgagacaataaccctgataaatgcttcaataatattgaaaaaggaagagtatgagtattc
aacatttccgtgtcgcccttattcccttttttgcggcattttgccttcctgtttttgctcacccagaaacgctggtgaaagtaaaagatgct
gaagatcagttgggtgcacgagtgggttacatcgaactggatctcaacagcggtaagatccttgagagttttcgccccgaagaa
cgttttccaatgatgagcacttttaaagttctgctatgtggcgcggtattatcccgtgttgacgccgggcaagagcaactcggtcgcc
gcatacactattctcagaatgacttggttgagtactcaccagtcacagaaaagcatcttacggatggcatgacagtaagagaatt
atgcagtgctgccataaccatgagtgataacactgcggccaacttacttctgacaacgatcggaggaccgaaggagctaaccg
cttttttgcacaacatgggggatcatgtaactcgccttgatcgttgggaaccggagctgaatgaagccataccaaacgacgagcg
tgacaccacgatgcctgcagcaatggcaacaacgttgcgcaaactattaactggcgaactacttactctagcttcccggcaaca
attaatagactggatggaggcggataaagttgcaggaccacttctgcgctcggcccttccggctggctggtttattgctgataaatc
tggagccggtgagcgtgggtctcgcggtatcattgcagcactggggccagatggtaagccctcccgtatcgtagttatctacacg
acggggagtcaggcaactatggatgaacgaaatagacagatcgctgagataggtgcctcactgattaagcattggtaactgtca
gaccaagtttactcatatatactttagattgatttaaaacttcatttttaatttaaaaggatctaggtgaagatcctttttgataatctcatg
accaaaatcccttaacgtgagttttcgttccactgagcgtcagaccccgtagaaaagatcaaaggatcttcttgagatcctttttttct
gcgcgtaatctgctgcttgcaaacaaaaaaaccaccgctaccagcggtggtttgtttgccggatcaagagctaccaactctttttcc
gaaggtaactggcttcagcagagcgcagataccaaatactgtccttctagtgtagccgtagttaggccaccacttcaagaactct
gtagcaccgcctacatacctcgctctgctaatcctgttaccagtggctgctgccagtggcgataagtcgtgtcttaccgggttggact
caagacgatagttaccggataaggcgcagcggtcgggctgaacggggggttcgtgcacacagcccagcttggagcgaacga
cctacaccgaactgagatacctacagcgtgagctatgagaaagcgccacgcttcccgaagggagaaaggcggacaggtatc
cggtaagcggcagggtcggaacaggagagcgcacgagggagcttccagggggaaacgcctggtatctttatagtcctgtcgg
gtttcgccacctctgacttgagcgtcgatttttgtgatgctcgtcaggggggcggagcctatggaaaaacgccagcaacgcggcc
tttttacggttcctggccttttgctggccttttgctcacatgttctttcctgcgttatcccctgattctgtggataaccgtattaccgccttt
gagtgagctgataccgctcgccgcagccgaacgaccgagcgcagcgagtcagtgagcgaggaagcggaagagcgcctgatgc
ggtattttctccttacgcatctgtgcggtatttcacaccgcataaattccgacaccatcgaatggtgcaaaacctttcgcggtatggc
atgatagcgcccggaagagagtcaattcagggtggtgaatgtgaaaccagtaacgttatacgatgtcgcagagtatgccggtgt
ctcttatcagaccgtttcccgcgtggtgaaccaggccagccacgtttctgcgaaaacgcgggaaaaagtggaagcggcgatgg
cggagctgaattacattcccaaccgcgtggcacaacaactggcgggcaaacagtcgttgctgattggcgttgccacctccagtct
ggccctgcacgcgccgtcgcaaattgtcgcggcgattaaatctcgcgccgatcaactgggtgccagcgtggtggtgtcgatggt
agaacgaagcggcgtcgaagcctgtaaagcggcggtgcacaatcttctcgcgcaacgcgtcagtgggctgatcattaactatc
cgctggatgaccaggatgccattgctgtggaagctgcctgcactaatgttccggcgttatttcttgatgtctctgaccagacacccat
caacagtattattttctcccatgaagacggtacgcgactgggcgtggagcatctggtcgcattgggtcaccagcaaatcgcgctgt
tagcgggcccattaagttctgtctcggcgcgtctgcgtctggctggctggcataaatatctcactcgcaatcaaattcagccgatag
cggaacgggaaggcgactggagtgccatgtccggttttcaacaaaccatgcaaatgctgaatgagggcatcgttcccactgcg
atgctggttgccaacgatcagatggcgctgggcgcaatgcgcgccattaccgagtccgggctgcgcgttggtgcggatatctcg
gtagtgggatacgacgataccgaagacagctcatgttatatcccgccgtcaaccaccatcaaacaggattttcgcctgctgggg
caaaccagcgtggaccgcttgctgcaactctctcagggccaggcggtgaagggcaatcagctgttgcccgtctcactggtgaaa
agaaaaaccaccctggcgcccaatacgcaaaccgcctctccccgcgcgttggccgattcattaatgcagctggcacgacaggt
ttcccgactggaaagcgggcagtgagcgcaacgcaattaatgtgagttagctcactcattaggcaccccaggctttacactttatg
cttccggctcgtatgttgtgtggaattgtgagcggataacaatttcacacaggaaacagctatgaccatgattacggattcactggc
cgtcgttttacaacgtcgtgactgggaaaaccctggcgttacccaacttaatcgccttgcagcacatccccctttcgccagctggcg
taatagcgaagaggcccgcaccgatcgcccttcccaacagttgcgcagcctgaatggcgaatggcgctttgcctggtttccggc
accagaagcggtgccggaaagctggctggagtgcgatcttcctgaggccgatactgtcgtcgtcccctcaaactggcagatgc
acggttacgatgcgcccatctacaccaacgtaacctatcccattacggtcaatccgccgtttgttcccacggagaatccgacggg
ttgttactcgctcacatttaatgttgatgaaagctggctacaggaaggccagacgcgaattatttttgatggcgttggaatt
IdeS^{mt3} DNA sequence (1,023 bp)
(SEQ ID NO: 17)
aggaaaagatgttattcaacaagtgctgtagttttggcggcggttacgctcttcgccctgtcggtagatagaggcgttattgcagact
ccttcagcgcgaaccaggagatccgctacagcgaagttacgccgtaccacgtgacttccgtgtgggttaaaggcgttacgccgc
ctcagtggtggacccaaaccgaagacttcttttacgccccatacgtcccgaatcaaggttggtatgatttgaccaagacctttaac
ggcaaggatgatctgctttgcggtgctgcgaccgcgatcaacatgctgtggtggtggtttgacgtgaacaaagagaagattgag
gagtatctcaagaagcatccggacaaacaaaaaatcatgaaagacgaccaagagctgctagacgtgcgcaaggttatcaat
accaaatgggatcaaacgaatagccacctgttcctgtattttcgtgatattgcgtttccgggtctgtctgcgcgtcgtatcggagtaat
gccggatttggttctgatcatgttcatcatgggttattacctgaatgtttacaaaacgcagaccacggacgtcaaccgtaccccgca
agaaaaggactggcgtggtggcatttttatcgctgtcttcaccttgggtgatccgagcaaatatctgaccagccgtcatgattttaaa
gaaaagaacctgaaggagattagcgacaccatcaaaaaactgttggaggaaggcaaaatgctgggcctgagccacacctat
gctaacgtgcgcattaaccacgtgattaatctgtggggtgcagatttcgactctaatggcctgctcaaggccatctacgtgactgac
agcgactctaacccgtcgatcggcatgttgaagtacttcgtgggcgtgaacagcgcgggtaaggtggcgatttccctgaaacag
attgatgaagataacatcggtgcacaggttctgggcttgttcaccctggacaccggtcaggatctgtggaatcagaccaactaa
Ides^{mt3} protein sequence (340 aa + stop codon) (mutations underlined)
(SEQ ID NO: 18)
RKRCYSTSAVVLAAVTLFALSVDRGVIADSFSANQEIRYSEVTPYHVTSVWVKGVTPPQW
WTQTEDFFYAPYVPNQGWYDLTKTFNGKDDLLCGAATAINMLWWWFDVNKEKIEEYLKKH
PDKQKIMKDDQELLDVRKVINTKWDQTNSHLFLYFRDIAFPGLSARRIGVMPDLVLIMFIMG
YYLNVYKTQTTDVNRTPQEKDWRGGIFIAVFTLGDPSKYLTSRHDFKEKNLKEISDTIKKLLE
EGKMLGLSHTYANVRINHVINLWGADFDSNGLLKAIYVTDSDSNPSIGMLKYFVGVNSAGK
VAISLKQIDEDNIGAQVLGLFTLDTGQDLWNQTN*
Plasmid pGEX-IdeS^{mt4} (6,004 bp)
(SEQ ID NO: 19)
acgttatcgactgcacggtgcaccaatgcttctggcgtcaggcagccatcggaagctgtggtatggctgtgcaggtcgtaaatca
ctgcataattcgtgtcgctcaaggcgcactcccgttctggataatgttttttgcgccgacatcataacggttctggcaaatattctgaa
atgagctgttgacaattaatcatcggctcgtataatgtgtggaattgtgagcggataacaatttcacacaggaaacagtattcatgt
cccctatactaggttattggaaaattaagggccttgtgcaacccactcgacttcttttggaatatcttgaagaaaaatatgaagagc
atttgtatgagcgcgatgaaggtgataaatggcgaaacaaaaagtttgaattgggtttggagtttcccaatcttccttattatattgatg
gtgatgttaaattaacacagtctatggccatcatacgttatatagctgacaagcacaacatgttgggtggttgtccaaaagagcgtg
cagagatttcaatgcttgaaggagcggttttggatattagatacggtgtttcgagaattgcatatagtaaagactttgaaactctcaa
agttgattttcttagcaagctacctgaaatgctgaaaatgttcgaagatcgtttatgtcataaaacatatttaaatggtgatcatgtaac
ccatcctgacttcatgttgtatgacgctcttgatgttgttttatacatggacccaatgtgcctggatgcgttcccaaaattagtttgttttaa
aaaacgtattgaagctatcccacaaattgataagtacttgaaatccagcaagtatatagcatggcctttgcagggctggcaagcc
acgtttggtggtggcgaccatcctccaaaatcggatctggaagttctgttccaggggcccctgggatccaggaaaagatgttattc
aacaagtgctgtagttctggcagcggttactctgttcgccctgtcagttgatcgtggcgtgattgctgactcgttctccgcgaaccaa
gaaatccgttatagcgaagttacgccgtaccacgtgacctccgtgtggaccaagggcgtcaccccgccagcgaaatttaccca
gggtgaagacgtgttccatgcaccgtatgttgcgaatcaaggttggtatgacattaccaagacctttaacggcaaagatgatctgc
tttgcggtgccgctaccgcaattaacatgctgcattggtggtttgaccaaaacaaagagaagattgaagcatacctaaagaaac
acccggacaagcagaaaatcatgttcggcgatcaagagctgctggacgtccgcaaagtgatcaacaccaagtgggatcaga
cgaacagcgaattgtttaattatttccgcgataaagcgtttccgggtctgagcgcgcgtcgcattggtgtcatgccggacttggttctg
gacatgtttatcaacggctattacctgaatgtttataaaactcaaacgaccgacgttaatcgtacctaccaggagaaggaccgcc
gtggtggtatttttgatgcagtattcaccagaggcgatcaaagcaaactgttgacctctcgtcacgatttcaaggaaaaaaacttga
aggagatcagcgatctgatcaaaaaggagttaaccgaaggtaaggcgctcggtttgagccacacctacgcgaacgttcgtatt
aaccatgtcattaacctgtggggtgcggacttcgactccaacggtaatttgaaggcgatctacgtgacggattccgatagcaacg
ccagcattggcatgaaaaaatacttcgtgggtgtgaatagtgctggcaaggtggccatcagcgcgaaggagatcaaagagga
caacatcggcgctcaggttctgggcctgtttactctgagcaccggtcaggattcttggaatcagacgaattaagaattcccgggtc
gactcgagcggccgcatcgtgactgactgacgatctgcctcgcgcgtttcggtgatgacggtgaaaacctctgacacatgcagct
cccggagacggtcacagcttgtctgtaagcggatgccgggagcagacaagcccgtcagggcgcgtcagcgggtgttggcgg
gtgtcggggcgcagccatgacccagtcacgtagcgatagcggagtgtataattcttgaagacgaaagggcctcgtgatacgcct
atttttataggttaatgtcatgataataatggtttcttagacgtcaggtggcacttttcggggaaatgtgcgcggaacccctatttgtttat
ttttctaaatacattcaaatatgtatccgctcatgagacaataaccctgataaatgcttcaataatattgaaaaaggaagagtatga
gtattcaacatttccgtgtcgcccttattcccttttttgcggcattttgccttcctgtttttgctcacccagaaacgctggtgaaagtaaaa
gatgctgaagatcagttgggtgcacgagtgggttacatcgaactggatctcaacagcggtaagatccttgagagttttcgccccg
aagaacgttttccaatgatgagcacttttaaagttctgctatgtggcgcggtattatcccgtgttgacgccgggcaagagcaactcg
gtcgccgcatacactattctcagaatgacttggttgagtactcaccagtcacagaaaagcatcttacggatggcatgacagtaag
agaattatgcagtgctgccataaccatgagtgataacactgcggccaacttacttctgacaacgatcggaggaccgaaggagct
aaccgcttttttgcacaacatgggggatcatgtaactcgccttgatcgttgggaaccggagctgaatgaagccataccaaacgac
gagcgtgacaccacgatgcctgcagcaatggcaacaacgttgcgcaaactattaactggcgaactacttactctagcttcccgg
caacaattaatagactggatggaggcggataaagttgcaggaccacttctgcgctcggcccttccggctggctggtttattgctgat
aaatctggagccggtgagcgtgggtctcgcggtatcattgcagcactggggccagatggtaagccctcccgtatcgtagttatcta
cacgacggggagtcaggcaactatggatgaacgaaatagacagatcgctgagataggtgcctcactgattaagcattggtaac
tgtcagaccaagtttactcatatatactttagattgatttaaaacttcatttttaatttaaaaggatctaggtgaagatcctttttgataatc
tcatgaccaaaatcccttaacgtgagttttcgttccactgagcgtcagaccccgtagaaaagatcaaaggatcttcttgagatcctttt
tttctgcgcgtaatctgctgcttgcaaacaaaaaaaccaccgctaccagcggtggtttgtttgccggatcaagagctaccaactctt
tttccgaaggtaactggcttcagcagagcgcagataccaaatactgtccttctagtgtagccgtagttaggccaccacttcaagaa
ctctgtagcaccgcctacatacctcgctctgctaatcctgttaccagtggctgctgccagtggcgataagtcgtgtcttaccgggttg
gactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacag
gtatccggtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaacgccagcaacg
cggcctttttacggttcctggccttttgctggccttttgctcacatgttctttcctgcgttatcccctgattctgtggataaccgtattaccg
cctttgagtgagctgataccgctcgccgcagccgaacgaccgagcgcagcgagtcagtgagcgaggaagcggaagagcgcct
gatgcggtattttctccttacgcatctgtgcggtatttcacaccgcataaattccgacaccatcgaatggtgcaaaacctttcgcggt
atggcatgatagcgcccggaagagagtcaattcagggtggtgaatgtgaaaccagtaacgttatacgatgtcgcagagtatgcc
ggtgtctcttatcagaccgtttcccgcgtggtgaaccaggccagccacgtttctgcgaaaacgcgggaaaaagtggaagcggc
gatggcggagctgaattacattcccaaccgcgtggcacaacaactggcgggcaaacagtcgttgctgattggcgttgccacctc
cagtctggccctgcacgcgccgtcgcaaattgtcgcggcgattaaatctcgcgccgatcaactgggtgccagcgtggtggtgtcg
atggtagaacgaagcggcgtcgaagcctgtaaagcggcggtgcacaatcttctcgcgcaacgcgtcagtgggctgatcattaa
ctatccgctggatgaccaggatgccattgctgtggaagctgcctgcactaatgttccggcgttatttcttgatgtctctgaccagaca
cccatcaacagtattattttctcccatgaagacggtacgcgactgggcgtggagcatctggtcgcattgggtcaccagcaaatcg
cgctgttagcgggcccattaagttctgtctcggcgcgtctgcgtctggctggctggcataaatatctcactcgcaatcaaattcagcc
gatagcggaacgggaaggcgactggagtgccatgtccggttttcaacaaaccatgcaaatgctgaatgagggcatcgttccca
ctgcgatgctggttgccaacgatcagatggcgctgggcgcaatgcgcgccattaccgagtccgggctgcgcgttggtgcggata
tctcggtagtgggatacgacgataccgaagacagctcatgttatatcccgccgtcaaccaccatcaaacaggattttcgcctgctg
gggcaaaccagcgtggaccgcttgctgcaactctctcagggccaggcggtgaagggcaatcagctgttgcccgtctcactggtg
aaaagaaaaaccaccctggcgcccaatacgcaaaccgcctctccccgcgcgttggccgattcattaatgcagctggcacgac
aggtttcccgactggaaagcgggcagtgagcgcaacgcaattaatgtgagttagctcactcattaggcaccccaggctttacact
ttatgcttccggctcgtatgttgtgtggaattgtgagcggataacaatttcacacaggaaacagctatgaccatgattacggattcac
tggccgtcgttttacaacgtcgtgactgggaaaaccctggcgttacccaacttaatcgccttgcagcacatccccctttcgccagct
ggcgtaatagcgaagaggcccgcaccgatcgcccttcccaacagttgcgcagcctgaatggcgaatggcgctttgcctggtttc
cggcaccagaagcggtgccggaaagctggctggagtgcgatcttcctgaggccgatactgtcgtcgtcccctcaaactggcag
atgcacggttacgatgcgcccatctacaccaacgtaacctatcccattacggtcaatccgccgtttgttcccacggagaatccgac
gggttgttactcgctcacatttaatgttgatgaaagctggctacaggaaggccagacgcgaattatttttgatggcgttggaatt
IdeS^{mt4} DNA sequence (1,023 bp)
(SEQ ID NO: 20)
aggaaaagatgttattcaacaagtgctgtagttctggcagcggttactctgttcgccctgtcagttgatcgtggcgtgattgctgactc
gttctccgcgaaccaagaaatccgttatagcgaagttacgccgtaccacgtgacctccgtgtggaccaagggcgtcaccccgc
cagcgaaatttacccagggtgaagacgtgttccatgcaccgtatgttgcgaatcaaggttggtatgacattaccaagacctttaac
ggcaaagatgatctgctttgcggtgccgctaccgcaattaacatgctgcattggtggtttgaccaaaacaaagagaagattgaag
catacctaaagaaacacccggacaagcagaaaatcatgttcggcgatcaagagctgctggacgtccgcaaagtgatcaaca
ccaagtgggatcagacgaacagcgaattgtttaattatttccgcgataaagcgtttccgggtctgagcgcgcgtcgcattggtgtc
atgccggacttggttctggacatgtttatcaacggctattacctgaatgtttataaaactcaaacgaccgacgttaatcgtacctacc
aggagaaggaccgccgtggtggtatttttgatgcagtattcaccagaggcgatcaaagcaaactgttgacctctcgtcacgatttc
aaggaaaaaaacttgaaggagatcagcgatctgatcaaaaaggagttaaccgaaggtaaggcgctcggtttgagccacacct
acgcgaacgttcgtattaaccatgtcattaacctgtggggtgcggacttcgactccaacggtaatttgaaggcgatctacgtgacg
gattccgatagcaacgccagcattggcatgaaaaaatacttcgtgggtgtgaatagtgctggcaaggtggccatcagcgcgaa
ggagatcaaagaggacaacatcggcgctcaggttctgggcctgtttactctgagcaccggtcaggattcttggaatcagacgaa
ttaa
IdeS^{mt4} Protein sequence (340 aa + stop codon) (mutations underlined)
(SEQ ID NO: 21)
RKRCYSTSAVVLAAVTLFALSVDRGVIADSFSANQEIRYSEVTPYHVTSVWTKGVTPPAKFT
QGEDVFHAPYVANQGWYDITKTFNGKDDLLCGAATAINMLHWWFDQNKEKIEAYLKKHPD
KQKIMFGDQELLDVRKVINTKWDQTNSELFNYFRDKAFPGLSARRIGVMPDLVLDMFINGY
YLNVYKTQTTDVNRTYQEKDRRGGIFDAVFTRGDQSKLLTSRHDFKEKNLKEISDLIKKELT
EGKALGLSHTYANVRINHVINLWGADFDSNGNLKAIYVTDSDSNASIGMKKYFVGVNSAGK
VAISAKEIKEDNIGAQVLGLFTLSTGQDSWNQTN*
Plasmid pGEX-IdeS^{mt5} (6,004 bp)
(SEQ ID NO: 22)
acgttatcgactgcacggtgcaccaatgcttctggcgtcaggcagccatcggaagctgtggtatggctgtgcaggtcgtaaatca
ctgcataattcgtgtcgctcaaggcgcactcccgttctggataatgttttttgcgccgacatcataacggttctggcaaatattctgaa
atgagctgttgacaattaatcatcggctcgtataatgtgtggaattgtgagcggataacaatttcacacaggaaacagtattcatgt
cccctatactaggttattggaaaattaagggccttgtgcaacccactcgacttcttttggaatatcttgaagaaaaatatgaagagc
atttgtatgagcgcgatgaaggtgataaatggcgaaacaaaaagtttgaattgggtttggagtttcccaatcttccttattatattgatg
gtgatgttaaattaacacagtctatggccatcatacgttatatagctgacaagcacaacatgttgggtggttgtccaaaagagcgtg
cagagatttcaatgcttgaaggagcggttttggatattagatacggtgtttcgagaattgcatatagtaaagactttgaaactctcaa
agttgattttcttagcaagctacctgaaatgctgaaaatgttcgaagatcgtttatgtcataaaacatatttaaatggtgatcatgtaac
ccatcctgacttcatgttgtatgacgctcttgatgttgttttatacatggacccaatgtgcctggatgcgttcccaaaattagtttgttttaa
aaaacgtattgaagctatcccacaaattgataagtacttgaaatccagcaagtatatagcatggcctttgcagggctggcaagcc
acgtttggtggtggcgaccatcctccaaaatcggatctggaagttctgttccaggggcccctgggatccaggaaaagatgttattc
aacaagtgctgtagtactggctgctgttactctgttcgccttaagcgttgaccgcggcgtcatcgcggattcctttagcgcgaacca
agagattcgttatagcgaggtgacgccgtaccacgttgaagaagtttggaccaaaggtgttacgccaccggctaaattcaccca
gggtgaagacgtcttccatgcaccgtacgttgcaaaccagggctggtatgatatcaccaagacctttaatggtaaagatgatctg
ctttgcggtgctgcgaccgcaggtaatatgctgcattggtggttcgaccagaataaagagaagattgaagcatatttgaaaaagc
acccggacaagcaaaaaatcatgttcggcgatcaagagctgcttgatgtgcgcaaggtgattaacaccaagggcgaccaaac
caattccgaactgtttaactacttccgcgataaggcgtttccgggtttgtcggcgcgtcgtatcggggtgatgccggatttggttctgg
acatgttcatcaacggctattacctgaatgtgtataaaacccaaaccaccgacgtgaatcgtacctaccaggagaaggacaga
cgtggcggtatttttgacgccgtgttcacccgtggtgaccaaagcaaactgttgaccagccgtcacgattttaaagagctgaacct
gaagcaaatcagcgacatcatccgcaaagagttgctggaaggtaaggccctcggcctgagccatacgtacgcaaacgtgcgt
attaaccacgtgatcaacctgtggggtgcggtgttcgactctaatggcttgctcaaggcgatttatgtcactgattctgactccaacg
ccagcattggcatgaaaaaatactttgttgacatcaacagcgcgggcaagctggcgatctctgcgaaacagattgatgaagata
atattggtgcgcaggttctgggtttgtttacactgtcgaccggtcaggattcttggaaccagacgaactaagaattcccgggtcgac
tcgagcggccgcatcgtgactgactgacgatctgcctcgcgcgtttcggtgatgacggtgaaaacctctgacacatgcagctccc
ggagacggtcacagcttgtctgtaagcggatgccgggagcagacaagcccgtcagggcgcgtcagcgggtgttggcgggtgt
cggggcgcagccatgacccagtcacgtagcgatagcggagtgtataattcttgaagacgaaagggcctcgtgatacgcctatttt
tataggttaatgtcatgataataatggtttcttagacgtcaggtggcacttttcggggaaatgtgcgcggaacccctatttgtttatttttc
taaatacattcaaatatgtatccgctcatgagacaataaccctgataaatgcttcaataatattgaaaaaggaagagtatgagtatt
caacatttccgtgtcgcccttattcccttttttgcggcattttgccttcctgtttttgctcacccagaaacgctggtgaaagtaaaagatgc
tgaagatcagttgggtgcacgagtgggttacatcgaactggatctcaacagcggtaagatccttgagagttttcgccccgaagaa
cgttttccaatgatgagcacttttaaagttctgctatgtggcgcggtattatcccgtgttgacgccgggcaagagcaactcggtcgcc
gcatacactattctcagaatgacttggttgagtactcaccagtcacagaaaagcatcttacggatggcatgacagtaagagaatt
atgcagtgctgccataaccatgagtgataacactgcggccaacttacttctgacaacgatcggaggaccgaaggagctaaccg
cttttttgcacaacatgggggatcatgtaactcgccttgatcgttgggaaccggagctgaatgaagccataccaaacgacgagcg
tgacaccacgatgcctgcagcaatggcaacaacgttgcgcaaactattaactggcgaactacttactctagcttcccggcaaca
attaatagactggatggaggcggataaagttgcaggaccacttctgcgctcggcccttccggctggctggtttattgctgataaatc
tggagccggtgagcgtgggtctcgcggtatcattgcagcactggggccagatggtaagccctcccgtatcgtagttatctacacg
acggggagtcaggcaactatggatgaacgaaatagacagatcgctgagataggtgcctcactgattaagcattggtaactgtca
gaccaagtttactcatatatactttagattgatttaaaacttcatttttaatttaaaaggatctaggtgaagatcctttttgataatctcatg
accaaaatcccttaacgtgagttttcgttccactgagcgtcagaccccgtagaaaagatcaaaggatcttcttgagatcctttttttct
gcgcgtaatctgctgcttgcaaacaaaaaaaccaccgctaccagcggtggtttgtttgccggatcaagagctaccaactctttttcc
gaaggtaactggcttcagcagagcgcagataccaaatactgtccttctagtgtagccgtagttaggccaccacttcaagaactct
gtagcaccgcctacatacctcgctctgctaatcctgttaccagtggctgctgccagtggcgataagtcgtgtcttaccgggttggact
caagacgatagttaccggataaggcgcagcggtcgggctgaacggggggttcgtgcacacagcccagcttggagcgaacga
cctacaccgaactgagatacctacagcgtgagctatgagaaagcgccacgcttcccgaagggagaaaggcggacaggtatc
cggtaagcggcagggtcggaacaggagagcgcacgagggagcttccagggggaaacgcctggtatctttatagtcctgtcgg
gtttcgccacctctgacttgagcgtcgatttttgtgatgctcgtcaggggggcggagcctatggaaaaacgccagcaacgcggcc
tttttacggttcctggccttttgctggccttttgctcacatgttctttcctgcgttatcccctgattctgtggataaccgtattaccgccttt
gagtgagctgataccgctcgccgcagccgaacgaccgagcgcagcgagtcagtgagcgaggaagcggaagagcgcctgatgc
ggtattttctccttacgcatctgtgcggtatttcacaccgcataaattccgacaccatcgaatggtgcaaaacctttcgcggtatggc
atgatagcgcccggaagagagtcaattcagggtggtgaatgtgaaaccagtaacgttatacgatgtcgcagagtatgccggtgt
ctcttatcagaccgtttcccgcgtggtgaaccaggccagccacgtttctgcgaaaacgcgggaaaaagtggaagcggcgatgg
cggagctgaattacattcccaaccgcgtggcacaacaactggcgggcaaacagtcgttgctgattggcgttgccacctccagtct
ggccctgcacgcgccgtcgcaaattgtcgcggcgattaaatctcgcgccgatcaactgggtgccagcgtggtggtgtcgatggt
agaacgaagcggcgtcgaagcctgtaaagcggcggtgcacaatcttctcgcgcaacgcgtcagtgggctgatcattaactatc
cgctggatgaccaggatgccattgctgtggaagctgcctgcactaatgttccggcgttatttcttgatgtctctgaccagacacccat
caacagtattattttctcccatgaagacggtacgcgactgggcgtggagcatctggtcgcattgggtcaccagcaaatcgcgctgt
tagcgggcccattaagttctgtctcggcgcgtctgcgtctggctggctggcataaatatctcactcgcaatcaaattcagccgatag
cggaacgggaaggcgactggagtgccatgtccggttttcaacaaaccatgcaaatgctgaatgagggcatcgttcccactgcg
atgctggttgccaacgatcagatggcgctgggcgcaatgcgcgccattaccgagtccgggctgcgcgttggtgcggatatctcg
gtagtgggatacgacgataccgaagacagctcatgttatatcccgccgtcaaccaccatcaaacaggattttcgcctgctgggg
caaaccagcgtggaccgcttgctgcaactctctcagggccaggcggtgaagggcaatcagctgttgcccgtctcactggtgaaa
agaaaaaccaccctggcgcccaatacgcaaaccgcctctccccgcgcgttggccgattcattaatgcagctggcacgacaggt
ttcccgactggaaagcgggcagtgagcgcaacgcaattaatgtgagttagctcactcattaggcaccccaggctttacactttatg
cttccggctcgtatgttgtgtggaattgtgagcggataacaatttcacacaggaaacagctatgaccatgattacggattcactggc
cgtcgttttacaacgtcgtgactgggaaaaccctggcgttacccaacttaatcgccttgcagcacatccccctttcgccagctggcg
taatagcgaagaggcccgcaccgatcgcccttcccaacagttgcgcagcctgaatggcgaatggcgctttgcctggtttccggc
accagaagcggtgccggaaagctggctggagtgcgatcttcctgaggccgatactgtcgtcgtcccctcaaactggcagatgc
acggttacgatgcgcccatctacaccaacgtaacctatcccattacggtcaatccgccgtttgttcccacggagaatccgacggg
ttgttactcgctcacatttaatgttgatgaaagctggctacaggaaggccagacgcgaattatttttgatggcgttggaatt
IdeS^{mt5} DNA sequence (1,023 bp)
(SEQ ID NO: 23)
aggaaaagatgttattcaacaagtgctgtagtactggctgctgttactctgttcgccttaagcgttgaccgcggcgtcatcgcggatt
cctttagcgcgaaccaagagattcgttatagcgaggtgacgccgtaccacgttgaagaagtttggaccaaaggtgttacgccac
cggctaaattcacccagggtgaagacgtcttccatgcaccgtacgttgcaaaccagggctggtatgatatcaccaagacctttaa
tggtaaagatgatctgctttgcggtgctgcgaccgcaggtaatatgctgcattggtggttcgaccagaataaagagaagattgaa
gcatatttgaaaaagcacccggacaagcaaaaaatcatgttcggcgatcaagagctgcttgatgtgcgcaaggtgattaacac
caagggcgaccaaaccaattccgaactgtttaactacttccgcgataaggcgtttccgggtttgtcggcgcgtcgtatcggggtga
tgccggatttggttctggacatgttcatcaacggctattacctgaatgtgtataaaacccaaaccaccgacgtgaatcgtacctacc
aggagaaggacagacgtggcggtatttttgacgccgtgttcacccgtggtgaccaaagcaaactgttgaccagccgtcacgattt
taaagagctgaacctgaagcaaatcagcgacatcatccgcaaagagttgctggaaggtaaggccctcggcctgagccatacg
tacgcaaacgtgcgtattaaccacgtgatcaacctgtggggtgcggtgttcgactctaatggcttgctcaaggcgatttatgtcactg
attctgactccaacgccagcattggcatgaaaaaatactttgttgacatcaacagcgcgggcaagctggcgatctctgcgaaac
agattgatgaagataatattggtgcgcaggttctgggtttgtttacactgtcgaccggtcaggattcttggaaccagacgaactaa
IdeS^{mt5} Protein sequence (340 aa) (mutations underlined)
(SEQ ID NO: 24)
RKRCYSTSAVVLAAVTLFALSVDRGVIADSFSANQEIRYSEVTPYHVEEVWTKGVTPPAKFT
QGEDVFHAPYVANQGWYDITKTFNGKDDLLCGAATAGNMLHWWFDQNKEKIEAYLKKHP
DKQKIMFGDQELLDVRKVINTKGDQTNSELFNYFRDKAFPGLSARRIGVMPDLVLDMFING
YYLNVYKTQTTDVNRTYQEKDRRGGIFDAVFTRGDQSKLLTSRHDFKELNLKQISDIIRKELL
EGKALGLSHTYANVRINHVINLWGAVFDSNGLLKAIYVTDSDSNASIGMKKYFVDINSAGKL
AISAKQIDEDNIGAQVLGLFTLSTGQDSWNQTN
Plasmid pGEX-IdeS^{mt6} (6,004 bp)
(SEQ ID NO: 25)
acgttatcgactgcacggtgcaccaatgcttctggcgtcaggcagccatcggaagctgtggtatggctgtgcaggtcgtaaatca
ctgcataattcgtgtcgctcaaggcgcactcccgttctggataatgttttttgcgccgacatcataacggttctggcaaatattctgaa
atgagctgttgacaattaatcatcggctcgtataatgtgtggaattgtgagcggataacaatttcacacaggaaacagtattcatgt
cccctatactaggttattggaaaattaagggccttgtgcaacccactcgacttcttttggaatatcttgaagaaaaatatgaagagc
atttgtatgagcgcgatgaaggtgataaatggcgaaacaaaaagtttgaattgggtttggagtttcccaatcttccttattatattgatg
gtgatgttaaattaacacagtctatggccatcatacgttatatagctgacaagcacaacatgttgggtggttgtccaaaagagcgtg
cagagatttcaatgcttgaaggagcggttttggatattagatacggtgtttcgagaattgcatatagtaaagactttgaaactctcaa
agttgattttcttagcaagctacctgaaatgctgaaaatgttcgaagatcgtttatgtcataaaacatatttaaatggtgatcatgtaac
ccatcctgacttcatgttgtatgacgctcttgatgttgttttatacatggacccaatgtgcctggatgcgttcccaaaattagtttgttttaa
aaaacgtattgaagctatcccacaaattgataagtacttgaaatccagcaagtatatagcatggcctttgcagggctggcaagcc
acgtttggtggtggcgaccatcctccaaaatcggatctggaagttctgttccaggggcccctgggatccaggaaaagatgttattc
aacaagtgctgtagtactggcagcggtgaccttgtttgcactgtcggttgatcgtggggtcatcgccgattcttttagcgcgaatcaa
gaaattcgttatagcgaagtgaccccgtaccatgtttggagcgtgtggaccaaaggcgttacgccgccagcgaaattcacgcag
ggtgaagacgttttccacgcaccgtacgtggcgaaccaaggttggtatgatatcaccaaaacctttaacggcaaggacgatttac
tttgcggtgcggctaccgcaggtaatatgctgcattggtggtttgatcaaaacaaagagaagattgaagcgtatctgaagaaaca
cccggataagcagaaaatcatgtttggtgaccaagagctgttagacgtgcgcaaagtcattaacaccaagggcgaccaaacg
aacagcgaactgttcaactatttccgcgataaggcatttccgggtttgtctgcccgtcgtattggcgtgatgccggacctggttctgg
atatgttcatcaacggttattacttgaatgtttataaaacccagaccaccgatgtgaatagaacataccaggagaaggaccgccg
tggtggtatttttgacgctgtgttcacgcgtggcgaccaaagcaagctgttgaccagccgtcatgatttcaaagagaagaacctga
aagagatcagcgacataatcctgaaggagctcttggaaggtaaggctctgggcctgtctcacacctacgcgaacgttcgcatta
accacgtgatcaacctgtggggcgcggacttcgactccaatggcttgcttaaggccatctacgttactgactccgactccaacgc
cagcattggcatgaaaaagtactttgttggtgtcaatagcgcgggtaaagttgcgattagcgcgaaagaaatcaaagaggataa
catcggcgctcaggtgctgggcctgttcactctgtcgaccggtcaggattcatggaaccagaccaattaagaattcccgggtcga
ctcgagcggccgcatcgtgactgactgacgatctgcctcgcgcgtttcggtgatgacggtgaaaacctctgacacatgcagctcc
cggagacggtcacagcttgtctgtaagcggatgccgggagcagacaagcccgtcagggcgcgtcagcgggtgttggcgggtg
tcggggcgcagccatgacccagtcacgtagcgatagcggagtgtataattcttgaagacgaaagggcctcgtgatacgcctattt
ttataggttaatgtcatgataataatggtttcttagacgtcaggtggcacttttcggggaaatgtgcgcggaacccctatttgtttattttt
ctaaatacattcaaatatgtatccgctcatgagacaataaccctgataaatgcttcaataatattgaaaaaggaagagtatgagta
ttcaacatttccgtgtcgcccttattcccttttttgcggcattttgccttcctgtttttgctcacccagaaacgctggtgaaagtaaaagat
gctgaagatcagttgggtgcacgagtgggttacatcgaactggatctcaacagcggtaagatccttgagagttttcgccccgaag
aacgttttccaatgatgagcacttttaaagttctgctatgtggcgcggtattatcccgtgttgacgccgggcaagagcaactcggtcg
ccgcatacactattctcagaatgacttggttgagtactcaccagtcacagaaaagcatcttacggatggcatgacagtaagaga
attatgcagtgctgccataaccatgagtgataacactgcggccaacttacttctgacaacgatcggaggaccgaaggagctaac
cgcttttttgcacaacatgggggatcatgtaactcgccttgatcgttgggaaccggagctgaatgaagccataccaaacgacgag
cgtgacaccacgatgcctgcagcaatggcaacaacgttgcgcaaactattaactggcgaactacttactctagcttcccggcaa
caattaatagactggatggaggcggataaagttgcaggaccacttctgcgctcggcccttccggctggctggtttattgctgataa
atctggagccggtgagcgtgggtctcgcggtatcattgcagcactggggccagatggtaagccctcccgtatcgtagttatctaca
cgacggggagtcaggcaactatggatgaacgaaatagacagatcgctgagataggtgcctcactgattaagcattggtaactgt
cagaccaagtttactcatatatactttagattgatttaaaacttcatttttaatttaaaaggatctaggtgaagatcctttttgataatctca
tgaccaaaatcccttaacgtgagttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatcaagagctaccaactcttttt
ccgaaggtaactggcttcagcagagcgcagataccaaatactgtccttctagtgtagccgtagttaggccaccacttcaagaact
ctgtagcaccgcctacatacctcgctctgctaatcctgttaccagtggctgctgccagtggcgataagtcgtgtcttaccgggttgga
ctcaagacgatagttaccggataaggcgcagcggtcgggctgaacggggggttcgtgcacacagcccagcttggagcgaac
gacctacaccgaactgagatacctacagcgtgagctatgagaaagcgccacgcttcccgaagggagaaaggcggacaggt
atccggtaagcggcagggtcggaacaggagagcgcacgagggagcttccagggggaaacgcctggtatctttatagtcctgt
cgggtttcgccacctctgacttgagcgtcgatttttgtgatgctcgtcaggggggcggagcctatggaaaaacgccagcaacgcg
gcctttttacggttcctggccttttgctggccttttgctcacatgttctttcctgcgttatcccctgattctgtggataaccgtattaccgcc
tttgagtgagctgataccgctcgccgcagccgaacgaccgagcgcagcgagtcagtgagcgaggaagcggaagagcgcctga
tgcggtattttctccttacgcatctgtgcggtatttcacaccgcataaattccgacaccatcgaatggtgcaaaacctttcgcggtatg
gcatgatagcgcccggaagagagtcaattcagggtggtgaatgtgaaaccagtaacgttatacgatgtcgcagagtatgccggt
gtctcttatcagaccgtttcccgcgtggtgaaccaggccagccacgtttctgcgaaaacgcgggaaaaagtggaagcggcgat
ggcggagctgaattacattcccaaccgcgtggcacaacaactggcgggcaaacagtcgttgctgattggcgttgccacctccag
tctggccctgcacgcgccgtcgcaaattgtcgcggcgattaaatctcgcgccgatcaactgggtgccagcgtggtggtgtcgatg
gtagaacgaagcggcgtcgaagcctgtaaagcggcggtgcacaatcttctcgcgcaacgcgtcagtgggctgatcattaactat
ccgctggatgaccaggatgccattgctgtggaagctgcctgcactaatgttccggcgttatttcttgatgtctctgaccagacaccca
tcaacagtattattttctcccatgaagacggtacgcgactgggcgtggagcatctggtcgcattgggtcaccagcaaatcgcgctg
ttagcgggcccattaagttctgtctcggcgcgtctgcgtctggctggctggcataaatatctcactcgcaatcaaattcagccgata
gcggaacgggaaggcgactggagtgccatgtccggttttcaacaaaccatgcaaatgctgaatgagggcatcgttcccactgc
gatgctggttgccaacgatcagatggcgctgggcgcaatgcgcgccattaccgagtccgggctgcgcgttggtgcggatatctc
ggtagtgggatacgacgataccgaagacagctcatgttatatcccgccgtcaaccaccatcaaacaggattttcgcctgctggg
gcaaaccagcgtggaccgcttgctgcaactctctcagggccaggcggtgaagggcaatcagctgttgcccgtctcactggtgaa
aagaaaaaccaccctggcgcccaatacgcaaaccgcctctccccgcgcgttggccgattcattaatgcagctggcacgacag
gtttcccgactggaaagcgggcagtgagcgcaacgcaattaatgtgagttagctcactcattaggcaccccaggctttacacttta
tgcttccggctcgtatgttgtgtggaattgtgagcggataacaatttcacacaggaaacagctatgaccatgattacggattcactg
gccgtcgttttacaacgtcgtgactgggaaaaccctggcgttacccaacttaatcgccttgcagcacatccccctttcgccagctgg
cgtaatagcgaagaggcccgcaccgatcgcccttcccaacagttgcgcagcctgaatggcgaatggcgctttgcctggtttccg
gcaccagaagcggtgccggaaagctggctggagtgcgatcttcctgaggccgatactgtcgtcgtcccctcaaactggcagat
gcacggttacgatgcgcccatctacaccaacgtaacctatcccattacggtcaatccgccgtttgttcccacggagaatccgacg
ggttgttactcgctcacatttaatgttgatgaaagctggctacaggaaggccagacgcgaattatttttgatggcgttggaatt
Ides^{mt6} DNA sequence (1,023 bp)
(SEQ ID NO: 26)
aggaaaagatgttattcaacaagtgctgtagtactggcagcggtgaccttgtttgcactgtcggttgatcgtggggtcatcgccgatt
cttttagcgcgaatcaagaaattcgttatagcgaagtgaccccgtaccatgtttggagcgtgtggaccaaaggcgttacgccgcc
agcgaaattcacgcagggtgaagacgttttccacgcaccgtacgtggcgaaccaaggttggtatgatatcaccaaaacctttaa
cggcaaggacgatttactttgcggtgcggctaccgcaggtaatatgctgcattggtggtttgatcaaaacaaagagaagattgaa
gcgtatctgaagaaacacccggataagcagaaaatcatgtttggtgaccaagagctgttagacgtgcgcaaagtcattaacac
caagggcgaccaaacgaacagcgaactgttcaactatttccgcgataaggcatttccgggtttgtctgcccgtcgtattggcgtga
tgccggacctggttctggatatgttcatcaacggttattacttgaatgtttataaaacccagaccaccgatgtgaatagaacatacc
aggagaaggaccgccgtggtggtatttttgacgctgtgttcacgcgtggcgaccaaagcaagctgttgaccagccgtcatgatttc
aaagagaagaacctgaaagagatcagcgacataatcctgaaggagctcttggaaggtaaggctctgggcctgtctcacacct
acgcgaacgttcgcattaaccacgtgatcaacctgtggggcgcggacttcgactccaatggcttgcttaaggccatctacgttact
gactccgactccaacgccagcattggcatgaaaaagtactttgttggtgtcaatagcgcgggtaaagttgcgattagcgcgaaa
gaaatcaaagaggataacatcggcgctcaggtgctgggcctgttcactctgtcgaccggtcaggattcatggaaccagaccaat
taa
Ides^{mt6} Protein sequence (340 aa) (mutations underlined)
(SEQ ID NO: 27)
RKRCYSTSAVVLAAVTLFALSVDRGVIADSFSANQEIRYSEVTPYHVWSVWTKGVTPPAKF
TQGEDVFHAPYVANQGWYDITKTFNGKDDLLCGAATAGNMLHWWFDQNKEKIEAYLKKH
PDKQKIMFGDQELLDVRKVINTKGDQTNSELFNYFRDKAFPGLSARRIGVMPDLVLDMFIN
GYYLNVYKTQTTDVNRTYQEKDRRGGIFDAVFTRGDQSKLLTSRHDFKEKNLKEISDIILKEL
LEGKALGLSHTYANVRINHVINLWGADFDSNGLLKAIYVTDSDSNASIGMKKYFVGVNSAGK
VAISAKEIKEDNIGAQVLGLFTLSTGQDSWNQTN*
Plasmid pGEX-IdeS^{mt7} (6,004 bp)
(SEQ ID NO: 28)
acgttatcgactgcacggtgcaccaatgcttctggcgtcaggcagccatcggaagctgtggtatggctgtgcaggtcgtaaatca
ctgcataattcgtgtcgctcaaggcgcactcccgttctggataatgttttttgcgccgacatcataacggttctggcaaatattctgaa
atgagctgttgacaattaatcatcggctcgtataatgtgtggaattgtgagcggataacaatttcacacaggaaacagtattcatgt
cccctatactaggttattggaaaattaagggccttgtgcaacccactcgacttcttttggaatatcttgaagaaaaatatgaagagc
atttgtatgagcgcgatgaaggtgataaatggcgaaacaaaaagtttgaattgggtttggagtttcccaatcttccttattatattgatg
gtgatgttaaattaacacagtctatggccatcatacgttatatagctgacaagcacaacatgttgggtggttgtccaaaagagcgtg
cagagatttcaatgcttgaaggagcggttttggatattagatacggtgtttcgagaattgcatatagtaaagactttgaaactctcaa
agttgattttcttagcaagctacctgaaatgctgaaaatgttcgaagatcgtttatgtcataaaacatatttaaatggtgatcatgtaac
ccatcctgacttcatgttgtatgacgctcttgatgttgttttatacatggacccaatgtgcctggatgcgttcccaaaattagtttgttttaa
aaaacgtattgaagctatcccacaaattgataagtacttgaaatccagcaagtatatagcatggcctttgcagggctggcaagcc
acgtttggtggtggcgaccatcctccaaaatcggatctggaagttctgttccaggggcccctgggatccaggaaaagatgttattc
aacaagtgctgtagttctggcggctgtgactctcttcgccttgtctgttgatagaggcgttatcgcggattcatttagcgccaaccaag
aaattcgttatagcgaagttactccgtaccacgtgacttccgtgtggaccaagggcgtgaccccaccggcaaaattcacgcaag
gtgaagacgtgtttcatgcgccgtatgttgcgaaccagggctggtatgatatcaccaagaccttcaatggcaaagatgatttgcttt
gcggtgctgctaccgcaggcaacatgctgcattggtggtttgaccagaataaagagaagattgaagaatacttgaagaagcac
ccggactacattaaaatcatgtttggtgatcaagagctgctggacgtgcgtaaagtgattaacaccaagggagaccaaaccaat
agcgaactgttcaactacttccgcgataaagcgtttccgggtctgagcgcgcgtcgcattggcgtgatgccggacttggttctggat
atgttcatcatgggttattacctaaatgtctataaaacccagaccaccgatgtgaaccgcacctaccaggagaaggaccgtcgtg
gtggtatctttatcgcggtgttcacgttaggcgaccaaagcaaactgctcacgagccgtcacgacttcaaggagaagaacctga
aagagatcagcgacctgattaaaaaagagttgaccgaaggtaaggcgctgggcttgtcccatacctacgcaaatgttcgtatta
accacgttatcaacctttggggtgcggacttcgattctaacggtaatctgaaggccatctacgtgacggattctgactccaacgcc
agcattggcatgaaaaagtattttgttggtgtcaactcggcgggtaaagtagcaattagcgctaaagagatcaaggaggacaac
atcggcgcgcaggttctgggtctgttcacgctgtccaccggtcaggatctgtggaatatgaccctgtaagaattcccgggtcgact
cgagcggccgcatcgtgactgactgacgatctgcctcgcgcgtttcggtgatgacggtgaaaacctctgacacatgcagctccc
ggagacggtcacagcttgtctgtaagcggatgccgggagcagacaagcccgtcagggcgcgtcagcgggtgttggcgggtgt
cggggcgcagccatgacccagtcacgtagcgatagcggagtgtataattcttgaagacgaaagggcctcgtgatacgcctatttt
tataggttaatgtcatgataataatggtttcttagacgtcaggtggcacttttcggggaaatgtgcgcggaacccctatttgtttatttttc
taaatacattcaaatatgtatccgctcatgagacaataaccctgataaatgcttcaataatattgaaaaaggaagagtatgagtatt
caacatttccgtgtcgcccttattcccttttttgcggcattttgccttcctgtttttgctcacccagaaacgctggtgaaagtaaaagatgc
tgaagatcagttgggtgcacgagtgggttacatcgaactggatctcaacagcggtaagatccttgagagttttcgccccgaagaa
cgttttccaatgatgagcacttttaaagttctgctatgtggcgcggtattatcccgtgttgacgccgggcaagagcaactcggtcgcc
gcatacactattctcagaatgacttggttgagtactcaccagtcacagaaaagcatcttacggatggcatgacagtaagagaatt
atgcagtgctgccataaccatgagtgataacactgcggccaacttacttctgacaacgatcggaggaccgaaggagctaaccg
cttttttgcacaacatgggggatcatgtaactcgccttgatcgttgggaaccggagctgaatgaagccataccaaacgacgagcg
tgacaccacgatgcctgcagcaatggcaacaacgttgcgcaaactattaactggcgaactacttactctagcttcccggcaaca
attaatagactggatggaggcggataaagttgcaggaccacttctgcgctcggcccttccggctggctggtttattgctgataaatc
tggagccggtgagcgtgggtctcgcggtatcattgcagcactggggccagatggtaagccctcccgtatcgtagttatctacacg
acggggagtcaggcaactatggatgaacgaaatagacagatcgctgagataggtgcctcactgattaagcattggtaactgtca
gaccaagtttactcatatatactttagattgatttaaaacttcatttttaatttaaaaggatctaggtgaagatcctttttgataatctcatg
accaaaatcccttaacgtgagttttcgttccactgagcgtcagaccccgtagaaaagatcaaaggatcttcttgagatcctttttttct
gcgcgtaatctgctgcttgcaaacaaaaaaaccaccgctaccagcggtggtttgtttgccggatcaagagctaccaactctttttcc
gaaggtaactggcttcagcagagcgcagataccaaatactgtccttctagtgtagccgtagttaggccaccacttcaagaactct
gtagcaccgcctacatacctcgctctgctaatcctgttaccagtggctgctgccagtggcgataagtcgtgtcttaccgggttggact
caagacgatagttaccggataaggcgcagcggtcgggctgaacggggggttcgtgcacacagcccagcttggagcgaacga
cctacaccgaactgagatacctacagcgtgagctatgagaaagcgccacgcttcccgaagggagaaaggcggacaggtatc
cggtaagcggcagggtcggaacaggagagcgcacgagggagcttccagggggaaacgcctggtatctttatagtcctgtcgg
gtttcgccacctctgacttgagcgtcgatttttgtgatgctcgtcaggggggcggagcctatggaaaaacgccagcaacgcggcc
tttttacggttcctggccttttgctggccttttgctcacatgttctttcctgcgttatcccctgattctgtggataaccgtattaccgccttt
gagtgagctgataccgctcgccgcagccgaacgaccgagcgcagcgagtcagtgagcgaggaagcggaagagcgcctgatgc
ggtattttctccttacgcatctgtgcggtatttcacaccgcataaattccgacaccatcgaatggtgcaaaacctttcgcggtatggc
atgatagcgcccggaagagagtcaattcagggtggtgaatgtgaaaccagtaacgttatacgatgtcgcagagtatgccggtgt
ctcttatcagaccgtttcccgcgtggtgaaccaggccagccacgtttctgcgaaaacgcgggaaaaagtggaagcggcgatgg
cggagctgaattacattcccaaccgcgtggcacaacaactggcgggcaaacagtcgttgctgattggcgttgccacctccagtct
ggccctgcacgcgccgtcgcaaattgtcgcggcgattaaatctcgcgccgatcaactgggtgccagcgtggtggtgtcgatggt
agaacgaagcggcgtcgaagcctgtaaagcggcggtgcacaatcttctcgcgcaacgcgtcagtgggctgatcattaactatc
cgctggatgaccaggatgccattgctgtggaagctgcctgcactaatgttccggcgttatttcttgatgtctctgaccagacacccat
caacagtattattttctcccatgaagacggtacgcgactgggcgtggagcatctggtcgcattgggtcaccagcaaatcgcgctgt
tagcgggcccattaagttctgtctcggcgcgtctgcgtctggctggctggcataaatatctcactcgcaatcaaattcagccgatag
cggaacgggaaggcgactggagtgccatgtccggttttcaacaaaccatgcaaatgctgaatgagggcatcgttcccactgcg
atgctggttgccaacgatcagatggcgctgggcgcaatgcgcgccattaccgagtccgggctgcgcgttggtgcggatatctcg
gtagtgggatacgacgataccgaagacagctcatgttatatcccgccgtcaaccaccatcaaacaggattttcgcctgctgggg
caaaccagcgtggaccgcttgctgcaactctctcagggccaggcggtgaagggcaatcagctgttgcccgtctcactggtgaaa
agaaaaaccaccctggcgcccaatacgcaaaccgcctctccccgcgcgttggccgattcattaatgcagctggcacgacaggt
ttcccgactggaaagcgggcagtgagcgcaacgcaattaatgtgagttagctcactcattaggcaccccaggctttacactttatg
cttccggctcgtatgttgtgtggaattgtgagcggataacaatttcacacaggaaacagctatgaccatgattacggattcactggc
cgtcgttttacaacgtcgtgactgggaaaaccctggcgttacccaacttaatcgccttgcagcacatccccctttcgccagctggcg
taatagcgaagaggcccgcaccgatcgcccttcccaacagttgcgcagcctgaatggcgaatggcgctttgcctggtttccggc
accagaagcggtgccggaaagctggctggagtgcgatcttcctgaggccgatactgtcgtcgtcccctcaaactggcagatgc
acggttacgatgcgcccatctacaccaacgtaacctatcccattacggtcaatccgccgtttgttcccacggagaatccgacggg
ttgttactcgctcacatttaatgttgatgaaagctggctacaggaaggccagacgcgaattatttttgatggcgttggaatt
IdeS^{mt7} DNA sequence (6,004 bp)
(SEQ ID NO: 29)
aggaaaagatgttattcaacaagtgctgtagttctggcggctgtgactctcttcgccttgtctgttgatagaggcgttatcgcggattc
atttagcgccaaccaagaaattcgttatagcgaagttactccgtaccacgtgacttccgtgtggaccaagggcgtgaccccaccg
gcaaaattcacgcaaggtgaagacgtgtttcatgcgccgtatgttgcgaaccagggctggtatgatatcaccaagaccttcaatg
gcaaagatgatttgctttgcggtgctgctaccgcaggcaacatgctgcattggtggtttgaccagaataaagagaagattgaaga
atacttgaagaagcacccggactacattaaaatcatgtttggtgatcaagagctgctggacgtgcgtaaagtgattaacaccaag
ggagaccaaaccaatagcgaactgttcaactacttccgcgataaagcgtttccgggtctgagcgcgcgtcgcattggcgtgatg
ccggacttggttctggatatgttcatcatgggttattacctaaatgtctataaaacccagaccaccgatgtgaaccgcacctaccag
gagaaggaccgtcgtggtggtatctttatcgcggtgttcacgttaggcgaccaaagcaaactgctcacgagccgtcacgacttca
aggagaagaacctgaaagagatcagcgacctgattaaaaaagagttgaccgaaggtaaggcgctgggcttgtcccataccta
cgcaaatgttcgtattaaccacgttatcaacctttggggtgcggacttcgattctaacggtaatctgaaggccatctacgtgacggat
tctgactccaacgccagcattggcatgaaaaagtattttgttggtgtcaactcggcgggtaaagtagcaattagcgctaaagagat
caaggaggacaacatcggcgcgcaggttctgggtctgttcacgctgtccaccggtcaggatctgtggaatatgaccctgtaa
IdeS^{mt7} protein sequence (340 aa + stop codon) (mutations underlined)
(SEQ ID NO: 30)
RKRCYSTSAVVLAAVTLFALSVDRGVIADSFSANQEIRYSEVTPYHVTSVWTKGVTPPAKFT
QGEDVFHAPYVANQGWYDITKTFNGKDDLLCGAATAGNMLHWWFDQNKEKIEEYLKKHP
DYIKIMFGDQELLDVRKVINTKGDQTNSELFNYFRDKAFPGLSARRIGVMPDLVLDMFIMGY
YLNVYKTQTTDVNRTYQEKDRRGGIFIAVFTLGDQSKLLTSRHDFKEKNLKEISDLIKKELTE
GKALGLSHTYANVRINHVINLWGADFDSNGNLKAIYVTDSDSNASIGMKKYFVGVNSAGKV
AISAKEIKEDNIGAQVLGLFTLSTGQDLWNMTL*
Plasmid pGEX-IdeS-mt8 (6,004 bp)
(SEQ ID NO: 31)
acgttatcgactgcacggtgcaccaatgcttctggcgtcaggcagccatcggaagctgtggtatggctgtgcaggtcgtaaatcac
tgcataattcgtgtcgctcaaggcgcactcccgttctggataatgttttttgcgccgacatcataacggttctggcaaatattctgaaat
gagctgttgacaattaatcatcggctcgtataatgtgtggaattgtgagcggataacaatttcacacaggaaacagtattcatgtcc
cctatactaggttattggaaaattaagggccttgtgcaacccactcgacttcttttggaatatcttgaagaaaaatatgaagagcattt
gtatgagcgcgatgaaggtgataaatggcgaaacaaaaagtttgaattgggtttggagtttcccaatcttccttattatattgatggtg
atgttaaattaacacagtctatggccatcatacgttatatagctgacaagcacaacatgttgggtggttgtccaaaagagcgtgca
gagatttcaatgcttgaaggagcggttttggatattagatacggtgtttcgagaattgcatatagtaaagactttgaaactctcaaagt
tgattttcttagcaagctacctgaaatgctgaaaatgttcgaagatcgtttatgtcataaaacatatttaaatggtgatcatgtaaccca
tcctgacttcatgttgtatgacgctcttgatgttgttttatacatggacccaatgtgcctggatgcgttcccaaaattagtttgttttaaaaa
acgtattgaagctatcccacaaattgataagtacttgaaatccagcaagtatatagcatggcctttgcagggctggcaagccacgt
ttggtggtggcgaccatcctccaaaatcggatctggaagttctgttccaggggcccctgggatccaggaaaagatgttattcaaca
agtgctgtagtgctggcagcggttactctgttcgccctgtctgttgatcgtggcgtgatcgccgattcttttagcgcgaatcaagaaatt
cgttacagcgaagttaccccgtaccatgtgacgtcggtgtggaccaagggtgtcacaccgccagcaaagttcacccaaggcga
agatgtgtttcatgcgccgtacgtggcgaaccagggttggtatgatattaccaagacctttaacggcaaagatgatttgctgtgtggt
gcggctaccgcaggaaacatgctgcattggtggttcgattgcaataaagagaagatcgaagcatatctgaagaagcacccgga
caaacagaaaatcatgtttggcgaccaagagttgctcgacgtacgtaaagttatcaacaccaaaggcgaccagacgaacagc
gaactgttcaactacttccgcgacaaagcgtttccgggtttgtcggcgcgtcgtatcggcgtgatgccggatttggttttggacatgtt
catcaacggttattacctgaatgtgtataaaacccagacgaccgatgttaatcgtacttaccaggagaaggaccgcagaggtggt
atttttgacgcagtctttacgcgcggtgaccaaagcaaactgttgacctctcgtcacgacttcaaagagaagaacctgaaagaga
tcagcgacctgattaaaaaggaacttaccgaaggtaaggcgctgggtttaagccacacctatgctaatgtacgcattaaccacgt
tatcaacctgtggggtgcggatttcgactccaatggcaacctgaaggccatctatgttacggattccgactccaacgccagcattg
gcatgaaaaaatactttgtcggtgtgaacagcgctggtaaggtcgcgatcagcgcgaaagagattaaggaggacaacattggc
gctcaggttctgggcctcttcaccctgagttgcggtcaggattcctggaatcaaaccaattaagaattcccgggtcgactcgagcg
gccgcatcgtgactgactgacgatctgcctcgcgcgtttcggtgatgacggtgaaaacctctgacacatgcagctcccggagac
ggtcacagcttgtctgtaagcggatgccgggagcagacaagcccgtcagggcgcgtcagcgggtgttggcgggtgtcggggc
gcagccatgacccagtcacgtagcgatagcggagtgtataattcttgaagacgaaagggcctcgtgatacgcctatttttataggtt
aatgtcatgataataatggtttcttagacgtcaggtggcacttttcggggaaatgtgcgcggaacccctatttgtttatttttctaaatac
attcaaatatgtatccgctcatgagacaataaccctgataaatgcttcaataatattgaaaaaggaagagtatgagtattcaacattt
ccgtgtcgcccttattcccttttttgcggcattttgccttcctgtttttgctcacccagaaacgctggtgaaagtaaaagatgctgaagat
cagttgggtgcacgagtgggttacatcgaactggatctcaacagcggtaagatccttgagagttttcgccccgaagaacgttttcc
aatgatgagcacttttaaagttctgctatgtggcgcggtattatcccgtgttgacgccgggcaagagcaactcggtcgccgcatac
actattctcagaatgacttggttgagtactcaccagtcacagaaaagcatcttacggatggcatgacagtaagagaattatgcagt
gctgccataaccatgagtgataacactgcggccaacttacttctgacaacgatcggaggaccgaaggagctaaccgcttttttgc
acaacatgggggatcatgtaactcgccttgatcgttgggaaccggagctgaatgaagccataccaaacgacgagcgtgacac
cacgatgcctgcagcaatggcaacaacgttgcgcaaactattaactggcgaactacttactctagcttcccggcaacaattaata
gactggatggaggcggataaagttgcaggaccacttctgcgctcggcccttccggctggctggtttattgctgataaatctggagc
cggtgagcgtgggtctcgcggtatcattgcagcactggggccagatggtaagccctcccgtatcgtagttatctacacgacgggg
agtcaggcaactatggatgaacgaaatagacagatcgctgagataggtgcctcactgattaagcattggtaactgtcagaccaa
gtttactcatatatactttagattgatttaaaacttcatttttaatttaaaaggatctaggtgaagatcctttttgataatctcatgaccaaa
atcccttaacgtgagttttcgttccactgagcgtcagaccccgtagaaaagatcaaaggatcttcttgagatcctttttttctgcgcgtaat
ctgctgcttgcaaacaaaaaaaccaccgctaccagcggtggtttgtttgccggatcaagagctaccaactctttttccgaaggtaa
ctggcttcagcagagcgcagataccaaatactgtccttctagtgtagccgtagttaggccaccacttcaagaactctgtagcaccg
cctacatacctcgctctgctaatcctgttaccagtggctgctgccagtggcgataagtcgtgtcttaccgggttggactcaagacgat
agttaccggataaggcgcagcggtcgggctgaacggggggttcgtgcacacagcccagcttggagcgaacgacctacaccg
aactgagatacctacagcgtgagctatgagaaagcgccacgcttcccgaagggagaaaggcggacaggtatccggtaagcg
gcagggtcggaacaggagagcgcacgagggagcttccagggggaaacgcctggtatctttatagtcctgtcgggtttcgccac
ctctgacttgagcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcctgatgcggtattttctcct
tacgcatctgtgcggtatttcacaccgcataaattccgacaccatcgaatggtgcaaaacctttcgcggtatggcatgatagcgcc
cggaagagagtcaattcagggtggtgaatgtgaaaccagtaacgttatacgatgtcgcagagtatgccggtgtctcttatcagac
cgtttcccgcgtggtgaaccaggccagccacgtttctgcgaaaacgcgggaaaaagtggaagcggcgatggcggagctgaat
tacattcccaaccgcgtggcacaacaactggcgggcaaacagtcgttgctgattggcgttgccacctccagtctggccctgcacg
cgccgtcgcaaattgtcgcggcgattaaatctcgcgccgatcaactgggtgccagcgtggtggtgtcgatggtagaacgaagcg
gcgtcgaagcctgtaaagcggcggtgcacaatcttctcgcgcaacgcgtcagtgggctgatcattaactatccgctggatgacca
ggatgccattgctgtggaagctgcctgcactaatgttccggcgttatttcttgatgtctctgaccagacacccatcaacagtattattttc
tcccatgaagacggtacgcgactgggcgtggagcatctggtcgcattgggtcaccagcaaatcgcgctgttagcgggcccatta
agttctgtctcggcgcgtctgcgtctggctggctggcataaatatctcactcgcaatcaaattcagccgatagcggaacgggaagg
cgactggagtgccatgtccggttttcaacaaaccatgcaaatgctgaatgagggcatcgttcccactgcgatgctggttgccaacg
atcagatggcgctgggcgcaatgcgcgccattaccgagtccgggctgcgcgttggtgcggatatctcggtagtgggatacgacg
ataccgaagacagctcatgttatatcccgccgtcaaccaccatcaaacaggattttcgcctgctggggcaaaccagcgtggacc
gcttgctgcaactctctcagggccaggcggtgaagggcaatcagctgttgcccgtctcactggtgaaaagaaaaaccaccctgg
cgcccaatacgcaaaccgcctctccccgcgcgttggccgattcattaatgcagctggcacgacaggtttcccgactggaaagcg
ggcagtgagcgcaacgcaattaatgtgagttagctcactcattaggcaccccaggctttacactttatgcttccggctcgtatgttgtg
tggaattgtgagcggataacaatttcacacaggaaacagctatgaccatgattacggattcactggccgtcgttttacaacgtcgtg
actgggaaaaccctggcgttacccaacttaatcgccttgcagcacatccccctttcgccagctggcgtaatagcgaagaggccc
gcaccgatcgcccttcccaacagttgcgcagcctgaatggcgaatggcgctttgcctggtttccggcaccagaagcggtgccgg
aaagctggctggagtgcgatcttcctgaggccgatactgtcgtcgtcccctcaaactggcagatgcacggttacgatgcgcccat
ctacaccaacgtaacctatcccattacggtcaatccgccgtttgttcccacggagaatccgacgggttgttactcgctcacatttaat
gttgatgaaagctggctacaggaaggccagacgcgaattatttttgatggcgttggaatt
IdeS^{mt8} DNA sequence (1,023 bp)
(SEQ ID NO: 32)
aggaaaagatgttattcaacaagtgctgtagtgctggcagcggttactctgttcgccctgtctgttgatcgtggcgtgatcgccgattc
ttttagcgcgaatcaagaaattcgttacagcgaagttaccccgtaccatgtgacgtcggtgtggaccaagggtgtcacaccgcca
gcaaagttcacccaaggcgaagatgtgtttcatgcgccgtacgtggcgaaccagggttggtatgatattaccaagacctttaacg
gcaaagatgatttgctgtgtggtgcggctaccgcaggaaacatgctgcattggtggttcgattgcaataaagagaagatcgaagc
atatctgaagaagcacccggacaaacagaaaatcatgtttggcgaccaagagttgctcgacgtacgtaaagttatcaacacca
aaggcgaccagacgaacagcgaactgttcaactacttccgcgacaaagcgtttccgggtttgtcggcgcgtcgtatcggcgtgat
gccggatttggttttggacatgttcatcaacggttattacctgaatgtgtataaaacccagacgaccgatgttaatcgtacttaccagg
agaaggaccgcagaggtggtatttttgacgcagtctttacgcgcggtgaccaaagcaaactgttgacctctcgtcacgacttcaa
agagaagaacctgaaagagatcagcgacctgattaaaaaggaacttaccgaaggtaaggcgctgggtttaagccacacctat
gctaatgtacgcattaaccacgttatcaacctgtggggtgcggatttcgactccaatggcaacctgaaggccatctatgttacggat
tccgactccaacgccagcattggcatgaaaaaatactttgtcggtgtgaacagcgctggtaaggtcgcgatcagcgcgaaaga
gattaaggaggacaacattggcgctcaggttctgggcctcttcaccctgagttgcggtcaggattcctggaatcaaaccaattaa
IdeS^{mt8} protein sequence (340 aa) (mutations underlined)
(SEQ ID NO: 33)
RKRCYSTSAVVLAAVTLFALSVDRGVIADSFSANQEIRYSEVTPYHVTSVWTKGVTPPAKFT
QGEDVFHAPYVANQGWYDITKTFNGKDDLLCGAATAGNMLHWWFDCNKEKIEAYLKKHP
DKQKIMFGDQELLDVRKVINTKGDQTNSELFNYFRDKAFPGLSARRIGVMPDLVLDMFINGY
YLNVYKTQTTDVNRTYQEKDRRGGIFDAVFTRGDQSKLLTSRHDFKEKNLKEISDLIKKELT
EGKALGLSHTYANVRINHVINLWGADFDSNGNLKAIYVTDSDSNASIGMKKYFVGVNSAGK
VAISAKEIKEDNIGAQVLGLFTLSCGQDSWNQTN
Plasmid pGEX-IdeS-mt9 (6,004 bp)
(SEQ ID NO: 34)
acgttatcgactgcacggtgcaccaatgcttctggcgtcaggcagccatcggaagctgtggtatggctgtgcaggtcgtaaatcac
tgcataattcgtgtcgctcaaggcgcactcccgttctggataatgttttttgcgccgacatcataacggttctggcaaatattctgaaat
gagctgttgacaattaatcatcggctcgtataatgtgtggaattgtgagcggataacaatttcacacaggaaacagtattcatgtcc
cctatactaggttattggaaaattaagggccttgtgcaacccactcgacttcttttggaatatcttgaagaaaaatatgaagagcattt
gtatgagcgcgatgaaggtgataaatggcgaaacaaaaagtttgaattgggtttggagtttcccaatcttccttattatattgatggtg
atgttaaattaacacagtctatggccatcatacgttatatagctgacaagcacaacatgttgggtggttgtccaaaagagcgtgca
gagatttcaatgcttgaaggagcggttttggatattagatacggtgtttcgagaattgcatatagtaaagactttgaaactctcaaagt
tgattttcttagcaagctacctgaaatgctgaaaatgttcgaagatcgtttatgtcataaaacatatttaaatggtgatcatgtaaccca
tcctgacttcatgttgtatgacgctcttgatgttgttttatacatggacccaatgtgcctggatgcgttcccaaaattagtttgttttaaaaa
acgtattgaagctatcccacaaattgataagtacttgaaatccagcaagtatatagcatggcctttgcagggctggcaagccacgt
ttggtggtggcgaccatcctccaaaatcggatctggaagttctgttccaggggcccctgggatccaggaaaagatgttattcaaca
agtgctgtagtcctggcggcagttactctgttcgccctgtccgtggaccgcggtgtgatcgcggactcctttagcgcgaatcaagag
atccgttatagcgaagttactccgtaccatgttacctctgtttggaccaagggtgttactccgccggcaaaattcacgcagggcgaa
gattgttttcacgctccgtatgtggcgaatcaaggctggtatgatattaccaaaacctttaacggaaaggatgatttgctttgcggtgc
ggctaccgcaggcaacatgctgcattggtggtttgaccagaataaagagaagattgaagcgtatctgaagaagcacccagaca
agcagaaaatcatgttcggtgaccaggagctgttagatgtgcgcaaagtcatcaacaccaaaggcgatcaaaccaactctgaa
ttgttcaactatttccgcgataaagcctttccgggtctctcggcgcgtcgtatcggcgtgatgccggacctggttctggacatgtttatc
aacggctactacctgaatgtttataaaacccaaaccaccgacgttaatcgtacctaccaggagaaggaccgtagaggcggtatt
ttcgacgccgtttttacgcgtggtgaccaaagcaaactgttgaccagccgtcacgacttcaaagaaaagaaccttaaggaaatc
agcgatctgattaaaaaagagctgaccgaaggtaaggctctgggcttgtcccatacgtacgcgaacgtacgcatcaaccacgt
gattaacctgtggggtgctgattttgacagcaacggtaatctgaaagcgatttacgttaccgatagcgattccaacgcgagcattgg
gatgaaaaagtacttcgtgggtgtcaacagcgcaggcaaggtggccatttcttgcaaagagatcaaggaggacaatatcggcg
cgcaggtgctcggtttgttcacgctgtcgaccggtcaggatagctggaatcaaacgaactaagaattcccgggtcgactcgagcg
gccgcatcgtgactgactgacgatctgcctcgcgcgtttcggtgatgacggtgaaaacctctgacacatgcagctcccggagac
ggtcacagcttgtctgtaagcggatgccgggagcagacaagcccgtcagggcgcgtcagcgggtgttggcgggtgtcggggc
gcagccatgacccagtcacgtagcgatagcggagtgtataattcttgaagacgaaagggcctcgtgatacgcctatttttataggtt
aatgtcatgataataatggtttcttagacgtcaggtggcacttttcggggaaatgtgcgcggaacccctatttgtttatttttctaaatac
attcaaatatgtatccgctcatgagacaataaccctgataaatgcttcaataatattgaaaaaggaagagtatgagtattcaacattt
ccgtgtcgcccttattcccttttttgcggcattttgccttcctgtttttgctcacccagaaacgctggtgaaagtaaaagatgctgaagat
cagttgggtgcacgagtgggttacatcgaactggatctcaacagcggtaagatccttgagagttttcgccccgaagaacgttttcc
aatgatgagcacttttaaagttctgctatgtggcgcggtattatcccgtgttgacgccgggcaagagcaactcggtcgccgcatac
actattctcagaatgacttggttgagtactcaccagtcacagaaaagcatcttacggatggcatgacagtaagagaattatgcagt
gctgccataaccatgagtgataacactgcggccaacttacttctgacaacgatcggaggaccgaaggagctaaccgcttttttgc
acaacatgggggatcatgtaactcgccttgatcgttgggaaccggagctgaatgaagccataccaaacgacgagcgtgacac
cacgatgcctgcagcaatggcaacaacgttgcgcaaactattaactggcgaactacttactctagcttcccggcaacaattaata
gactggatggaggcggataaagttgcaggaccacttctgcgctcggcccttccggctggctggtttattgctgataaatctggagc
cggtgagcgtgggtctcgcggtatcattgcagcactggggccagatggtaagccctcccgtatcgtagttatctacacgacgggg
agtcaggcaactatggatgaacgaaatagacagatcgctgagataggtgcctcactgattaagcattggtaactgtcagaccaa
gtttactcatatatactttagattgatttaaaacttcatttttaatttaaaaggatctaggtgaagatcctttttgataatctcatgaccaaa
atcccttaacgtgagttttcgttccactgagcgtcagaccccgtagaaaagatcaaaggatcttcttgagatcctttttttctgcgcgtaat
ctgctgcttgcaaacaaaaaaaccaccgctaccagcggtggtttgtttgccggatcaagagctaccaactctttttccgaaggtaa
ctggcttcagcagagcgcagataccaaatactgtccttctagtgtagccgtagttaggccaccacttcaagaactctgtagcaccg
cctacatacctcgctctgctaatcctgttaccagtggctgctgccagtggcgataagtcgtgtcttaccgggttggactcaagacgat
agttaccggataaggcgcagcggtcgggctgaacggggggttcgtgcacacagcccagcttggagcgaacgacctacaccg
aactgagatacctacagcgtgagctatgagaaagcgccacgcttcccgaagggagaaaggcggacaggtatccggtaagcg
gcagggtcggaacaggagagcgcacgagggagcttccagggggaaacgcctggtatctttatagtcctgtcgggtttcgccac
ctctgacttgagcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcctgatgcggtattttctcct
tacgcatctgtgcggtatttcacaccgcataaattccgacaccatcgaatggtgcaaaacctttcgcggtatggcatgatagcgcc
cggaagagagtcaattcagggtggtgaatgtgaaaccagtaacgttatacgatgtcgcagagtatgccggtgtctcttatcagac
cgtttcccgcgtggtgaaccaggccagccacgtttctgcgaaaacgcgggaaaaagtggaagcggcgatggcggagctgaat
tacattcccaaccgcgtggcacaacaactggcgggcaaacagtcgttgctgattggcgttgccacctccagtctggccctgcacg
cgccgtcgcaaattgtcgcggcgattaaatctcgcgccgatcaactgggtgccagcgtggtggtgtcgatggtagaacgaagcg
gcgtcgaagcctgtaaagcggcggtgcacaatcttctcgcgcaacgcgtcagtgggctgatcattaactatccgctggatgacca
ggatgccattgctgtggaagctgcctgcactaatgttccggcgttatttcttgatgtctctgaccagacacccatcaacagtattattttc
tcccatgaagacggtacgcgactgggcgtggagcatctggtcgcattgggtcaccagcaaatcgcgctgttagcgggcccatta
agttctgtctcggcgcgtctgcgtctggctggctggcataaatatctcactcgcaatcaaattcagccgatagcggaacgggaagg
cgactggagtgccatgtccggttttcaacaaaccatgcaaatgctgaatgagggcatcgttcccactgcgatgctggttgccaacg
atcagatggcgctgggcgcaatgcgcgccattaccgagtccgggctgcgcgttggtgcggatatctcggtagtgggatacgacg
ataccgaagacagctcatgttatatcccgccgtcaaccaccatcaaacaggattttcgcctgctggggcaaaccagcgtggacc
gcttgctgcaactctctcagggccaggcggtgaagggcaatcagctgttgcccgtctcactggtgaaaagaaaaaccaccctgg
cgcccaatacgcaaaccgcctctccccgcgcgttggccgattcattaatgcagctggcacgacaggtttcccgactggaaagcg
ggcagtgagcgcaacgcaattaatgtgagttagctcactcattaggcaccccaggctttacactttatgcttccggctcgtatgttgtg
tggaattgtgagcggataacaatttcacacaggaaacagctatgaccatgattacggattcactggccgtcgttttacaacgtcgtg
actgggaaaaccctggcgttacccaacttaatcgccttgcagcacatccccctttcgccagctggcgtaatagcgaagaggccc
gcaccgatcgcccttcccaacagttgcgcagcctgaatggcgaatggcgctttgcctggtttccggcaccagaagcggtgccgg
aaagctggctggagtgcgatcttcctgaggccgatactgtcgtcgtcccctcaaactggcagatgcacggttacgatgcgcccat
ctacaccaacgtaacctatcccattacggtcaatccgccgtttgttcccacggagaatccgacgggttgttactcgctcacatttaat
gttgatgaaagctggctacaggaaggccagacgcgaattatttttgatggcgttggaatt
IdeS^{mt9} DNA sequence (1,023 bp)
(SEQ ID NO: 35)
aggaaaagatgttattcaacaagtgctgtagtcctggcggcagttactctgttcgccctgtccgtggaccgcggtgtgatcgcgga
ctcctttagcgcgaatcaagagatccgttatagcgaagttactccgtaccatgttacctctgtttggaccaagggtgttactccgccg
gcaaaattcacgcagggcgaagattgttttcacgctccgtatgtggcgaatcaaggctggtatgatattaccaaaacctttaacgg
aaaggatgatttgctttgcggtgcggctaccgcaggcaacatgctgcattggtggtttgaccagaataaagagaagattgaagcg
tatctgaagaagcacccagacaagcagaaaatcatgttcggtgaccaggagctgttagatgtgcgcaaagtcatcaacaccaa
aggcgatcaaaccaactctgaattgttcaactatttccgcgataaagcctttccgggtctctcggcgcgtcgtatcggcgtgatgcc
ggacctggttctggacatgtttatcaacggctactacctgaatgtttataaaacccaaaccaccgacgttaatcgtacctaccagga
gaaggaccgtagaggcggtattttcgacgccgtttttacgcgtggtgaccaaagcaaactgttgaccagccgtcacgacttcaaa
gaaaagaaccttaaggaaatcagcgatctgattaaaaaagagctgaccgaaggtaaggctctgggcttgtcccatacgtacgc
gaacgtacgcatcaaccacgtgattaacctgtggggtgctgattttgacagcaacggtaatctgaaagcgatttacgttaccgata
gcgattccaacgcgagcattgggatgaaaaagtacttcgtgggtgtcaacagcgcaggcaaggtggccatttcttgcaaagaga
tcaaggaggacaatatcggcgcgcaggtgctcggtttgttcacgctgtcgaccggtcaggatagctggaatcaaacgaactaa
IdeS^{mt9} Protein sequence (340 aa) (mutations underlined)
(SEQ ID NO: 36)
RKRCYSTSAVVLAAVTLFALSVDRGVIADSFSANQEIRYSEVTPYHVTSVWTKGVTPPAKFT
QGEDCFHAPYVANQGWYDITKTFNGKDDLLCGAATAGNMLHWWFDQNKEKIEAYLKKHP
DKQKIMFGDQELLDVRKVINTKGDQTNSELFNYFRDKAFPGLSARRIGVMPDLVLDMFINGY
YLNVYKTQTTDVNRTYQEKDRRGGIFDAVFTRGDQSKLLTSRHDFKEKNLKEISDLIKKELT
EGKALGLSHTYANVRINHVINLWGADFDSNGNLKAIYVTDSDSNASIGMKKYFVGVNSAGK
VAISCKEIKEDNIGAQVLGLFTLSTGQDSWNQTN
Plasmid pGEX-IdeS-mt10 (6,004 bp)
(SEQ ID NO: 37)
acgttatcgactgcacggtgcaccaatgcttctggcgtcaggcagccatcggaagctgtggtatggctgtgcaggtcgtaaatcac
tgcataattcgtgtcgctcaaggcgcactcccgttctggataatgttttttgcgccgacatcataacggttctggcaaatattctgaaat
gagctgttgacaattaatcatcggctcgtataatgtgtggaattgtgagcggataacaatttcacacaggaaacagtattcatgtcc
cctatactaggttattggaaaattaagggccttgtgcaacccactcgacttcttttggaatatcttgaagaaaaatatgaagagcattt
gtatgagcgcgatgaaggtgataaatggcgaaacaaaaagtttgaattgggtttggagtttcccaatcttccttattatattgatggtg
atgttaaattaacacagtctatggccatcatacgttatatagctgacaagcacaacatgttgggtggttgtccaaaagagcgtgca
gagatttcaatgcttgaaggagcggttttggatattagatacggtgtttcgagaattgcatatagtaaagactttgaaactctcaaagt
tgattttcttagcaagctacctgaaatgctgaaaatgttcgaagatcgtttatgtcataaaacatatttaaatggtgatcatgtaaccca
tcctgacttcatgttgtatgacgctcttgatgttgttttatacatggacccaatgtgcctggatgcgttcccaaaattagtttgttttaaaaa
acgtattgaagctatcccacaaattgataagtacttgaaatccagcaagtatatagcatggcctttgcagggctggcaagccacgt
ttggtggtggcgaccatcctccaaaatcggatctggaagttctgttccaggggcccctgggatccaggaaaagatgttattcaaca
agtgctgtagtacttgcggctgtgaccctgttcgccttgagtgttgatcgcggcgtcatcgccgatagctttagcgcgaaccaagaa
atccgttatagcgaagttaccccgtaccacgttacgtcttgttggaccaagggcgtgacgccaccggcaaaattcacgcagggtg
aggacgtttttcacgcaccgtacgtggcgaaccaaggttggtatgacatcaccaagacctttaacggcaaagacgatctgctttgc
ggtgcagcgaccgcaggcaacatgctgcattggtggtttgaccagaataaagaaaagatcgaagcatacctgaagaagcacc
cggataaacagaaaatcatgttcggcgaccaagagttattggacgttcgtaaggtgatcaacaccaaaggcgaccagactaat
agcgaactgttcaattattttcgcgacaaagcgtttccgggtttgtccgctcgtcgcattggcgtcatgccggatctggttctggacatg
ttcatcaacggttattacttgaatgtttataaaactcaaacgaccgacgtcaatagaacgtaccaggagaaggaccgtcgtggcg
gcatttttgacgctgtgtttacccgtggcgatcaaagcaaactgttgaccagccgtcatgatttcaaagagaagaacctcaaggag
atctccgatctgattaaaaaggagtgcaccgaaggtaaagcgctgggtctgagccatacctacgctaacgtgcgcatcaaccac
gtgattaatctgtggggtgccgatttcgattccaacggtaacctgaaggcgatttatgttaccgactcggactctaatgcgagcattg
gcatgaaaaagtacttcgtgggtgtgaacagcgcgggtaaggtggctattagcgcgaaagaaatcaaggaggacaacattgg
tgcgcaggttctgggtttgttcactctgtccaccggtcaggattcttggaatcaaaccaactaagaattcccgggtcgactcgagcg
gccgcatcgtgactgactgacgatctgcctcgcgcgtttcggtgatgacggtgaaaacctctgacacatgcagctcccggagac
ggtcacagcttgtctgtaagcggatgccgggagcagacaagcccgtcagggcgcgtcagcgggtgttggcgggtgtcggggc
gcagccatgacccagtcacgtagcgatagcggagtgtataattcttgaagacgaaagggcctcgtgatacgcctatttttataggtt
aatgtcatgataataatggtttcttagacgtcaggtggcacttttcggggaaatgtgcgcggaacccctatttgtttatttttctaaatac
attcaaatatgtatccgctcatgagacaataaccctgataaatgcttcaataatattgaaaaaggaagagtatgagtattcaacattt
ccgtgtcgcccttattcccttttttgcggcattttgccttcctgtttttgctcacccagaaacgctggtgaaagtaaaagatgctgaagat
cagttgggtgcacgagtgggttacatcgaactggatctcaacagcggtaagatccttgagagttttcgccccgaagaacgttttcc
aatgatgagcacttttaaagttctgctatgtggcgcggtattatcccgtgttgacgccgggcaagagcaactcggtcgccgcatac
actattctcagaatgacttggttgagtactcaccagtcacagaaaagcatcttacggatggcatgacagtaagagaattatgcagt
gctgccataaccatgagtgataacactgcggccaacttacttctgacaacgatcggaggaccgaaggagctaaccgcttttttgc
acaacatgggggatcatgtaactcgccttgatcgttgggaaccggagctgaatgaagccataccaaacgacgagcgtgacac
cacgatgcctgcagcaatggcaacaacgttgcgcaaactattaactggcgaactacttactctagcttcccggcaacaattaata
gactggatggaggcggataaagttgcaggaccacttctgcgctcggcccttccggctggctggtttattgctgataaatctggagc
cggtgagcgtgggtctcgcggtatcattgcagcactggggccagatggtaagccctcccgtatcgtagttatctacacgacgggg
agtcaggcaactatggatgaacgaaatagacagatcgctgagataggtgcctcactgattaagcattggtaactgtcagaccaa
gtttactcatatatactttagattgatttaaaacttcatttttaatttaaaaggatctaggtgaagatcctttttgataatctcatgaccaaa
atcccttaacgtgagttttcgttccactgagcgtcagaccccgtagaaaagatcaaaggatcttcttgagatcctttttttctgcgcgtaat
ctgctgcttgcaaacaaaaaaaccaccgctaccagcggtggtttgtttgccggatcaagagctaccaactctttttccgaaggtaa
ctggcttcagcagagcgcagataccaaatactgtccttctagtgtagccgtagttaggccaccacttcaagaactctgtagcaccg
cctacatacctcgctctgctaatcctgttaccagtggctgctgccagtggcgataagtcgtgtcttaccgggttggactcaagacgat
agttaccggataaggcgcagcggtcgggctgaacggggggttcgtgcacacagcccagcttggagcgaacgacctacaccg
aactgagatacctacagcgtgagctatgagaaagcgccacgcttcccgaagggagaaaggcggacaggtatccggtaagcg
gcagggtcggaacaggagagcgcacgagggagcttccagggggaaacgcctggtatctttatagtcctgtcgggtttcgccac
ctctgacttgagcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcctgatgcggtattttctcct
tacgcatctgtgcggtatttcacaccgcataaattccgacaccatcgaatggtgcaaaacctttcgcggtatggcatgatagcgcc
cggaagagagtcaattcagggtggtgaatgtgaaaccagtaacgttatacgatgtcgcagagtatgccggtgtctcttatcagac
cgtttcccgcgtggtgaaccaggccagccacgtttctgcgaaaacgcgggaaaaagtggaagcggcgatggcggagctgaat
tacattcccaaccgcgtggcacaacaactgggggcaaacagtcgttgctgattggcgttgccacctccagtctggccctgcacg
cgccgtcgcaaattgtcgcggcgattaaatctcgcgccgatcaactgggtgccagcgtggtggtgtcgatggtagaacgaagcg
gcgtcgaagcctgtaaagcggcggtgcacaatcttctcgcgcaacgcgtcagtgggctgatcattaactatccgctggatgacca
ggatgccattgctgtggaagctgcctgcactaatgttccggcgttatttcttgatgtctctgaccagacacccatcaacagtattattttc
tcccatgaagacggtacgcgactgggcgtggagcatctggtcgcattgggtcaccagcaaatcgcgctgttagcgggcccatta
agttctgtctcggcgcgtctgcgtctggctggctggcataaatatctcactcgcaatcaaattcagccgatagcggaacgggaagg
cgactggagtgccatgtccggttttcaacaaaccatgcaaatgctgaatgagggcatcgttcccactgcgatgctggttgccaacg
atcagatggcgctgggcgcaatgcgcgccattaccgagtccgggctgcgcgttggtgcggatatctcggtagtgggatacgacg
ataccgaagacagctcatgttatatcccgccgtcaaccaccatcaaacaggattttcgcctgctggggcaaaccagcgtggacc
gcttgctgcaactctctcagggccaggcggtgaagggcaatcagctgttgcccgtctcactggtgaaaagaaaaaccaccctgg
cgcccaatacgcaaaccgcctctccccgcgcgttggccgattcattaatgcagctggcacgacaggtttcccgactggaaagcg
ggcagtgagcgcaacgcaattaatgtgagttagctcactcattaggcaccccaggctttacactttatgcttccggctcgtatgttgtg
tggaattgtgagcggataacaatttcacacaggaaacagctatgaccatgattacggattcactggccgtcgttttacaacgtcgtg
actgggaaaaccctggcgttacccaacttaatcgccttgcagcacatccccctttcgccagctggcgtaatagcgaagaggccc
gcaccgatcgcccttcccaacagttgcgcagcctgaatggcgaatggcgctttgcctggtttccggcaccagaagcggtgccgg
aaagctggctggagtgcgatcttcctgaggccgatactgtcgtcgtcccctcaaactggcagatgcacggttacgatgcgcccat
ctacaccaacgtaacctatcccattacggtcaatccgccgtttgttcccacggagaatccgacgggttgttactcgctcacatttaat
gttgatgaaagctggctacaggaaggccagacgcgaattatttttgatggcgttggaatt
Ides^{mt10} DNA sequence (1,023 bp)
(SEQ ID NO: 38)
aggaaaagatgttattcaacaagtgctgtagtacttgcggctgtgaccctgttcgccttgagtgttgatcgcggcgtcatcgccgata
gctttagcgcgaaccaagaaatccgttatagcgaagttaccccgtaccacgttacgtcttgttggaccaagggcgtgacgccacc
ggcaaaattcacgcagggtgaggacgtttttcacgcaccgtacgtggcgaaccaaggttggtatgacatcaccaagacctttaa
cggcaaagacgatctgctttgcggtgcagcgaccgcaggcaacatgctgcattggtggtttgaccagaataaagaaaagatcg
aagcatacctgaagaagcacccggataaacagaaaatcatgttcggcgaccaagagttattggacgttcgtaaggtgatcaac
accaaaggcgaccagactaatagcgaactgttcaattattttcgcgacaaagcgtttccgggtttgtccgctcgtcgcattggcgtc
atgccggatctggttctggacatgttcatcaacggttattacttgaatgtttataaaactcaaacgaccgacgtcaatagaacgtacc
aggagaaggaccgtcgtggcggcatttttgacgctgtgtttacccgtggcgatcaaagcaaactgttgaccagccgtcatgatttc
aaagagaagaacctcaaggagatctccgatctgattaaaaaggagtgcaccgaaggtaaagcgctgggtctgagccatacct
acgctaacgtgcgcatcaaccacgtgattaatctgtggggtgccgatttcgattccaacggtaacctgaaggcgatttatgttaccg
actcggactctaatgcgagcattggcatgaaaaagtacttcgtgggtgtgaacagcgcgggtaaggtggctattagcgcgaaag
aaatcaaggaggacaacattggtgcgcaggttctgggtttgttcactctgtccaccggtcaggattcttggaatcaaaccaactaa
Ides^{mt10} Protein sequence (340 aa + stop codon) (mutations underlined)
(SEQ ID NO: 39)
RKRCYSTSAVVLAAVTLFALSVDRGVIADSFSANQEIRYSEVTPYHVTSCWTKGVTPPAKFT
QGEDVFHAPYVANQGWYDITKTFNGKDDLLCGAATAGNMLHWWFDQNKEKIEAYLKKHP
DKQKIMFGDQELLDVRKVINTKGDQTNSELFNYFRDKAFPGLSARRIGVMPDLVLDMFINGY
YLNVYKTQTTDVNRTYQEKDRRGGIFDAVFTRGDQSKLLTSRHDFKEKNLKEISDLIKKECT
EGKALGLSHTYANVRINHVINLWGADFDSNGNLKAIYVTDSDSNASIGMKKYFVGVNSAGK
VAISAKEIKEDNIGAQVLGLFTLSTGQDSWNQTN*

1. A recombinant nucleic acid comprising a sequence encoding Streptococcus pyogenes IgG degrading enzyme (IdeS) that is codon-optimized for expression in E. coli cells, wherein the recombinant nucleic acid comprises a nucleotide sequence at least 90% identical to SEQ ID NO:1 or SEQ ID NO:2.

2. The recombinant nucleic acid of claim 1, comprising the nucleotide sequence of SEQ ID NO:1 or SEQ ID NO:2.

3. A recombinant nucleic acid comprising a nucleotide sequence encoding a modified Streptococcus pyogenes IgG degrading enzyme (IdeS), wherein the modified IdeS comprises a cysteine substitution at two residues to enable disulfide bond formation.

4. The recombinant nucleic acid of claim 3, comprising a nucleotide sequence at least 90% identical to SEQ ID NO:32, SEQ ID NO:35, or SEQ ID NO:38.

5. A recombinant nucleic acid comprising a nucleotide sequence encoding a modified Streptococcus pyogenes IgG degrading enzyme (IdeS), wherein the nucleotide sequence is at least 90% identical to SEQ ID NO:23 or SEQ ID NO:26.

6. The recombinant nucleic acid of claim 3, wherein the sequence encoding Streptococcus pyogenes IgG degrading enzyme (IdeS) is codon-optimized for expression in E. coli cells.

7. The recombinant nucleic acid of claim 1, wherein the recombinant nucleic acid is operably linked to a constitutive promoter.

8. The recombinant nucleic acid of claim 1, wherein the recombinant nucleic acid is operably linked to an inducible promoter.

9. A vector comprising the recombinant nucleic acid of claim 1.

10. The vector of claim 9, which is a plasmid.

11. The vector of claim 9, comprising the sequence of SEQ ID NO:3 or SEQ ID NO:4 or a sequence at least 90% identical thereto.

12. A cell in vitro comprising the recombinant nucleic acid of claim 1.

13. (canceled)

14. A recombinant IdeS produced from the recombinant nucleic acid of claim 1.

15. (canceled)

16. A pharmaceutical formulation comprising the recombinant IdeS of claim 14 and a pharmaceutically acceptable carrier.

17. A method of producing recombinant or modified IdeS, the method comprising expressing the IdeS from the recombinant nucleic acid of claim 1.

18-19. (canceled)

20. A method of depleting antibodies in a subject, comprising administering to the subject an effective amount of the recombinant IdeS of claim 14, thereby depleting antibodies in the subject.

21. A method of inhibiting binding of a heterologous agent by antibodies upon administration of the heterologous agent to a subject, comprising administering to the subject an effective amount of the recombinant IdeS of claim 14, thereby inhibiting binding of the heterologous agent by antibodies.

22-35. (canceled)

36. A method of expressing a polypeptide or functional nucleic acid in a subject, comprising administering to the subject (a) a nucleic acid delivery vector encoding the polypeptide or functional nucleic acid, and (b) an effective amount of the recombinant IdeS of claim 14, thereby expressing the polypeptide or functional nucleic acid in the subject.

37. A method of editing a gene in a subject, comprising administering to the subject (a) a gene editing complex, and (b) an effective amount of the recombinant IdeS of claim 14, thereby expressing the polypeptide or functional nucleic acid in the subject.

38. A method of treating an autoimmune disease in a subject in need thereof, comprising administering to the subject an effective amount of the recombinant IdeS of claim 14, thereby treating the autoimmune disease.

39-43. (canceled)
